SEQUENCE LISTING (198 sequences)

SEQ ID NO: 1 - DNA, 945 bp, Homo sapiens
acatttcttagtatagcatatactaagtatggccagactctgggttttgactctcgctgcttgtctccattcaggcaacagcctggctcccaccttggctgcttggttaactttgcatctttcaaacccatctctggactcagagcatcccgaaagtttaccctcacctctccggatggggcggtcattccccagtctggtttcaacatcaccgtccatcatacaatcgccatcacagcattcaccaactcgtgcagtgaggctgttctcgtaatttcccagctattctgggacctccttcctctaatggaccagagctgtggagtttgtggaacgaacgaatgagggctgacgtcacggtagcagatcgcaaccaccactctgggtccagggtcagaaggggtagggaaaagggatctgggcagctctagcaggcaccactcacaggtcgagagcgtgaagcaccacaagagacctggcccaggatgcgtagggccatgggtccgcaccccgcccactctgcccacccccagctcaatcttggcactgcctgcgaggaccctgccttgctccacaccaccctaccctttgtgaaccctctcggcggccgccatcttgcttccagcgcttccgccggaagcccgcaccgtgccccaccccatccggcaccagattaaggaaagaaggggccgtccggaaacaaggacatctcctttagatctgttaaggtgacaccagatttttttttttttttttttgagacggagtctcgctctgtcgcccaggctggagtgcagtggcgcgatctcggctcactgctagctccgcctcccaggttcacgccattctcctgcctcagcctcccaagaagttgtaactacaggtgcccgccaccacgcccggctaattttttttgtatttttagtagagacggggtttcaccgt

SEQ ID NO: 2 - DNA, 945 bp, Artificial Sequence
  Bisulfite converted genomic DNA from chr140024721-40025665
atattttttagtatagtatatattaagtatggttagattttgggttttgattttcgttgtttgtttttatttaggtaatagtttggtttttattttggttgtttggttaattttgtattttttaaatttatttttggatttagagtatttcgaaagtttatttttattttttcggatggggcggttattttttagtttggttttaatattatcgtttattatataatcgttattatagtatttattaattcgtgtagtgaggttgttttcgtaattttttagttattttgggattttttttttttaatggattagagttgtggagtttgtggaacgaacgaatgagggttgacgttacggtagtagatcgtaattattattttgggtttagggttagaaggggtagggaaaagggatttgggtagttttagtaggtattatttataggtcgagagcgtgaagtattataagagatttggtttaggatgcgtagggttatgggttcgtatttcgtttattttgtttatttttagtttaattttggtattgtttgcgaggattttgttttgttttatattattttattttttgtgaattttttcggcggtcgttattttgtttttagcgttttcgtcggaagttcgtatcgtgttttattttattcggtattagattaaggaaagaaggggtcgttcggaaataaggatattttttttagatttgttaaggtgatattagatttttttttttttttttttgagacggagtttcgttttgtcgtttaggttggagtgtagtggcgcgatttcggtttattgttagtttcgttttttaggtttacgttatttttttgttttagttttttaagaagttgtaattataggtgttcgttattacgttcggttaattttttttgtatttttagtagagacggggttttatcgt

SEQ ID NO: 3 - DNA, 22 bp, Artificial Sequence
  F1 Primer for Bisulfite converted genomic DNA from chr140024721-40025665
ggatggggcggttattttttag

SEQ ID NO: 4 - DNA, 21 bp, Artificial Sequence
  R1 Primer for Bisulfite converted genomic DNA from chr140024721-40025665
gaaatcgcgccactacactcc

SEQ ID NO: 5 - DNA, 517 bp, Homo sapiens
cgctctgtcgcccaggctggagtgcagtggcgcgatctctgctcactgcaacctttgcctcccgggttcaagcaattctcctgcctcaggctcccgagtagctggaattacaggcgcccgccgccacgcccggctaattttttaattttttaacagagacgggctttcaccatgttggccaggttggtctcgaactcccaacctcaagcgatccacccgcctcggcctcccaaagtgctgggattacagacgcgaaccaccgcgcccgactgtcctgtgttttaatgcttgaaaataatctgaatatggaaaatgagtgcacatatcccttgatgttttataaataacctacagtcctaataatggcttggcttagagttccttcacaacaggaaaagaaggcatcctggctttttttttttttttttttttaaatcaaagtgaggcttgggttggctctatcttggtcctgagtgcgaggaaggagggccgcaatgcatgctggatcttgcagttc

SEQ ID NO: 6 - DNA, 517 bp, Artificial Sequence
  Bisulfite converted genomic DNA from chr163248947-63249463
cgttttgtcgtttaggttggagtgtagtggcgcgatttttgtttattgtaatttttgtttttcgggtttaagtaatttttttgttttaggttttcgagtagttggaattataggcgttcgtcgttacgttcggttaattttttaattttttaatagagacgggtttttattatgttggttaggttggtttcgaatttttaattttaagcgatttattcgtttcggttttttaaagtgttgggattatagacgcgaattatcgcgttcgattgttttgtgttttaatgtttgaaaataatttgaatatggaaaatgagtgtatatattttttgatgttttataaataatttatagttttaataatggtttggtttagagtttttttataataggaaaagaaggtattttggttttttttttttttttttttttaaattaaagtgaggtttgggttggttttattttggttttgagtgcgaggaaggagggtcgtaatgtatgttggattttgtagttt

SEQ ID NO: 7 - DNA, 20 bp, Artificial Sequence
  F1 Primer for Bisulfite converted genomic DNA from chr163248947-63249463
taggcgttcgtcgttacgtt

SEQ ID NO: 8 - DNA, 21 bp, Artificial Sequence
  R1 Primer for Bisulfite converted genomic DNA from chr163248947-63249463
tccttcctcgcactcaaaacc

SEQ ID NO: 9 - DNA, 771 bp, Homo sapiens
aactgttatttgaacagcaaaaagaccaggcaaacatccgagagacagacctaactgcagataagtcttgtaatcttgttcactatagtcaaaacagaatggattgcttattctgttaccctattggtattgctcacattttaaaatttaggtgctgggctcaagaatgataattttattattaagcttcaagagtcacattgtattgtatgcattgaatatcacgaaatcgaaaacactgaaaagctagcgtttacattcttggatcatacccccaaaggccaaggattcccactggccgcttgcagcagggctgtctcctgtgaaggcaagagacaaacgaggagcgttatgacactttgctcccaggactttggcgcacagttcccatgagtccaggtccgtccagtcaacatcctgaagccaggactggcaggtcctgaccctccctttttgcacccgtaaggggaagggcccgggaatctggaccttgtctagagtcccggcgccagtaccagttcggagggcatcctggaggttgttactaacccgagcctcttccaggcttcccaggcaccagggttgctaacagctccacgggggcgaataattttgtttattttgttaactgtggttatctccaacactcagttcagacctgagccgggcaaagtctcaccttgtctcctggtcacttccctgcttgtttgtttcttcctaactcccaagtgtctttcgcccagttcatccccacctcagcagcccaaggct

SEQ ID NO: 10 - DNA, 771 bp, Artificial Sequence
  Bisulfite converted genomic DNA from chr195698577-95699347
aattgttatttgaatagtaaaaagattaggtaaatattcgagagatagatttaattgtagataagttttgtaattttgtttattatagttaaaatagaatggattgtttattttgttattttattggtattgtttatattttaaaatttaggtgttgggtttaagaatgataattttattattaagttttaagagttatattgtattgtatgtattgaatattacgaaatcgaaaatattgaaaagttagcgtttatatttttggattatatttttaaaggttaaggatttttattggtcgtttgtagtagggttgttttttgtgaaggtaagagataaacgaggagcgttatgatattttgtttttaggattttggcgtatagtttttatgagtttaggttcgtttagttaatattttgaagttaggattggtaggttttgattttttttttttgtattcgtaaggggaagggttcgggaatttggattttgtttagagtttcggcgttagtattagttcggagggtattttggaggttgttattaattcgagttttttttaggttttttaggtattagggttgttaatagttttacgggggcgaataattttgtttattttgttaattgtggttatttttaatatttagtttagatttgagtcgggtaaagttttattttgttttttggttatttttttgtttgtttgtttttttttaatttttaagtgtttttcgtttagtttatttttattttagtagtttaaggtt

SEQ ID NO: 11 - DNA, 24 bp, Artificial Sequence
  F1 Primer for Bisulfite converted genomic DNA from chr195698577-95699347
acgaaatcgaaaatattgaaaagt

SEQ ID NO: 12 - DNA, 21 bp, Artificial Sequence
  R1 Primer for Bisulfite converted genomic DNA from chr195698577-95699347
attattcgcccccgtaaaact

SEQ ID NO: 13 - DNA, 1482 bp, Homo sapiens
cacgcggccccacacggtattcctgtgggatcttacagagggaacccccctcacccttactggatggtagtgagggtgcatatccttgtcacccaccacccctcaccaagtgtagcagccccgctgggcctccagcaggaaaggcacccggcctggctctcggccgaagggtagcaacacctgtggcacgttaagtttgttggccagggtcccacgcaaaaccagaaacaacaacaggaggcggtgtagacgcaagaagaaaaagagcccgaagcctcggcgtcttgatgacgccggacagccagtcatggcgactgccaggtctcgggttcccgctcaactacagccggctcacagctccatcagccaatccacaggcgtgacgtcagcagagggggatgggacctgcttcttgctcggatgtgtgggcatttttaaatgcagtccttcagcaagccaatataatggaaacttttggcacaaacccagctaaaacacctcaaactctcttaaagggctttcctaaatactggatggcagacctttttaacagtaaggaacaagcagaaaagtctaaaaaaaaccctttaatttcttccagttttaatttcttctgttgcacactgtttatcttaacaccacaccctccgcccgacaaaaaaactttttgcaatgatttccagttagcattggtccatgcctatcaacggaaaattgtgcagtctctgggataaacagctcgactttgtcaagggaaccgtgtatttttactagaagtcagattcacgtccggcctccaggccagaatgcaggaaatgccagacactggcgtctcaaaagcagctttcaaagttatccactcacacagccggcctctaagcaaatactcttgagagcacttgttgttttctccaaatatccaaacagaatataaactcaaagctgtttcagtacagttggttaaaaccacgcatttatagtagtgatcagcattatatattgttttaaggagcataaatccatagtttataaggattgatatggttgtgaaggaaaatgtatatagttcagcctgagatgtcagtgttaacctataaaaggcccagcacattaagtgttctttctaatcttcatgtttactctgacaatacttatctccattttacagatgataaaactaaacatttgctctatattaaatcctgttgctccagctgggtgcggtggctcacgcccgtaatcccagcattttgggaagctgaggcaggtggatcacctgaaggtcaggagttcgagaccagcctggccaacatggtgaagccctgtctctacttaaaatataaaaaattacccgggcatggtggcaggtgcctgtaatcccagctactcgggaggctgaggcaggagaatctcttgaaccaggaggcggaggttgcagtgagccgagatcgagccattacgctccagcccaggcaacaagaa

SEQ ID NO: 14 - DNA, 1482 bp, Artificial Sequence
  Bisulfite converted genomic DNA from chr1154127212-154128693
tacgcggttttatacggtatttttgtgggattttatagagggaattttttttatttttattggatggtagtgagggtgtatatttttgttatttattattttttattaagtgtagtagtttcgttgggtttttagtaggaaaggtattcggtttggttttcggtcgaagggtagtaatatttgtggtacgttaagtttgttggttagggttttacgtaaaattagaaataataataggaggcggtgtagacgtaagaagaaaaagagttcgaagtttcggcgttttgatgacgtcggatagttagttatggcgattgttaggtttcgggttttcgtttaattatagtcggtttatagttttattagttaatttataggcgtgacgttagtagagggggatgggatttgttttttgttcggatgtgtgggtatttttaaatgtagttttttagtaagttaatataatggaaatttttggtataaatttagttaaaatattttaaatttttttaaagggtttttttaaatattggatggtagatttttttaatagtaaggaataagtagaaaagtttaaaaaaaattttttaattttttttagttttaattttttttgttgtatattgtttattttaatattatatttttcgttcgataaaaaaattttttgtaatgatttttagttagtattggtttatgtttattaacggaaaattgtgtagtttttgggataaatagttcgattttgttaagggaatcgtgtatttttattagaagttagatttacgttcggtttttaggttagaatgtaggaaatgttagatattggcgttttaaaagtagtttttaaagttatttatttatatagtcggtttttaagtaaatatttttgagagtatttgttgttttttttaaatatttaaatagaatataaatttaaagttgttttagtatagttggttaaaattacgtatttatagtagtgattagtattatatattgttttaaggagtataaatttatagtttataaggattgatatggttgtgaaggaaaatgtatatagtttagtttgagatgttagtgttaatttataaaaggtttagtatattaagtgttttttttaatttttatgtttattttgataatatttatttttattttatagatgataaaattaaatatttgttttatattaaattttgttgttttagttgggtgcggtggtttacgttcgtaattttagtattttgggaagttgaggtaggtggattatttgaaggttaggagttcgagattagtttggttaatatggtgaagttttgtttttatttaaaatataaaaaattattcgggtatggtggtaggtgtttgtaattttagttattcgggaggttgaggtaggagaattttttgaattaggaggcggaggttgtagtgagtcgagatcgagttattacgttttagtttaggtaataagaa

SEQ ID NO: 15 - DNA, 20 bp, Artificial Sequence
  F1 Primer for Bisulfite converted genomic DNA from chr1154127212-154128693
aataggaggcggtgtagacg

SEQ ID NO: 16 - DNA, 23 bp, Artificial Sequence
  R1 Primer for Bisulfite converted genomic DNA from chr1154127212-154128693
tcccaaaaactacacaattttcc

SEQ ID NO: 17 - DNA, 22 bp, Artificial Sequence
  F2 Primer for Bisulfite converted genomic DNA from chr1154127212-154128693
ggaaaattgtgtagtttttggg

SEQ ID NO: 18 - DNA, 23 bp, Artificial Sequence
  R2 Primer for Bisulfite converted genomic DNA from chr1154127212-154128693
aaccaaactaatctcgaactcct

SEQ ID NO: 19 - DNA, 784 bp, Homo sapiens
aaggtgggaaaactcgaagcgggaattggtggggggagaaatgggtccttccaggttataggtagatttaaaatttttctaattggcaattggttgaaagagttatcaatagaaaggaatgtctgggttgtcataagaacttgtggagaccaaagttttgtcatgcagatgaagcctccaggtaccaggcttccaagaaaatagattgtaaatgtttcttaccagaccgaaggtctgtgttgaccttaaccggagaggtatgaggctagtcggacccccacttcccgtcatggactgaaccagaattttagagtgccctggcctgggagaaagtccattccgatggttggcgacacggggggcttagaattgtattttcggtttacatctaaaagaagaacaaaggtctaggcttccagaatcatgggttctggtgaccgtggacctggaggtgatggtaaactctctttttgttcctggggtgttttccttgggatcctttctatgagaaatgtaggccgggttttatttttcgggttcctagtggtgaggtaaggtgaggtgcctcaacaagaaaaaggaccagttttggcttcaggtctatgtgaaagcggttaacactgggcctggaatggtagatactcaataattaaggttacttcttattctcggttcttttttccaaacctagcgcgtctaatctcttctaggccccgccccttctgagccccccctccttcggcctgtatgataggctcttcctccatttccggcttctgggact

SEQ ID NO: 20 - DNA, 784 bp, Artificial Sequence
  Bisulfite converted genomic DNA from chr1162466830-162467613
aaggtgggaaaattcgaagcgggaattggtggggggagaaatgggtttttttaggttataggtagatttaaaatttttttaattggtaattggttgaaagagttattaatagaaaggaatgtttgggttgttataagaatttgtggagattaaagttttgttatgtagatgaagtttttaggtattaggtttttaagaaaatagattgtaaatgttttttattagatcgaaggtttgtgttgattttaatcggagaggtatgaggttagtcggatttttatttttcgttatggattgaattagaattttagagtgttttggtttgggagaaagtttatttcgatggttggcgatacggggggtttagaattgtattttcggtttatatttaaaagaagaataaaggtttaggtttttagaattatgggttttggtgatcgtggatttggaggtgatggtaaattttttttttgtttttggggtgttttttttgggattttttttatgagaaatgtaggtcgggttttatttttcgggtttttagtggtgaggtaaggtgaggtgttttaataagaaaaaggattagttttggttttaggtttatgtgaaagcggttaatattgggtttggaatggtagatatttaataattaaggttattttttattttcggtttttttttttaaatttagcgcgtttaattttttttaggtttcgttttttttgagttttttttttttcggtttgtatgataggttttttttttattttcggtttttgggatt

SEQ ID NO: 21 - DNA, 23 bp, Artificial Sequence
  F1 Primer for Bisulfite converted genomic DNA from chr1162466830-162467613
gatcgaaggtttgtgttgatttt

SEQ ID NO: 22 - DNA, 21 bp, Artificial Sequence
  R1 Primer for Bisulfite converted genomic DNA from chr1162466830-162467613
cacctcaccttacctcaccac

SEQ ID NO: 23 - DNA, 1249 bp, Homo sapiens
atttagtgtgtgttctcagcctcaccattgacttgctgtgttaccttggacaagtcactctcccactctgacctcaattttcacatctctaaaaggagactgctagactagacggtgagtgaagtcctgctgagccctccgagttcattctcccttgtccagtcagtccttccctcccaccctcccctctgggctccctctctggggccctctcccccaggggaggcctgctccagccccaggggagctgcggccctggctcctgttcccagtcggagtgaccagccctgggcctccttcccagcttcggccctggctccatgtggcccatctgagaggctggcctcaagcccggcggcaactccacatttctgtttttccttttttttccctctttcccggagttaacaagaagcagatgtggcgcacgatggttggagaggtggggggaggaagggggaggccggaccgccagccagacaggggggaagggaggggagccagcagggaggaggaggccagggcccgccccacagccactctcgcgcctccgaacagccacaggggcaaagccctgtcacccccaggatccggtcatcagggaaagaggacagggagaccagaagagggccagctgggacgagggggcggacgcccaggaggcaacttctgagacgcagctcctgagaggggcagggaccaggcgcgggaggccagagggggcacagagaacaaaccccctcagaagtgaagaggagagcggaaggaaccgagaggggacggacaggagctgaggaggaaagaggaggggagaggggtcaggccaggcagccaaggagaagacgtgtggccgggggctatcagaaggaaactgggacggacgggccgggctcgggctgtcctgtggagcagcagcatccccggggccggcagaggcgccagtggctgggcgggatgagtctctgagggccactgtggagcgccccgccatggccccccgcaccctctggagctgctacctctgctgcctgctgacggcagctgcaggggccgccagctaccctcctcgaggtttcagcctctacacaggttccagtggggccctcagccccggggggccccaggcccagattgccccccggccagccagccgccacaggtaagagtctggatcccagcccgaggcttgggtggtgaggaaggggtcagaatgccacctctgcctggctctctgctgtgtccgctaatg

SEQ ID NO: 24 - DNA, 1249 bp, Artificial Sequence
  Bisulfite converted genomic DNA from chr227300945-27302193
atttagtgtgtgtttttagttttattattgatttgttgtgttattttggataagttatttttttattttgattttaatttttatatttttaaaaggagattgttagattagacggtgagtgaagttttgttgagtttttcgagtttatttttttttgtttagttagttttttttttttatttttttttttgggttttttttttggggtttttttttttaggggaggtttgttttagttttaggggagttgcggttttggtttttgtttttagtcggagtgattagttttgggttttttttttagtttcggttttggttttatgtggtttatttgagaggttggttttaagttcggcggtaattttatatttttgtttttttttttttttttttttttttcggagttaataagaagtagatgtggcgtacgatggttggagaggtggggggaggaagggggaggtcggatcgttagttagataggggggaagggaggggagttagtagggaggaggaggttagggttcgttttatagttattttcgcgttttcgaatagttataggggtaaagttttgttatttttaggattcggttattagggaaagaggatagggagattagaagagggttagttgggacgagggggcggacgtttaggaggtaatttttgagacgtagtttttgagaggggtagggattaggcgcgggaggttagagggggtatagagaataaatttttttagaagtgaagaggagagcggaaggaatcgagaggggacggataggagttgaggaggaaagaggaggggagaggggttaggttaggtagttaaggagaagacgtgtggtcgggggttattagaaggaaattgggacggacgggtcgggttcgggttgttttgtggagtagtagtattttcggggtcggtagaggcgttagtggttgggcgggatgagtttttgagggttattgtggagcgtttcgttatggtttttcgtattttttggagttgttatttttgttgtttgttgacggtagttgtaggggtcgttagttatttttttcgaggttttagtttttatataggttttagtggggtttttagtttcggggggttttaggtttagattgtttttcggttagttagtcgttataggtaagagtttggattttagttcgaggtttgggtggtgaggaaggggttagaatgttatttttgtttggttttttgttgtgttcgttaatg

SEQ ID NO: 25 - DNA, 24 bp, Artificial Sequence
  F1 Primer for Bisulfite converted genomic DNA from chr227300945-27302193
gggaggtttgttttagttttaggg

SEQ ID NO: 26 - DNA, 19 bp, Artificial Sequence
  R1 Primer for Bisulfite converted genomic DNA from chr227300945-27302193
cctaatccctacccctctc

SEQ ID NO: 27 - DNA, 22 bp, Artificial Sequence
  F2 Primer for Bisulfite converted genomic DNA from chr227300945-27302193
ttcggttattagggaaagagga

SEQ ID NO: 28 - DNA, 20 bp, Artificial Sequence
  R2 Primer for Bisulfite converted genomic DNA from chr227300945-27302193
aacgacccctacaactaccg

SEQ ID NO: 29 - DNA, 1117 bp, Homo sapiens
agtagtgggaagaagggaaaaatacagaaaacaaagggcgaaggagaaggaaaagggaagaagcaaggggaaaagaaataaaacaaaggaaatgtccaataaagaccaggaaggaaggtccgcaggtggagacgtgcgtgagggcagggagagaagggtaggaagagggaacgagagcggcacgaaggcctgggattagagagcggtgggaaaagggtagtgggcctggtgaggggagcaggagccaggccggcaaggggcaggccatcttaccgaagctgtgatgaatgattctccacccaccaggtcttcccttattgaggtggaagcggtggcgacggctggctggtccatggtggcctcctctctctctaggctgaacctgagctccggttgctaggaggcgggtgtggcccgtgggctcccgagcgcgcgcgcctgggaccgctccgggggacccgccaggtttgtctttagtgctgcagggaagggaagcgggcacgcactttgaggtcccgcgcgaagaactgcgcagaggaaaaccctcgaaaagagacggttatgaaagcttgtgagaagaaaatctagagggctctggagttgctagagagccctcgggtaaggatctgcgaaaagggaggcttgttaagggcttgagcgtacccacaatctccattagttcccttccaatccaccaccaatcgcacaccccagacagggaggcagtgtgcttggtaggcatacttaagagaaatctgccttagcacccactttctaagattgtttctaagctcctccaaatagaaactctggggctgacttctcttgcttctgacttgcagagtggagtgagcaccagacacagccggtttcactagctgtgaaaccacagaccaccccattcttctcagtatagaccctttttattgtcagcctctctaggctgtagcttcttcttaacctaggaacattctcacctttaaaaacaaaccctccacaaccttaatttcattttatacagtacctactgtattccagggacttcacacacattagtctcatttaatcttcataccaaccctacaaagtaggtattagtttcattttctggatgagg

SEQ ID NO: 30 - DNA, 1117 bp, Artificial Sequence
  Bisulfite converted genomic DNA from chr247382037-47383153
agtagtgggaagaagggaaaaatatagaaaataaagggcgaaggagaaggaaaagggaagaagtaaggggaaaagaaataaaataaaggaaatgtttaataaagattaggaaggaaggttcgtaggtggagacgtgcgtgagggtagggagagaagggtaggaagagggaacgagagcggtacgaaggtttgggattagagagcggtgggaaaagggtagtgggtttggtgaggggagtaggagttaggtcggtaaggggtaggttattttatcgaagttgtgatgaatgattttttatttattaggtttttttttattgaggtggaagcggtggcgacggttggttggtttatggtggttttttttttttttaggttgaatttgagtttcggttgttaggaggcgggtgtggttcgtgggttttcgagcgcgcgcgtttgggatcgtttcgggggattcgttaggtttgtttttagtgttgtagggaagggaagcgggtacgtattttgaggtttcgcgcgaagaattgcgtagaggaaaattttcgaaaagagacggttatgaaagtttgtgagaagaaaatttagagggttttggagttgttagagagttttcgggtaaggatttgcgaaaagggaggtttgttaagggtttgagcgtatttataatttttattagtttttttttaatttattattaatcgtatattttagatagggaggtagtgtgtttggtaggtatatttaagagaaatttgttttagtatttattttttaagattgtttttaagtttttttaaatagaaattttggggttgattttttttgtttttgatttgtagagtggagtgagtattagatatagtcggttttattagttgtgaaattatagattattttattttttttagtatagattttttttattgttagtttttttaggttgtagtttttttttaatttaggaatatttttatttttaaaaataaattttttataattttaattttattttatatagtatttattgtattttagggattttatatatattagttttatttaatttttatattaattttataaagtaggtattagttttattttttggatgagg

SEQ ID NO: 31 - DNA, 20 bp, Artificial Sequence
  F1 Primer for Bisulfite converted genomic DNA from chr247382037-47383153
gggtaggaagagggaacgag

SEQ ID NO: 32 - DNA, 22 bp, Artificial Sequence
  R1 Primer for Bisulfite converted genomic DNA from chr247382037-47383153
cccttaacaaacctcccttttc

SEQ ID NO: 33 - DNA, 22 bp, Artificial Sequence
  F2 Primer for Bisulfite converted genomic DNA from chr247382037-47383153
gaaaagggaggtttgttaaggg

SEQ ID NO: 34 - DNA, 27 bp, Artificial Sequence
  R2 Primer for Bisulfite converted genomic DNA from chr247382037-47383153
aaaataaaataatctataatttcacaa

SEQ ID NO: 35 - DNA, 931 bp, Homo sapiens
gtggggatggtcgtggctcctgtgttccccaccaaccagcaggggcacctggggaaacctccagtgcagaaatcgtactctgccatttcctgtatctccagctattccccatctgaagagtgaggtggttagaccaggtccctggttttcttccagtcaccccctgctggaatgccaggagctctcttctgtgctgggggaagctgccgctggcctcagcttccccatctgtgagatcgggctccatgcccgtcttgttggactcatcaagagacccatggggaggaaagagcttgcaaggagagcagctaaggaaagaccggaatcgtccgagccctttgtcccttcctgtatcagcagcaggcgacctgcccaccagccggggcctcccgttccgttccattccgtttaagtaagctctttccctgtgcaagggagcaaccgacctcaaaggcccggcccacaaaggggcccccgacatggctgggaacaatgcggcctgtttacctgacagattgtccgctttcttttcatctcgtaggaaaagaactcccacggggagccgctaatcccagagtaaacagatgacctcgtggtgcctttctcccggaggtggggttcctagcgcagcaccccaactgtgcaaacagaccctggagggctcgatgcgcctgcgggggcgtggctggggacctgggcctgggccaggaggggcctttccctctcgccagccctcttttcttcctttgtgatttcaattgaatggggagaagctcaaaggcaggggcatgaaaggtcaaagccaatttgcagggccaagtgtgggggaggagagaagggcccagaccaagcattctttttggaggcctgaccttgacctggaagcaactcctccctctctcggctcttctgaggacactgccctggcaga

SEQ ID NO: 36 - DNA, 931 bp, Artificial Sequence
  Bisulfite converted genomic DNA from chr2121223284-121224214
gtggggatggtcgtggtttttgtgttttttattaattagtaggggtatttggggaaatttttagtgtagaaatcgtattttgttattttttgtatttttagttattttttatttgaagagtgaggtggttagattaggtttttggtttttttttagttattttttgttggaatgttaggagtttttttttgtgttgggggaagttgtcgttggttttagtttttttatttgtgagatcgggttttatgttcgttttgttggatttattaagagatttatggggaggaaagagtttgtaaggagagtagttaaggaaagatcggaatcgttcgagttttttgtttttttttgtattagtagtaggcgatttgtttattagtcggggtttttcgtttcgttttatttcgtttaagtaagttttttttttgtgtaagggagtaatcgattttaaaggttcggtttataaaggggttttcgatatggttgggaataatgcggtttgtttatttgatagattgttcgttttttttttatttcgtaggaaaagaatttttacggggagtcgttaattttagagtaaatagatgatttcgtggtgtttttttttcggaggtggggtttttagcgtagtattttaattgtgtaaatagattttggagggttcgatgcgtttgcgggggcgtggttggggatttgggtttgggttaggaggggtttttttttttcgttagtttttttttttttttttgtgattttaattgaatggggagaagtttaaaggtaggggtatgaaaggttaaagttaatttgtagggttaagtgtgggggaggagagaagggtttagattaagtattttttttggaggtttgattttgatttggaagtaatttttttttttttcggtttttttgaggatattgttttggtaga

SEQ ID NO: 37 - DNA, 21 bp, Artificial Sequence
  F1 Primer for Bisulfite converted genomic DNA from chr2121223284-121224214
tgagatcgggttttatgttcg

SEQ ID NO: 38 - DNA, 21 bp, Artificial Sequence
  R1 Primer for Bisulfite converted genomic DNA from chr2121223284-121224214
aaacccctcctaacccaaacc

SEQ ID NO: 39 - DNA, 1275 bp, Homo sapiens
gctgctgcgggtctcggctggaatgcaatggtgcgatctccgctcactgcaacctccgcctcccgggttccagcaattctcctgcctcagcctcccaagtaggtaagattacaggagcccgccaccacgcctggctaatttttgtatttttagtagagacggggtttcaccatgttggccaggctggtctccaactcctgacctcaggtgatccacccccctcggcctcccaaagtgctgggattacaggcgtgagccacagcgcccggcagaggcaggcattataattaatctcttatacagatgaagatgcttaggtgcatgaaattaagaacttacccactatcacccacggaaaaagagaagagttggggtttgaatccacgcaagagcccacgcccttaatcattaagctaccaagatataactctgtagatgggaaaccatatctattaactggctctggaattttaaaaggcaattttataagttacaaaaatagatatggataattatttattcaatggagctgactcacaattcagaacacccaatgtgatttattctttggttggggaaacaattattgatccggtgttgattcatgttctggaaaccacatgaaaatacctaagcagccgcagatgctgagcggagctgggccaagttggtaagaactgctggtggtaggagctccagcatagcgcacagggcgggcccagagaaaggcgggcccattgttgggggattcggcttccggaggaggggacagcattcaggggtgtaccaggccccagaggcagcggaaagggagactgtggggaactaggagcaacagcaggtacactccaaattctattgacggttcgaaattcccgctttctccatagagattcctttcttcatatttcattacagtgctctgggtccattacaggcttttcttttcagacacctctctcacatccgcgggtcctcggcccccatcctttatttttgctaggccgctaaggacgctagaaagcgattcgaaactcccgctttttccatagagatgcttttcttcttatctccttacagtgctctgggtccatacaggtttttgtctcaggcacttctctcacatccgcgggtcctcggcccccatcctttatttctgctaggtccctaaggacactaggatgacggagagggagccaggggacccaggcttcaggactgagagtgaattcttgaggggaggtgaggaggaaagaggagggaccagagcgtggg

SEQ ID NO: 40 - DNA, 1275 bp, Artificial Sequence
  Bisulfite converted genomic DNA from chr542756147-42757421
gttgttgcgggtttcggttggaatgtaatggtgcgattttcgtttattgtaattttcgtttttcgggttttagtaatttttttgttttagttttttaagtaggtaagattataggagttcgttattacgtttggttaatttttgtatttttagtagagacggggttttattatgttggttaggttggtttttaatttttgattttaggtgatttatttttttcggttttttaaagtgttgggattataggcgtgagttatagcgttcggtagaggtaggtattataattaattttttatatagatgaagatgtttaggtgtatgaaattaagaatttatttattattatttacggaaaaagagaagagttggggtttgaatttacgtaagagtttacgtttttaattattaagttattaagatataattttgtagatgggaaattatatttattaattggttttggaattttaaaaggtaattttataagttataaaaatagatatggataattatttatttaatggagttgatttataatttagaatatttaatgtgatttattttttggttggggaaataattattgattcggtgttgatttatgttttggaaattatatgaaaatatttaagtagtcgtagatgttgagcggagttgggttaagttggtaagaattgttggtggtaggagttttagtatagcgtatagggcgggtttagagaaaggcgggtttattgttgggggattcggttttcggaggaggggatagtatttaggggtgtattaggttttagaggtagcggaaagggagattgtggggaattaggagtaatagtaggtatattttaaattttattgacggttcgaaattttcgttttttttatagagatttttttttttatattttattatagtgttttgggtttattataggtttttttttttagatattttttttatattcgcgggttttcggtttttattttttatttttgttaggtcgttaaggacgttagaaagcgattcgaaattttcgttttttttatagagatgtttttttttttatttttttatagtgttttgggtttatataggtttttgttttaggtattttttttatattcgcgggttttcggtttttattttttatttttgttaggtttttaaggatattaggatgacggagagggagttaggggatttaggttttaggattgagagtgaatttttgaggggaggtgaggaggaaagaggagggattagagcgtggg

SEQ ID NO: 41 - DNA, 21 bp, Artificial Sequence
  F1 Primer for Bisulfite converted genomic DNA from chr542756147-42757421
gacggggttttattatgttgg

SEQ ID NO: 42 - DNA, 21 bp, Artificial Sequence
  R1 Primer for Bisulfite converted genomic DNA from chr542756147-42757421
ctccgctcaacatctacgact

SEQ ID NO: 43 - DNA, 21 bp, Artificial Sequence
  F2 Primer for Bisulfite converted genomic DNA from chr542756147-42757421
tcggtgttgatttatgttttg

SEQ ID NO: 44 - DNA, 22 bp, Artificial Sequence
  R2 Primer for Bisulfite converted genomic DNA from chr542756147-42757421
tctccgtcatcctaatatcctt

SEQ ID NO: 45 - DNA, 771 bp, Homo sapiens
cgccctgtcgcgggagcccctgcaaatgcgtccgtggctcccgcgcctgcgcccaccaaaccactacgaggctccgccacacaactcagcagggccacgcagggcagcgcgggaaccaaagagcccagggaagtggtcaccgcccccagaggaggcaatgagacgactaactccccactgcttctactaacccaaaggtgaccacagtagaggcaaccaggatgcagtccaaagattgggatatcccactcgcttttcctggagccggatgcagtcttcccgacttccggtggagatccgggggtggggggggggcgggaggggggcccccgcggttttatgggagatgtagtttgaagcccacaggttgtgcgcaagggagaaggggtggagattgggggtggaagtgtttgggagccggcgcgggagagatcgcggtctaaaggattggagctgaatgaagggctgaaaatccttctcctgagactgtgatgtgtagatagaacgtggtgcaggcgatcggaaaactgtaggcctaaggaaacgattttaaattttccagcgtctcccacccaactcctagccctctctccaatacagtccaatgcaattgagtgctcaaagcattctttcctgttaaataatcctggactattgactagaccaagtcactcttaaacgagatattcggcctctgtaacaccttaatcattcaccccatgggaaaactggtatcatatactgacgcctaacttattcagtagtccttta

SEQ ID NO: 46 - DNA, 771 bp, Artificial Sequence
  Bisulfite converted genomic DNA from chr5102898213-102898983
cgttttgtcgcgggagtttttgtaaatgcgttcgtggttttcgcgtttgcgtttattaaattattacgaggtttcgttatataatttagtagggttacgtagggtagcgcgggaattaaagagtttagggaagtggttatcgtttttagaggaggtaatgagacgattaattttttattgtttttattaatttaaaggtgattatagtagaggtaattaggatgtagtttaaagattgggatattttattcgttttttttggagtcggatgtagttttttcgattttcggtggagattcgggggtggggggggggcgggaggggggttttcgcggttttatgggagatgtagtttgaagtttataggttgtgcgtaagggagaaggggtggagattgggggtggaagtgtttgggagtcggcgcgggagagatcgcggtttaaaggattggagttgaatgaagggttgaaaattttttttttgagattgtgatgtgtagatagaacgtggtgtaggcgatcggaaaattgtaggtttaaggaaacgattttaaattttttagcgttttttatttaatttttagttttttttttaatatagtttaatgtaattgagtgtttaaagtatttttttttgttaaataattttggattattgattagattaagttatttttaaacgagatattcggtttttgtaatattttaattatttattttatgggaaaattggtattatatattgacgtttaatttatttagtagtttttta

SEQ ID NO: 47 - DNA, 21 bp, Artificial Sequence
  F1 Primer for Bisulfite converted genomic DNA from chr5102898213-102898983
agaggaggtaatgagacgatt

SEQ ID NO: 48 - DNA, 25 bp, Artificial Sequence
  R1 Primer for Bisulfite converted genomic DNA from chr5102898213-102898983
tcgtttccttaaacctacaattttc

SEQ ID NO: 49 - DNA, 735 bp, Homo sapiens
gaggggcccagatagcaggcccagcctggggttgcgagtagtgtttcttctaacaaagctatggcaccctgagaaaggaggctcagtattcccctgtgaggatctttgtgggtctttcagttgccctaggatggtgaatatgacaataacctgagctcccaaatcatctgaagcaggacctcttaatcactcaccgctgcgctctgagccgctgggaaccaaggctggttgctaaggacgctgtgggagtcgctctgagacagacgttgctcttttacggtctcgtgaccagggcttagccaatcaggacttataagtgaatttccacctgccctgtcccgtctccaccacatttgtgttcaatttctaaagaataaatgcaatgttgtgtgaaagacctttattcgtttttaaagaacatagtaatgatttgtttcgaatattgtacatacttgcatatcctgcaagaagccacggcaagaagcggcttcacagtgctttaatgtctcctcagtgtcttaatgctgcttccccaaaacaaaccagcacgcctatcataccaaaagtagctatgtctttaaattaaattgccgttcgtaagttccttttacatagaaaatttggagcagtcagtgtcctcagcacccgtcagtgagtaaacaaagcacctagaatgttctgctatgtactaaacaccagagggcgaatttaaacacaaaagaacgcctcttttta

SEQ ID NO: 50 - DNA, 735 bp, Artificial Sequence
  Bisulfite converted genomic DNA from chr5110062134-110062868
gaggggtttagatagtaggtttagtttggggttgcgagtagtgttttttttaataaagttatggtattttgagaaaggaggtttagtatttttttgtgaggatttttgtgggttttttagttgttttaggatggtgaatatgataataatttgagtttttaaattatttgaagtaggattttttaattatttatcgttgcgttttgagtcgttgggaattaaggttggttgttaaggacgttgtgggagtcgttttgagatagacgttgtttttttacggtttcgtgattagggtttagttaattaggatttataagtgaatttttatttgttttgtttcgtttttattatatttgtgtttaatttttaaagaataaatgtaatgttgtgtgaaagatttttattcgtttttaaagaatatagtaatgatttgtttcgaatattgtatatatttgtatattttgtaagaagttacggtaagaagcggttttatagtgttttaatgtttttttagtgttttaatgttgtttttttaaaataaattagtacgtttattatattaaaagtagttatgtttttaaattaaattgtcgttcgtaagttttttttatatagaaaatttggagtagttagtgtttttagtattcgttagtgagtaaataaagtatttagaatgttttgttatgtattaaatattagagggcgaatttaaatataaaagaacgttttttttta

SEQ ID NO: 51 - DNA, 20 bp, Artificial Sequence
  F1 Primer for Bisulfite converted genomic DNA from chr5110062134-110062868
tcgttgggaattaaggttgg

SEQ ID NO: 52 - DNA, 23 bp, Artificial Sequence
  R1 Primer for Bisulfite converted genomic DNA from chr5110062134-110062868
aaaaacttacgaacgacaattta

SEQ ID NO: 53 - DNA, 1135 bp, Homo sapiens
tatgggctatttaaaaaaaacaaccaccaccaacattatagcaaaagatttcactgcatataacttatttttcatttactgcagaaaaattaaccttattggtatgactggaccaaaacttaagccatttaaaaagaagataaaacatgtctctgtcatcatcgatatatgcttttacctaaacctcaaaatccaaaatatgatggtgatttccctcataaaatgtgaattcggtagcttattttaaaagcgtgattccttgttgaatgtacaagacatataaatgcacagacttacccattcttctgagcacattggaggctgcattcagtcgcggttgttagactcaacgcagtgagtctgtaaaaggctctaacatgtaggagcctttgaccagtttcctgttttctgtgtcccaggctttccaagtaaaattgaatccaccaggacgagttgttttttccttaggatatccctctggaaagcattggtttcattttctgtgattgctctgatccctaaatcagttttgaatataagttattaaatttctccacctctaactgctcagctggttaatcaaagcttcagtctcctttccgcagccgttagttctcttggtcttaacttgttgatggcagatgggtggcttgttgcagagaagagctcctggagcagcatgagtgcatttactgaaaagcttttccgagaaacggcacaagaatggatttgcttatgtgctgcgaccacacagaactgtatataggctgacgtcaccggatgacgtgtcttttgtttgggttttccagaagctttactgttataaagcaccatgctttgtaacagcatcggggtcgttgcaggaccccctgattacaataaagcgagaaagaagaactcctgacttaaaaacatgatattccttcagtgtaggaagcagcagtgaggttgtaataaaggctgcattcaagaagacccatctgaacagtgagttgggttagcagagtgatgatttttaatgcttgtgagagccagtgttagactgccgattctgcatagagagatcatctaaatgggcagaagcctgcacaatttatgaagtgctgatttaacaccagcctatgcaaggtagtgt

SEQ ID NO: 54 - DNA, 1135 bp, Artificial Sequence
  Bisulfite converted genomic DNA from chr5138210300-138211434
tatgggttatttaaaaaaaataattattattaatattatagtaaaagattttattgtatataatttattttttatttattgtagaaaaattaattttattggtatgattggattaaaatttaagttatttaaaaagaagataaaatatgtttttgttattatcgatatatgtttttatttaaattttaaaatttaaaatatgatggtgattttttttataaaatgtgaattcggtagtttattttaaaagcgtgattttttgttgaatgtataagatatataaatgtatagatttatttatttttttgagtatattggaggttgtatttagtcgcggttgttagatttaacgtagtgagtttgtaaaaggttttaatatgtaggagtttttgattagttttttgttttttgtgttttaggttttttaagtaaaattgaatttattaggacgagttgttttttttttaggatatttttttggaaagtattggttttattttttgtgattgttttgatttttaaattagttttgaatataagttattaaatttttttatttttaattgtttagttggttaattaaagttttagtttttttttcgtagtcgttagtttttttggttttaatttgttgatggtagatgggtggtttgttgtagagaagagtttttggagtagtatgagtgtatttattgaaaagttttttcgagaaacggtataagaatggatttgtttatgtgttgcgattatatagaattgtatataggttgacgttatcggatgacgtgttttttgtttgggttttttagaagttttattgttataaagtattatgttttgtaatagtatcggggtcgttgtaggattttttgattataataaagcgagaaagaagaatttttgatttaaaaatatgatatttttttagtgtaggaagtagtagtgaggttgtaataaaggttgtatttaagaagatttatttgaatagtgagttgggttagtagagtgatgatttttaatgtttgtgagagttagtgttagattgtcgattttgtatagagagattatttaaatgggtagaagtttgtataatttatgaagtgttgatttaatattagtttatgtaaggtagtgt

SEQ ID NO: 55 - DNA, 26 bp, Artificial Sequence
  F1 Primer for Bisulfite converted genomic DNA from chr5138210300-138211434
aaaatgtgaattcggtagtttatttt

SEQ ID NO: 56 - DNA, 27 bp, Artificial Sequence
  R1 Primer for Bisulfite converted genomic DNA from chr5138210300-138211434
cccaactcactattcaaataaatcttc

SEQ ID NO: 57 - DNA, 614 bp, Homo sapiens
agtaggtaacaagataaaaggagctccctccctgatgactacagagccatcgtggcagccctgggcctccatttcagacgttcatcttgcctaagccaccaccatcagggtctcagtcagtcatcattctcatttactcgggtggctgggtgagggcggaatactaccttccagctgtctgagattaagcctaagccaccaccatcagggtctcagtcagtcatcattctcatttactcgggtggctgggcgagggcggaatactaccttccagctgtctgagattaagcagaacagcagctaaagcagtaacagcaggtctccttcagcagcttgccacagggaagagggtcccgcgtgaaccgaactcaacttccacctgcgcagaggtagctaccatttgcgtagaggtagctgggtctttaaggttagagggaaggcgcagggagggagagacggcgggtgggggttgaacaaagtggagattcacaaaagcagactagggcgggcgacgtgatcagatgacctgtgcgggcggcagcctcctgccctcctccccttcgtgcgccggctggagcgaagagttcttttgacagccgtgagcttccccgcca

SEQ ID NO: 58 - DNA, 614 bp, Artificial Sequence
  Bisulfite converted genomic DNA from chr5180257441-180258054
agtaggtaataagataaaaggagttttttttttgatgattatagagttatcgtggtagttttgggtttttattttagacgtttattttgtttaagttattattattagggttttagttagttattatttttatttattcgggtggttgggtgagggcggaatattattttttagttgtttgagattaagtttaagttattattattagggttttagttagttattatttttatttattcgggtggttgggcgagggcggaatattattttttagttgtttgagattaagtagaatagtagttaaagtagtaatagtaggttttttttagtagtttgttatagggaagagggtttcgcgtgaatcgaatttaatttttatttgcgtagaggtagttattatttgcgtagaggtagttgggtttttaaggttagagggaaggcgtagggagggagagacggcgggtgggggttgaataaagtggagatttataaaagtagattagggcgggcgacgtgattagatgatttgtgcgggcggtagttttttgtttttttttttttcgtgcgtcggttggagcgaagagtttttttgatagtcgtgagttttttcgtta

SEQ ID NO: 59 - DNA, 20 bp, Artificial Sequence
  F1 Primer for Bisulfite converted genomic DNA from chr5180257441-180258054
tcgtggtagttttgggtttt

SEQ ID NO: 60 - DNA, 21 bp, Artificial Sequence
  R1 Primer for Bisulfite converted genomic DNA from chr5180257441-180258054
ccctacgccttccctctaacc

SEQ ID NO: 61 - DNA, 1522 bp, Homo sapiens
gaagttggaggatagacacaggataaccaggcttttactggtttttggcacctttctggggtaagttgatagggactataaggagttctcctaacagaaactgacagtaacagaaactgacaggattctccggcccgtggcaattgtggaagattactgaggaagttagtactggaaataatggtcctgaaatgttatttcgttgttgctggctgctctctacaaaaaagactgagaagagagtatagagcgatagtatatgaaagagggtagggaggaatgatttaagtcttaaacattccacagtatgtaaggcttttacattataaaatgtaaacaggaccttgatgttagatgtatgataatctaatgtgaaatgtacaaaatctcccactaagaggctttataagaaatagcttagcatttattttcaatgactttaaaaatgtcctgacgggatctaagtaacacaatatttggattgttttttaaggtaatgttgagcccacagttttttacaggtagtgtgctagtatgtcctatctcattttgtaactattatttttcacattttatagatgagaaaattgagctgtagtgaagttaactatcttgctaaagatcacatagtaagtggtagaaccaggattttaacgcacattttattccagggccctagttcttaatgcttaaacttgattatgtcactgaaaatattcctatgggatataacatcagaaaagatagcgcttgtacaccacaactctaaagttggtggcgggggaaacggggagacctagcacccttctagaagattaatccaccaacatttcaaaatcttgtaaggagctgcgacccgctaggacgggagcctactgcgccctcaaaccccgcggcggctccctccccggagggggcgcgtgaggccaccacccagagcctcttcctagaggagccggaagcatcgggagccgcggtacccgcccatcttccggccaggatgtgtggtcctggtgagcgattgtcctgtggcgtccgggaccgagtttgtggcccagggttatgtgtgcacttcacacctctgacttttttcagggttcctattgatggttaggagcaggatttcggggatgaaggcggcagcagcaagaggaggctgcgactgcggaccccagattcgtccccctccaccacacacaacgccaagacgggccccaggagggcgtgaagagaagacttcctttcctctcctctcgcctcctggcgctggccgtatgaaggtgtctcccagaagcattagcagaggagccctgtgggagaaatgaggtgtatagtcacacttaaatctgtttatgctgtcattgaacctctaaacacaattggatcattttctaatgatcagaagtgttatctgtagatgtatttgtagtgagacacattagcatgcttgctttggtgtcttaattttataatatttgtagtcaagcgtcacattgtttctattgttaaacttaaaaaaagt

SEQ ID NO: 62 - DNA, 1522 bp, Artificial Sequence
  Bisulfite converted genomic DNA from chr628057937-28059458
gaagttggaggatagatataggataattaggtttttattggtttttggtatttttttggggtaagttgatagggattataaggagtttttttaatagaaattgatagtaatagaaattgataggatttttcggttcgtggtaattgtggaagattattgaggaagttagtattggaaataatggttttgaaatgttatttcgttgttgttggttgttttttataaaaaagattgagaagagagtatagagcgatagtatatgaaagagggtagggaggaatgatttaagttttaaatattttatagtatgtaaggtttttatattataaaatgtaaataggattttgatgttagatgtatgataatttaatgtgaaatgtataaaattttttattaagaggttttataagaaatagtttagtatttatttttaatgattttaaaaatgttttgacgggatttaagtaatataatatttggattgttttttaaggtaatgttgagtttatagttttttataggtagtgtgttagtatgttttattttattttgtaattattattttttatattttatagatgagaaaattgagttgtagtgaagttaattattttgttaaagattatatagtaagtggtagaattaggattttaacgtatattttattttagggttttagtttttaatgtttaaatttgattatgttattgaaaatatttttatgggatataatattagaaaagatagcgtttgtatattataattttaaagttggtggcgggggaaacggggagatttagtatttttttagaagattaatttattaatattttaaaattttgtaaggagttgcgattcgttaggacgggagtttattgcgtttttaaatttcgcggcggtttttttttcggagggggcgcgtgaggttattatttagagtttttttttagaggagtcggaagtatcgggagtcgcggtattcgtttatttttcggttaggatgtgtggttttggtgagcgattgttttgtggcgttcgggatcgagtttgtggtttagggttatgtgtgtattttatatttttgattttttttagggtttttattgatggttaggagtaggatttcggggatgaaggcggtagtagtaagaggaggttgcgattgcggattttagattcgtttttttttattatatataacgttaagacgggttttaggagggcgtgaagagaagattttttttttttttttttcgttttttggcgttggtcgtatgaaggtgttttttagaagtattagtagaggagttttgtgggagaaatgaggtgtatagttatatttaaatttgtttatgttgttattgaatttttaaatataattggattattttttaatgattagaagtgttatttgtagatgtatttgtagtgagatatattagtatgtttgttttggtgttttaattttataatatttgtagttaagcgttatattgtttttattgttaaatttaaaaaaagt

SEQ ID NO: 63 - DNA, 22 bp, Artificial Sequence
  F1 Primer for Bisulfite converted genomic DNA from chr628057937-28059458
ttcgttgttgttggttgttttt

SEQ ID NO: 64 - DNA, 22 bp, Artificial Sequence
  R1 Primer for Bisulfite converted genomic DNA from chr628057937-28059458
cgttaaaatcctaattctacca

SEQ ID NO: 65 - DNA, 26 bp, Artificial Sequence
  F2 Primer for Bisulfite converted genomic DNA from chr628057937-28059458
tgagaaaattgagttgtagtgaagtt

SEQ ID NO: 66 - DNA, 22 bp, Artificial Sequence
  R2 Primer for Bisulfite converted genomic DNA from chr628057937-28059458
cacctcatttctcccacaaaac

SEQ ID NO: 67 - DNA, 892 bp, Homo sapiens
acacattttcaaattttcagagctttccttagactaaaactttaccatcagtcctaaggtagtatgatccatgctacaaaactcgccataaaaccttactatgtaacactgctatagaaatctataaagtgtttccttcgtaggagggccgtaggcagccatggcgcccagcaggaatggcatgatgttgaagccccacttccacaaggactggcagcagcgtgtggccacgtggttcaaccagaagatccgcagaatcaaggcccggcaagccaaagggcgctgcatcgccccgcgcccggagagtcgggacccatctggcccattgtgctgtgccctgctgtgcgttatcacatcaaggtgcgcgccggcagaggcttcagcctggagctcagggtggcgggcattcacaagaaggtgacccggaccactggcatctctgtggatccgaggaggcagaacaagtccaccgattccctgcaggccaatgtgcagcgtctgaatgagtattgctccaaactcatcctcttccccagaaagccctcggcccccaagaagggagacagttctgctgaagaacagaaattggccacccagctgacaggaccggtcatgcccatcaagaatgtaaggagaaagcccgagtcatcactgagaagtagaggaattgcaaagctttcgctagtctccgcatggccggtgccaatgcttggcggcaatgctcggctcttcggcatatgggcaaaaagagccaaggaagctgaaaaacaggatgtgtgaaagcaaaaataaagccctcttggggacttgtaataaatacgttttaaaagaaatctataaagtttaaactgattcttcctctgacagagaaaggcagtttcttaacagatag

SEQ ID NO: 68 - DNA, 892 bp, Artificial Sequence
  Bisulfite converted genomic DNA from chr628829033-28829924
atatatttttaaatttttagagttttttttagattaaaattttattattagttttaaggtagtatgatttatgttataaaattcgttataaaattttattatgtaatattgttatagaaatttataaagtgtttttttcgtaggagggtcgtaggtagttatggcgtttagtaggaatggtatgatgttgaagttttatttttataaggattggtagtagcgtgtggttacgtggtttaattagaagattcgtagaattaaggttcggtaagttaaagggcgttgtatcgtttcgcgttcggagagtcgggatttatttggtttattgtgttgtgttttgttgtgcgttattatattaaggtgcgcgtcggtagaggttttagtttggagtttagggtggcgggtatttataagaaggtgattcggattattggtatttttgtggattcgaggaggtagaataagtttatcgattttttgtaggttaatgtgtagcgtttgaatgagtattgttttaaatttatttttttttttagaaagttttcggtttttaagaagggagatagttttgttgaagaatagaaattggttatttagttgataggatcggttatgtttattaagaatgtaaggagaaagttcgagttattattgagaagtagaggaattgtaaagttttcgttagttttcgtatggtcggtgttaatgtttggcggtaatgttcggtttttcggtatatgggtaaaaagagttaaggaagttgaaaaataggatgtgtgaaagtaaaaataaagtttttttggggatttgtaataaatacgttttaaaagaaatttataaagtttaaattgattttttttttgatagagaaaggtagttttttaatagatag

SEQ ID NO: 69 - DNA, 20 bp, Artificial Sequence
  F1 Primer for Bisulfite converted genomic DNA from chr628829033-28829924
agcgtgtggttacgtggttt

SEQ ID NO: 70 - DNA, 20 bp, Artificial Sequence
  R1 Primer for Bisulfite converted genomic DNA from chr628829033-28829924
ccaaacattaacaccgacca

SEQ ID NO: 71 - DNA, 1198 bp, Homo sapiens
ccggtttcccacgggttctggccttgggggaagggctattcgggccggctggtccctcaaaggcgggaagcgttgcccaggagaccaccggcctgcaggaagtgttgccctggtgacgtcaccagtgcgcgggagggacaatggggcattgttctggggtcggtgagaccgggagacagtctccccccacgagattcccccccctttccacagacactgtgttccatgccagttccccagtaagctggagcggagggccagtgtggtgttgagggtgggagttggggggggaaactcacgcggcccgtacttccaccgcatctcagattgaccgccgtaacagcaggatgagagggaatgcccctctgctgcacctatattttgcacgctatctcccaccccatctgctcaacttctctatagcatacatcaccccttcctctacataccccatttatctctggcccccacgtctgcttgggctgcaatcagttcttcaaccaggtcttccaccgccagcctggcagccagcatcaagggtgtggtcccgtcctctgtgcgagcgtccactgcagtttgtctgctacggagcagaagctggggagacagagggccagtgacccctggggtaccttggactgccaactcgagttccttacactattaaccccactcgcaatccatattcagccatcctccgcagtttccctgtcaggttcccaatcacaccaatttcctccttgtcaaactctaggggatgcttctgtccagctttacttgtaagctcgccccattccctgtagggacctcagtgtgtgctaacctggcagacctcccgagcatcagcagccacagcagcatgaaggggtgtgcgccctgcccggtctggctggttggggttggctccagcctcaaggaggcggcgggcagcggttggccgggagaatcgggcagccaggtgcaggggggtctccccagtgcccacggtgtgagcctggggacaggcccctccatccagcagaggttcccagggctcaggacatcccaaccatgccccttggaaggtcccggactgtacttccccacagcaaactgctgacatcaggggtgtcaccccatctgttggtaagacagagtaatgggtcaatctaaaggacacaacaagggggaagggacaacatgtaagctcagagagaat

SEQ ID NO: 72 - DNA, 1198 bp, Artificial Sequence
  Bisulfite converted genomic DNA from chr632164253-32165450
tcggttttttacgggttttggttttgggggaagggttattcgggtcggttggttttttaaaggcgggaagcgttgtttaggagattatcggtttgtaggaagtgttgttttggtgacgttattagtgcgcgggagggataatggggtattgttttggggtcggtgagatcgggagatagtttttttttacgagatttttttttttttttatagatattgtgttttatgttagttttttagtaagttggagcggagggttagtgtggtgttgagggtgggagttggggggggaaatttacgcggttcgtatttttatcgtattttagattgatcgtcgtaatagtaggatgagagggaatgtttttttgttgtatttatattttgtacgttattttttattttatttgtttaatttttttatagtatatattatttttttttttatatattttatttatttttggtttttacgtttgtttgggttgtaattagttttttaattaggttttttatcgttagtttggtagttagtattaagggtgtggtttcgttttttgtgcgagcgtttattgtagtttgtttgttacggagtagaagttggggagatagagggttagtgatttttggggtattttggattgttaattcgagttttttatattattaattttattcgtaatttatatttagttatttttcgtagtttttttgttaggtttttaattatattaattttttttttgttaaattttaggggatgtttttgtttagttttatttgtaagttcgttttattttttgtagggattttagtgtgtgttaatttggtagatttttcgagtattagtagttatagtagtatgaaggggtgtgcgttttgttcggtttggttggttggggttggttttagttttaaggaggcggcgggtagcggttggtcgggagaatcgggtagttaggtgtaggggggtttttttagtgtttacggtgtgagtttggggataggtttttttatttagtagaggtttttagggtttaggatattttaattatgttttttggaaggtttcggattgtatttttttatagtaaattgttgatattaggggtgttattttatttgttggtaagatagagtaatgggttaatttaaaggatataataagggggaagggataatatgtaagtttagagagaat

SEQ ID NO: 73 - DNA, 26 bp, Artificial Sequence
  F1 Primer for Bisulfite converted genomic DNA from chr632164253-32165450
agatattgtgttttatgttagttttt

SEQ ID NO: 74 - DNA, 19 bp, Artificial Sequence
  R1 Primer for Bisulfite converted genomic DNA from chr632164253-32165450
ctatccccaaactcacacc

SEQ ID NO: 75 - DNA, 731 bp, Homo sapiens
catcttcccatggaagatccttgagataatttcatggggagaagaggactttccacgcaggacatcagttgctttgctcccaaagttctctaaaagcgttcatatgtattgtgcctggaattttattatgtacatttacatgttaagttgttttcaccggcccacgttttccaccatgtgctcgtgataattgagcttccagaaggcaggtctggatagtagattaggagggtggtccccatttcacaggcggtgatgagaggctctgagcctgagccagcatcgtgaggaagtcaacaatagacggaaacacggctcgcgcccagcatcgccccaccgtgcagtgagcggcccttcctacggaaattggtgaaacacttgactttctgtttcttaaatactttaagggcataaaatcacatgtactgctttgaaagtagaaatcccagatgtattgagtcagaacttctgtaagtgagccgaggcacaggcagcatgaaaacagtcctggcgggcatcgacaggcccgggccgaagaacttctgctttacacccttccagttaacaacatatcccttatattaattgtgtgggtatgttattattctatagaaacatatataacaaattcttatgtaattatactgtgatcattaatcatataattttatgaatcgtgtatgttcttattttatagaaacttaaccaggatatacaggcgtttcattctt

SEQ ID NO: 76 - DNA, 731 bp, Artificial Sequence
  Bisulfite converted genomic DNA from chr6168393680-168394410
tatttttttatggaagatttttgagataattttatggggagaagaggattttttacgtaggatattagttgttttgtttttaaagttttttaaaagcgtttatatgtattgtgtttggaattttattatgtatatttatatgttaagttgtttttatcggtttacgttttttattatgtgttcgtgataattgagtttttagaaggtaggtttggatagtagattaggagggtggtttttattttataggcggtgatgagaggttttgagtttgagttagtatcgtgaggaagttaataatagacggaaatacggttcgcgtttagtatcgttttatcgtgtagtgagcggttttttttacggaaattggtgaaatatttgattttttgttttttaaatattttaagggtataaaattatatgtattgttttgaaagtagaaattttagatgtattgagttagaatttttgtaagtgagtcgaggtataggtagtatgaaaatagttttggcgggtatcgataggttcgggtcgaagaatttttgttttatatttttttagttaataatatattttttatattaattgtgtgggtatgttattattttatagaaatatatataataaatttttatgtaattatattgtgattattaattatataattttatgaatcgtgtatgtttttattttatagaaatttaattaggatatataggcgttttattttt

SEQ ID NO: 77 - DNA, 23 bp, Artificial Sequence
  F1 Primer for Bisulfite converted genomic DNA from chr6168393680-168394410
tggatagtagattaggagggtgg

SEQ ID NO: 78 - DNA, 20 bp, Artificial Sequence
  R1 Primer for Bisulfite converted genomic DNA from chr6168393680-168394410
aaattcttcgacccgaacct

SEQ ID NO: 79 - DNA, 656 bp, Homo sapiens
gccttccaggggccttcggatccagtgaggacccctgatcccactgccccttcatggagctcactgcagggggcacagaccccaaacagggccccggatgctggggaagaccccctggtcactgcagtccccaccagcagctcctgggcaggcagagcccaggctgtctcagcccccgagtcagggcagagccagcaacggcacgtgggcccctcatgggctgggaggccaggcgcccgccgctttcctgcggcactgttggctcacacccaaaccctgcacccagctcgtgctcggggtatctctgcggggtgggccctctgctgagtgggttcacggagtgagtcaacggagggcggtgcccagtgaggctgggattgaaccagggcaggaagcatggggggccgggagggcacccagtgggccaacacgtctttcccctgtgacaggtacgagggggtcggtgcgaacagtcaggctgggccctgtgtggttgtcacctctcattcccaccgtccgggcagatggggagactgaggcttggaggggcgggtgttggtgaaggcggctgatgtgataccgagcaggcctcattccctctctccacaacctcccgatcccaggatgctcagaggactgggtctcaggcttcccca

SEQ ID NO: 80 - DNA, 656 bp, Artificial Sequence
  Bisulfite converted genomic DNA from chr7965129-965784
gttttttaggggttttcggatttagtgaggatttttgattttattgtttttttatggagtttattgtagggggtatagattttaaatagggtttcggatgttggggaagattttttggttattgtagtttttattagtagtttttgggtaggtagagtttaggttgttttagttttcgagttagggtagagttagtaacggtacgtgggttttttatgggttgggaggttaggcgttcgtcgtttttttgcggtattgttggtttatatttaaattttgtatttagttcgtgttcggggtatttttgcggggtgggttttttgttgagtgggtttacggagtgagttaacggagggcggtgtttagtgaggttgggattgaattagggtaggaagtatggggggtcgggagggtatttagtgggttaatacgttttttttttgtgataggtacgagggggtcggtgcgaatagttaggttgggttttgtgtggttgttattttttatttttatcgttcgggtagatggggagattgaggtttggaggggcgggtgttggtgaaggcggttgatgtgatatcgagtaggttttattttttttttttataatttttcgattttaggatgtttagaggattgggttttaggttttttta

SEQ ID NO: 81 - DNA, 24 bp, Artificial Sequence
  F1 Primer for Bisulfite converted genomic DNA from chr7965129-965784
ggttaggcgttcgtcgtttttttg

SEQ ID NO: 82 - DNA, 22 bp, Artificial Sequence
  R1 Primer for Bisulfite converted genomic DNA from chr7965129-965784
tatcacatcaaccgccttcacc

SEQ ID NO: 83 - DNA, 884 bp, Homo sapiens
cgtggggttccctcccaaggacagaccaccaggggctcgggaatgggcagagtcctaccgaaaaggccatgaggctccgactggcgcctctccgtgcttcgattttggtccttgtgtttcttgttgctcctgaggctcccaaagcgcttcatgggtagctgtggccacaggagtggcgacaggtgaacatatcaggacccacagaaccggtgtcagccccaggtggccgggagccagaggagcatcagcccgacaggcacagggcgccccaaaagctcagagtggcagcctgtgctcaggtgacagcccgggttcaagcgtcctcaccagggggtcttcagggcgacctcaccgcagccccagagtctgtgaggcgccagggagtggagccgtcctgacggggctgcacctccggccacagcagctggagcgtcagcccggctgtccttaagtagcaagaagctgccggaagcgcaaacagaacttcgcaggggatttgttgatgtagttaaggtcagctgctcactgatactctccagggctttttttttttttttttttctccttaaaattaaaaaaaggagggaagaccagataggcaaaggaggcacaagcctgcaagtcatgtgcagccgtgcatgatgtctctttaaacagactgtaaaaggaaaccaccctgggcagacaattcttggcaccaattggccagagcacattcccattgtgacatgacatcacacaactattttgattcatgggttccagggccccacggggtcagaaggctccggctgctgccgggtggagaagccacacaagtccctgggctggaaattgctgaatctaccctggctcacggttccactcaatgaaagcagcaatgc

SEQ ID NO: 84 - DNA, 884 bp, Artificial Sequence
  Bisulfite converted genomic DNA from chr7151432928-151433811
cgtggggtttttttttaaggatagattattaggggttcgggaatgggtagagttttatcgaaaaggttatgaggtttcgattggcgttttttcgtgtttcgattttggtttttgtgttttttgttgtttttgaggtttttaaagcgttttatgggtagttgtggttataggagtggcgataggtgaatatattaggatttatagaatcggtgttagttttaggtggtcgggagttagaggagtattagttcgataggtatagggcgttttaaaagtttagagtggtagtttgtgtttaggtgatagttcgggtttaagcgtttttattagggggtttttagggcgattttatcgtagttttagagtttgtgaggcgttagggagtggagtcgttttgacggggttgtattttcggttatagtagttggagcgttagttcggttgtttttaagtagtaagaagttgtcggaagcgtaaatagaatttcgtaggggatttgttgatgtagttaaggttagttgtttattgatattttttagggttttttttttttttttttttttttttaaaattaaaaaaaggagggaagattagataggtaaaggaggtataagtttgtaagttatgtgtagtcgtgtatgatgtttttttaaatagattgtaaaaggaaattattttgggtagataatttttggtattaattggttagagtatatttttattgtgatatgatattatataattattttgatttatgggttttagggttttacggggttagaaggtttcggttgttgtcgggtggagaagttatataagtttttgggttggaaattgttgaatttattttggtttacggttttatttaatgaaagtagtaatgt

SEQ ID NO: 85 - DNA, 20 bp, Artificial Sequence
  F1 Primer for Bisulfite converted genomic DNA from chr7151432928-151433811
aggtggtcgggagttagagg

SEQ ID NO: 86 - DNA, 25 bp, Artificial Sequence
  R1 Primer for Bisulfite converted genomic DNA from chr7151432928-151433811
cccaaaataatttccttttacaatc

SEQ ID NO: 87 - DNA, 1327 bp, Homo sapiens
cggcgacaaaccagcaggaaaatttctccccgcggctgctctggccagcccttcagccccagcctgcgtcttgcaccggatgggtccaggggagctccgggctccgcgcggcgatgacgcccttggcaggcaaagagggaggtgcaaggggagggaacgaggagccgagctggggcgcagatgggggtggggtcgggatgctagtacgtaccgaagaggctgtggtcctgccgggtgccctgcacgctgccgtcgggcaggatctgcaggtggaagccggtgcggcaatagagctgccggcggcgcaggatgccgtgcaggtgcgccagctgcgcagcccccggcccgccgcgcgcgctccgctccgccgcgctcctgcgctcgcccagcagcggcggccgctccccggcaggaggcaacaggaaatgcgaacccacctgctggcccaagccctccaggccgcccagaaagcccccgacttcggctaagggagccatggagggggagatccggaacacaaaagacccccccagtaaagagtgttgtgggggtgggatggaggtggatagagaaaaattatagcaaaacgagcgcaaaaagttaaggcccggttactcctctgaggtcgctccggagggactttgcactgaaatggcagggaagctctcactgtcttggagcgatcttctctccttgggtaggtgggagccggctgctggctctgcagaaatatctatagctgccgctgccaatactaggactagggctgttggctggggctccaattccgcaaactgatcagatcagagtcctgtgtacatacacaccgagtatataagcgggtcctcttgacatatgctaatcaagtcctttcatgcgcgctggggaggttctgtttgaaatcttaaaccggtctcctctcaacacgtttttgctcttcggaagtgaccaaaaggggcccttggcacttctccatccatgccctcacaaaaaccattcctgcacttggccaaagccagccagcaacttcgaggaggtgcagtcgaggcccctctttggggttgtccttgggacagcggtgatgctgttcagctgcggccgtgcaaccgtctttgatgtgagctttgcagtgataaatcgcctctaaaaggagcccaacttggcagggatatagaagaagaggaagggaggagggagggaaggcggggagggagagaaagagaaggagggcattggtaggaggaggaaagagaaaaggagaattggactccttgggagcctgcagtggacaggtgcaaagaaaggtaagcaagggagaaggaaaaag

SEQ ID NO: 88 - DNA, 1327 bp, Artificial Sequence
  Bisulfite converted genomic DNA from chr816859045-16860371
cggcgataaattagtaggaaaattttttttcgcggttgttttggttagttttttagttttagtttgcgttttgtatcggatgggtttaggggagtttcgggtttcgcgcggcgatgacgtttttggtaggtaaagagggaggtgtaaggggagggaacgaggagtcgagttggggcgtagatgggggtggggtcgggatgttagtacgtatcgaagaggttgtggttttgtcgggtgttttgtacgttgtcgtcgggtaggatttgtaggtggaagtcggtgcggtaatagagttgtcggcggcgtaggatgtcgtgtaggtgcgttagttgcgtagttttcggttcgtcgcgcgcgtttcgtttcgtcgcgtttttgcgttcgtttagtagcggcggtcgtttttcggtaggaggtaataggaaatgcgaatttatttgttggtttaagttttttaggtcgtttagaaagttttcgatttcggttaagggagttatggagggggagattcggaatataaaagatttttttagtaaagagtgttgtgggggtgggatggaggtggatagagaaaaattatagtaaaacgagcgtaaaaagttaaggttcggttatttttttgaggtcgtttcggagggattttgtattgaaatggtagggaagtttttattgttttggagcgattttttttttttgggtaggtgggagtcggttgttggttttgtagaaatatttatagttgtcgttgttaatattaggattagggttgttggttggggttttaatttcgtaaattgattagattagagttttgtgtatatatatatcgagtatataagcgggtttttttgatatatgttaattaagtttttttatgcgcgttggggaggttttgtttgaaattttaaatcggtttttttttaatacgtttttgtttttcggaagtgattaaaaggggtttttggtatttttttatttatgtttttataaaaattatttttgtatttggttaaagttagttagtaatttcgaggaggtgtagtcgaggtttttttttggggttgtttttgggatagcggtgatgttgtttagttgcggtcgtgtaatcgtttttgatgtgagttttgtagtgataaatcgtttttaaaaggagtttaatttggtagggatatagaagaagaggaagggaggagggagggaaggcggggagggagagaaagagaaggagggtattggtaggaggaggaaagagaaaaggagaattggattttttgggagtttgtagtggataggtgtaaagaaaggtaagtaagggagaaggaaaaag

SEQ ID NO: 89 - DNA, 20 bp, Artificial Sequence
  F1 Primer for Bisulfite converted genomic DNA from chr816859045-16860371
tttgtcgggtgttttgtacg

SEQ ID NO: 90 - DNA, 20 bp, Artificial Sequence
  R1 Primer for Bisulfite converted genomic DNA from chr816859045-16860371
taaaaccccaaccaacaacc

SEQ ID NO: 91 - DNA, 18 bp, Artificial Sequence
  F2 Primer for Bisulfite converted genomic DNA from chr816859045-16860371
gtgggagtcggttgttgg

SEQ ID NO: 92 - DNA, 19 bp, Artificial Sequence
  R2 Primer for Bisulfite converted genomic DNA from chr816859045-16860371
accgcaactaaacaacatc

SEQ ID NO: 93 - DNA, 851 bp, Homo sapiens
gcagggggcaggaggaccagcaaaagtgaagctgtgaagaggcagggtggggcagagtgggaaaggcggggaggggtagggggcaggggtaggggagtggggggaggggaggggagggtgggggtaggggaggggagggtgcggggaggggaggggagggtgcggggaggggacgggcaaggcagagaagccagcgaggagtgaggctgccaggacatagtggggacttccccggcaccccagaccccctcgggagctgctgccaggccaggacagtgtctggcaggcacctgaagtcccgccctgcgcggggctggttcccgaggccgggttgtccaggtcgcggtctggcgtgagtcctcgtgcccttcccggctcggggattacctctctggctggtcccctcctccgtgctctgcgcgcctccaccctagcgcttgtcttggctcctagaaccaggggcctggacgctgctcagggcagaggcgccccctctgaggggctggcccctcagccgcactccgagacagccgcccccgggaccgcccttccttggagccccgccgcccgccgctcactctgcacacgcagcagaagggacgtggtgttccccaggctctggccccccaggacctgcgcggatctggcccagggcgcctcgccgacttccgtaaactgggcggagggatgaaccccgacccaggggacggaggcgctcgccctctcgctgcagggttctgccctcaacacttctggccccgcctgtgaatgggggccggagcgatggggcggggccggcctccctccctcctcccaggctgacctctgccctccttcgagcacttcccgtt

SEQ ID NO: 94 - DNA, 851 bp, Artificial Sequence
  isulfite converted genomic DNA from chr8144635010-144635860
gtagggggtaggaggattagtaaaagtgaagttgtgaagaggtagggtggggtagagtgggaaaggcggggaggggtagggggtaggggtaggggagtggggggaggggaggggagggtgggggtaggggaggggagggtgcggggaggggaggggagggtgcggggaggggacgggtaaggtagagaagttagcgaggagtgaggttgttaggatatagtggggattttttcggtattttagattttttcgggagttgttgttaggttaggatagtgtttggtaggtatttgaagtttcgttttgcgcggggttggttttcgaggtcgggttgtttaggtcgcggtttggcgtgagttttcgtgtttttttcggttcggggattatttttttggttggttttttttttcgtgttttgcgcgtttttattttagcgtttgttttggtttttagaattaggggtttggacgttgtttagggtagaggcgttttttttgaggggttggttttttagtcgtatttcgagatagtcgttttcgggatcgtttttttttggagtttcgtcgttcgtcgtttattttgtatacgtagtagaagggacgtggtgttttttaggttttggttttttaggatttgcgcggatttggtttagggcgtttcgtcgattttcgtaaattgggcggagggatgaatttcgatttaggggacggaggcgttcgttttttcgttgtagggttttgtttttaatatttttggtttcgtttgtgaatgggggtcggagcgatggggcggggtcggtttttttttttttttttaggttgatttttgttttttttcgagtatttttcgtt

SEQ ID NO: 95 - DNA, 22 bp, Artificial Sequence
  F1 Primer for Bisulfite converted genomic DNA from chr8144635010-144635860
ggggacgggtaaggtagagaag

SEQ ID NO: 96 - DNA, 20 bp, Artificial Sequence
  R1 Primer for Bisulfite converted genomic DNA from chr8144635010-144635860
aaacgccctaaaccaaatcc

SEQ ID NO: 97 - DNA, 757 bp, Homo sapiens
cttgggctaagggcggagcctaggctaggggcggagccttagtggggcggggaaggggcggggcctaagctacgggcggggcgagtcggggcggggcggggcggggccgatcggggcggggccgcgggtctcttactgtggtcgtcgcttagcgctctgtcgatgaaggctgccgccgggtagaagaagacactgaggtcgaaggtgggcaggtcgtcggcctccacgccgtggtactggatgtccatgtcgcggtagtagtcgggcccagtgtccacgttccagcggccgtgggccgcgttcagcacgtgcgtgaaccccgccttctgcagcctatagcggtccagcgccgtcgccctgggcgcaggggagagaaatctgtgggcgcgcggcgccctgccccgggtccgggaaactcaggggccacccacgctttccgctgtggattccagttccagaatgaggactagttattgcaaatgaagtcccaaagctctgctcttacccggtcccctaggccacctgcactgttttgtgattctggctcggctgtctcaagaaaggacagggtggaggctggggtaggcaagctgagctattcgttcgtccattagttctttcatcattcagtatacacagcggaacctggtgctggcctctacggtggtcgcagatctggacctgccaaatcgcctgaaagccctccagaataacctgttcatcagggctgccttctcccaacctctgctctgaacct

SEQ ID NO: 98 - DNA, 757 bp, Artificial Sequence
  Bisulfite converted genomic DNA from chr1076803419-76804175
tttgggttaagggcggagtttaggttaggggcggagttttagtggggcggggaaggggcggggtttaagttacgggcggggcgagtcggggcggggcggggcggggtcgatcggggcggggtcgcgggttttttattgtggtcgtcgtttagcgttttgtcgatgaaggttgtcgtcgggtagaagaagatattgaggtcgaaggtgggtaggtcgtcggtttttacgtcgtggtattggatgtttatgtcgcggtagtagtcgggtttagtgtttacgttttagcggtcgtgggtcgcgtttagtacgtgcgtgaatttcgttttttgtagtttatagcggtttagcgtcgtcgttttgggcgtaggggagagaaatttgtgggcgcgcggcgttttgtttcgggttcgggaaatttaggggttatttacgtttttcgttgtggattttagttttagaatgaggattagttattgtaaatgaagttttaaagttttgtttttattcggttttttaggttatttgtattgttttgtgattttggttcggttgttttaagaaaggatagggtggaggttggggtaggtaagttgagttattcgttcgtttattagtttttttattatttagtatatatagcggaatttggtgttggtttttacggtggtcgtagatttggatttgttaaatcgtttgaaagttttttagaataatttgtttattagggttgtttttttttaatttttgttttgaattt

SEQ ID NO: 99 - DNA, 20 bp, Artificial Sequence
  F1 Primer for Bisulfite converted genomic DNA from chr1076803419-76804175
gtcggtttttacgtcgtggt

SEQ ID NO: 100 - DNA, 22 bp, Artificial Sequence
  R1 Primer for Bisulfite converted genomic DNA from chr1076803419-76804175
ccaacctccaccctatcctttc

SEQ ID NO: 101 - DNA, 662 bp, Homo sapiens
ggcaggagggccactctgacggccattgtgtgaaggccccatcgttgatgttgggaagcactgtgactggctgcccagggacccaggttccgctttggggagatccacctgctacaaggagggcagtgctgggacgtcactcagcactaagggcccactagcgtttgggatgtcgtggggagggggctgtgtccccggatctcccaccagggccaggacctccctgtggtctctcggtgcaggtggaggacgccatgctggacacctacgacctggtatatgagcaggcgatgaaaggtacgtcccacgtccggcggcaggagctggcggccatccaggacgtggtgagcgtggggacggctgggtggcagggcggtcagcttctgcttggactgcagttcagagaacaggcgcagggtggccagtgagaggtctggccaggcaccgagggggttccaggacacaggccagagttgcccctcagggctgggggcaaaaagctcccaccctctgtctgcccaggacaaggccgcctaccagattctcgaggcccagtgcaaaacgagagggcagggccctgtattcagaaacactgaaggatttcaagagcattaaagcaaatacggggccgaacatagtggctcacacctgtaatcccagca

SEQ ID NO: 102 - DNA, 662 bp, Artificial Sequence
  Bisulfite converted genomic DNA from chr112334642-2335303
ggtaggagggttattttgacggttattgtgtgaaggttttatcgttgatgttgggaagtattgtgattggttgtttagggatttaggtttcgttttggggagatttatttgttataaggagggtagtgttgggacgttatttagtattaagggtttattagcgtttgggatgtcgtggggagggggttgtgttttcggattttttattagggttaggatttttttgtggtttttcggtgtaggtggaggacgttatgttggatatttacgatttggtatatgagtaggcgatgaaaggtacgttttacgttcggcggtaggagttggcggttatttaggacgtggtgagcgtggggacggttgggtggtagggcggttagtttttgtttggattgtagtttagagaataggcgtagggtggttagtgagaggtttggttaggtatcgagggggttttaggatataggttagagttgttttttagggttgggggtaaaaagtttttattttttgtttgtttaggataaggtcgtttattagattttcgaggtttagtgtaaaacgagagggtagggttttgtatttagaaatattgaaggattttaagagtattaaagtaaatacggggtcgaatatagtggtttatatttgtaattttagta

SEQ ID NO: 103 - DNA, 20 bp, Artificial Sequence
  F1 Primer for Bisulfite converted genomic DNA from chr112334642-2335303
attagcgtttgggatgtcgt

SEQ ID NO: 104 - DNA, 20 bp, Artificial Sequence
  R1 Primer for Bisulfite converted genomic DNA from chr112334642-2335303
cctaaaaccccctcgatacc

SEQ ID NO: 105 - DNA, 542 bp, Homo sapiens
cgagggccagcggagccgtgctgcgcgcagggatggacgctgggcgcccaaggcccgttcccgtggacaggcgcgcggagggcagcaggaccgatgcaccgacggacgcaccgacggacgcgcggacagatgcgccgaccgcaggctctcccgccgtcccggggccgtcgtgcccgtgcaggccgcgagcagcggcggggccggctctgcggctgcagcagccccattgtgaggccggcgagacaatgggcggcccgaggaggcacctgctcgcctgaaaggcccataaatcgccgccgcgtccagctgccttcccgcccctccccgcggacccgctagcgggaagcgcggccgcggccgggagggggaggctcggtccgacagtcgggcagggccgcctgggccaccgccgcgcgctccctcccgcctgggcgcagctgcccgcacgccccgacgggagctgcgcccagcctcccctctggagtcggcgctccaggctgctgcccggccggtggccaaggccccagcaccaccgtccctcc

SEQ ID NO: 106 - DNA, 542 bp, Artificial Sequence
  Bisulfite converted genomic DNA from chr112890827-2891368
cgagggttagcggagtcgtgttgcgcgtagggatggacgttgggcgtttaaggttcgttttcgtggataggcgcgcggagggtagtaggatcgatgtatcgacggacgtatcgacggacgcgcggatagatgcgtcgatcgtaggttttttcgtcgtttcggggtcgtcgtgttcgtgtaggtcgcgagtagcggcggggtcggttttgcggttgtagtagttttattgtgaggtcggcgagataatgggcggttcgaggaggtatttgttcgtttgaaaggtttataaatcgtcgtcgcgtttagttgttttttcgttttttttcgcggattcgttagcgggaagcgcggtcgcggtcgggagggggaggttcggttcgatagtcgggtagggtcgtttgggttatcgtcgcgcgttttttttcgtttgggcgtagttgttcgtacgtttcgacgggagttgcgtttagttttttttttggagtcggcgttttaggttgttgttcggtcggtggttaaggttttagtattatcgttttttt

SEQ ID NO: 107 - DNA, 20 bp, Artificial Sequence
  F1 Primer for Bisulfite converted genomic DNA from chr112890827-2891368
tcgatgtatcgacggacgta

SEQ ID NO: 108 - DNA, 20 bp, Artificial Sequence
  R1 Primer for Bisulfite converted genomic DNA from chr112890827-2891368
caaacgaccctacccgacta

SEQ ID NO: 109 - DNA, 2178 bp, Homo sapiens
tgctgtacagaagtctccagagaaccttcttctctgatttattcagagcctgtttgagaacagggtcaaagaggattaagaaggatccagaagacagccaggtttctccaggcaagagaactacagggcagttgctctgctgggcttactagggcaggtttctaagagggttccgaaagcctcttaagaacctgtgttaaattggcagccacagaaaccactagctccagctgacagctcaccctgggcacgatgccagttcagagagagaactgaaacgatatcaaagccgaagttttcttctttctgccactctcctgagcacagcagataaattttggaataagaagcttctattaatatttgccagcaagtgaattttcttcaaaacttaaaaaggaggtgaagaaatcaaatcctctaatacagaaaatttgtgaatacaagagtgagaagcataatctttatggttgttaatgtaatttaaattttaaaacaatgaaaatatctaggtaattgaaacaaaagactgaaataagtaaataactttgcttcagatttccttttataagctttgaacaaatgcaggtatatttaatgacatatagaaaacaggcaaaatttattttctgacaacaggagttatgaaaatgattcttttctaaaattcttttgatgtaaaactttattttaaaattaattaactgaacaaacatagtaagttcatgagattgtccagtccctagaaagggctatacttaatacttcacataaaaatggattttctcaaaggcaagtggtttaaacaatacccttaacccattgataccaaagtctaaataaatatccttaaaaatgaggatgcagggggctgggaatttggtatcttctttctgaaatatgagtttctagatatacagagacttaccactgaacgggtttgcctgttaagttgcataagactctggatatttaggatataaaaaagacttaatagaacagatcagtttctgcggagactgggaaatgtgaaagtaatggtactttaagattataacagagaaagggggaagaagaggaggagggagaggaaaaatagaagtaagccaaagtcagaggcagaaaagtaaacacagagaaatgggagaaaagtttctcaccataacacaaagttactaccgctaaggcggccaggatggcaagaaggatcaggctcttcatggtttcgtcctgcaggtcagtctcagggtcttgtgtagcagcagtagggagagaggctcctacgggatgttagtggaaggctgtgaggtttttataagaaccagagcagttgagaggggaggaaccagtgggagagagctggagaaggtggggaagggttggcactgaactagcattggaacttttcccaaacagtcattccttccagatattatctactctgagtgggcagttttccctaactgctggacctgactgtgttacacaggatgctgctctggtgcagaagttttggccatcgtatgcttggggacagacctgggcaaaagcccacagaggaagttgccacaaacacatgatctaccctcctggccctgaccgcagggcttttgggtttggtccacagacagagccctagtgttctgtttgttaccctgattgattgatgagaggttttgggggagaaaggacttcactttcttttcttttcttctttttaacctttgcctcttcttctaggagaacttcgctttctacactgattataatttagacatcttcccagtagggctgaatcctagaccaatctatcaatcccagactaatcaggcatttgcctggggatatgcatctttggcatttttccaagggttcatcaggatggagatatccggtgcaccatgagttctgtttccttaatcaacaccgttgtaacttgcccatccagttttgtgacattaattcaaacctgtgccctagtcctcttttaggcagcgtatcagtgctggaaagtgcagcaaggataagagggtactgttctctcatttctgagggcgttgtctcgataattaactaacttgatagactttttagtgagtggcaggtgagatgcaaggtactgtgctaggtgctgtgggggatgtacagacaaacaacacacctccctaaggaggtaag

SEQ ID NO: 110 - DNA, 2178 bp, Artificial Sequence
  Bisulfite converted genomic DNA from chr1215037505-15039682
tgttgtatagaagtttttagagaatttttttttttgatttatttagagtttgtttgagaatagggttaaagaggattaagaaggatttagaagatagttaggtttttttaggtaagagaattatagggtagttgttttgttgggtttattagggtaggtttttaagagggtttcgaaagttttttaagaatttgtgttaaattggtagttatagaaattattagttttagttgatagtttattttgggtacgatgttagtttagagagagaattgaaacgatattaaagtcgaagtttttttttttttgttatttttttgagtatagtagataaattttggaataagaagtttttattaatatttgttagtaagtgaattttttttaaaatttaaaaaggaggtgaagaaattaaattttttaatatagaaaatttgtgaatataagagtgagaagtataatttttatggttgttaatgtaatttaaattttaaaataatgaaaatatttaggtaattgaaataaaagattgaaataagtaaataattttgttttagatttttttttataagttttgaataaatgtaggtatatttaatgatatatagaaaataggtaaaatttattttttgataataggagttatgaaaatgatttttttttaaaatttttttgatgtaaaattttattttaaaattaattaattgaataaatatagtaagtttatgagattgtttagtttttagaaagggttatatttaatattttatataaaaatggatttttttaaaggtaagtggtttaaataatatttttaatttattgatattaaagtttaaataaatatttttaaaaatgaggatgtagggggttgggaatttggtattttttttttgaaatatgagtttttagatatatagagatttattattgaacgggtttgtttgttaagttgtataagattttggatatttaggatataaaaaagatttaatagaatagattagtttttgcggagattgggaaatgtgaaagtaatggtattttaagattataatagagaaagggggaagaagaggaggagggagaggaaaaatagaagtaagttaaagttagaggtagaaaagtaaatatagagaaatgggagaaaagttttttattataatataaagttattatcgttaaggcggttaggatggtaagaaggattaggttttttatggtttcgttttgtaggttagttttagggttttgtgtagtagtagtagggagagaggtttttacgggatgttagtggaaggttgtgaggtttttataagaattagagtagttgagaggggaggaattagtgggagagagttggagaaggtggggaagggttggtattgaattagtattggaattttttttaaatagttattttttttagatattatttattttgagtgggtagttttttttaattgttggatttgattgtgttatataggatgttgttttggtgtagaagttttggttatcgtatgtttggggatagatttgggtaaaagtttatagaggaagttgttataaatatatgatttatttttttggttttgatcgtagggtttttgggtttggtttatagatagagttttagtgttttgtttgttattttgattgattgatgagaggttttgggggagaaaggattttatttttttttttttttttttttttaatttttgtttttttttttaggagaatttcgttttttatattgattataatttagatatttttttagtagggttgaattttagattaatttattaattttagattaattaggtatttgtttggggatatgtatttttggtatttttttaagggtttattaggatggagatattcggtgtattatgagttttgtttttttaattaatatcgttgtaatttgtttatttagttttgtgatattaatttaaatttgtgttttagtttttttttaggtagcgtattagtgttggaaagtgtagtaaggataagagggtattgtttttttatttttgagggcgttgtttcgataattaattaatttgatagattttttagtgagtggtaggtgagatgtaaggtattgtgttaggtgttgtgggggatgtatagataaataatatatttttttaaggaggtaag

SEQ ID NO: 111 - DNA, 25 bp, Artificial Sequence
  F1 Primer for Bisulfite converted genomic DNA from chr1215037505-15039682
tttttaagaatttgtgttaaattgg

SEQ ID NO: 112 - DNA, 21 bp, Artificial Sequence
  R1 Primer for Bisulfite converted genomic DNA from chr1215037505-15039682
aattcccaaccccctacatcc

SEQ ID NO: 113 - DNA, 24 bp, Artificial Sequence
  F2 Primer for Bisulfite converted genomic DNA from chr1215037505-15039682
ttaaaggtaagtggtttaaataat

SEQ ID NO: 114 - DNA, 20 bp, Artificial Sequence
  R2 Primer for Bisulfite converted genomic DNA from chr1215037505-15039682
cttccccaccttctccaact

SEQ ID NO: 115 - DNA, 19 bp, Artificial Sequence
  F3 Primer for Bisulfite converted genomic DNA from chr1215037505-15039682
gggaggaattagtgggaga

SEQ ID NO: 116 - DNA, 26 bp, Artificial Sequence
  R3 Primer for Bisulfite converted genomic DNA from chr1215037505-15039682
ccctcttatccttactacactttcca

SEQ ID NO: 117 - DNA, 1516 bp, Homo sapiens
accgggcccacgccagcggaattgcgcatgcgcagggccgcctctgcctgcggcctgggctgggtgtagccccggaacctagctcttaactcccgccagcagttttacgcatgcgcgacgacgcctaggcctgtggcacggtcgcggctctagtcccctgggcccctcagctgtctctggcggggaggtcgggccgcaggggcggggaggggtggcctgccccagtggtacctgacagcatcccacgtcacggccgcgatccccctcggcctcccacaccactcctccaccctcttacctcctcgcaggccggagccttgtccgctccgcctcctgggtagcggttactgcacggtcaggggcccggcgccgcgatgagttggcgcctccccctgaaacggcccggcctcggcgggatgcgctcgggtgcccggccgagacggaaacgaagggagcgcagacaagaggagggggctggcgacggcgtctcgcccctcctctcctccctaggctgcccaccgccgcccctcccctttctcgcagacgccccctcccctcggagttgacccccgggagtctcccattggggccgcctccgcttttccagggagttcctccgtgggtcacccccaccttctccagggttatgttctcgcaaattttgggaatttgtggatacccgacctagaaggataaggcgtgtatgatgctgagagcaaccttttccacagcccctctcctccagcctgccccagcttttcttctattgctggcccctctatctgggggctccgatcacttcacgttttcaccccattgctgtggggcggaagtcttgtagccccctgtagtacaaatgctgaaataatgaggatgtgtgctggggggttcagggggatgacctcagttaccttggggccaagggcttcgaaaacagccaccttctccccttatgattatattttgcagtaatttattatattacactgcacttctgttggcctttgtagatttaagtaaaatgcaccacaactttgcagtgtgcaataatttcttccactgcactacacttaaattgacctgagtgggttttggttaaatgctcaacaattaaatgttattagctattcctgtgaagtaagctacttgcttttatactggctgtttcatgaggacactggttcctctacagtgtagagccaaaagtagtaaagccaaagagaaaaactaacattccaaaccacagtgaccgtgtcagtctgtttcgtctggcttttgtcatttcttcggaaaaacatcattctaaaatgtataactgcctttctgctacatagggcagacatttatctctattctctgagcatagcccactgagcctcagaaggaggcacgcaaaatggtacacaggtatttcttgtagagctctaaatggaagaaaaagtaagtatacattaacaggaagttgcagcgatagtgcagagaggcatcgtgtgcctgccccaagtttctccactgg

SEQ ID NO: 118 - DNA, 1516 bp, Artificial Sequence
  Bisulfite converted genomic DNA from chr1349106866-49108381
atcgggtttacgttagcggaattgcgtatgcgtagggtcgtttttgtttgcggtttgggttgggtgtagtttcggaatttagtttttaattttcgttagtagttttacgtatgcgcgacgacgtttaggtttgtggtacggtcgcggttttagttttttgggttttttagttgtttttggcggggaggtcgggtcgtaggggcggggaggggtggtttgttttagtggtatttgatagtattttacgttacggtcgcgatttttttcggttttttatattatttttttatttttttattttttcgtaggtcggagttttgttcgtttcgttttttgggtagcggttattgtacggttaggggttcggcgtcgcgatgagttggcgtttttttttgaaacggttcggtttcggcgggatgcgttcgggtgttcggtcgagacggaaacgaagggagcgtagataagaggagggggttggcgacggcgtttcgtttttttttttttttttaggttgtttatcgtcgttttttttttttttcgtagacgtttttttttttcggagttgattttcgggagttttttattggggtcgttttcgtttttttagggagttttttcgtgggttatttttattttttttagggttatgttttcgtaaattttgggaatttgtggatattcgatttagaaggataaggcgtgtatgatgttgagagtaattttttttatagttttttttttttagtttgttttagttttttttttattgttggtttttttatttgggggtttcgattattttacgtttttattttattgttgtggggcggaagttttgtagttttttgtagtataaatgttgaaataatgaggatgtgtgttggggggtttagggggatgattttagttattttggggttaagggtttcgaaaatagttattttttttttttatgattatattttgtagtaatttattatattatattgtatttttgttggtttttgtagatttaagtaaaatgtattataattttgtagtgtgtaataattttttttattgtattatatttaaattgatttgagtgggttttggttaaatgtttaataattaaatgttattagttatttttgtgaagtaagttatttgtttttatattggttgttttatgaggatattggtttttttatagtgtagagttaaaagtagtaaagttaaagagaaaaattaatattttaaattatagtgatcgtgttagtttgtttcgtttggtttttgttattttttcggaaaaatattattttaaaatgtataattgtttttttgttatatagggtagatatttatttttattttttgagtatagtttattgagttttagaaggaggtacgtaaaatggtatataggtattttttgtagagttttaaatggaagaaaaagtaagtatatattaataggaagttgtagcgatagtgtagagaggtatcgtgtgtttgttttaagtttttttattgg

SEQ ID NO: 119 - DNA, 21 bp, Artificial Sequence
  F1 Primer for Bisulfite converted genomic DNA from chr1349106866-49108381
gggaggggtggtttgttttag

SEQ ID NO: 120 - DNA, 21 bp, Artificial Sequence
  R1 Primer for Bisulfite converted genomic DNA from chr1349106866-49108381
aaaataatcgaaacccccaaa

SEQ ID NO: 121 - DNA, 21 bp, Artificial Sequence
  F2 Primer for Bisulfite converted genomic DNA from chr1349106866-49108381
gggggtttcgattattttacg

SEQ ID NO: 122 - DNA, 26 bp, Artificial Sequence
  R2 Primer for Bisulfite converted genomic DNA from chr1349106866-49108381
aaaactcaataaactatactcaaaaa

SEQ ID NO: 123 - DNA, 1157 bp, Homo sapiens
tgttgttgttgttgaggttggggccgtctccgctcgccggcccgggcggcccgtgccgcggcgggctcgccatcgcgggcgcttcaggccgcgcggcccgggcccggcgcccaggggttggggcgaggggcggagggggcgcgggcgggggcggctccctcccacccttgcgcccggccgcccgcccgatcacgcgtccctcgggcccaggcggtggggaaggtccggggaggcccgcgggggccagcaccgctcggcggcgccgcgccctccgcgctctggggctcctgaggatgcccggcgcgggcggtggccgcccccctccccggctgcctgcggccgctgcctcctcgggctctcgggcggcgccgggggacgcgctcgctgggccgggagtcggggtccccgagccgcggggccgagcctaaggcgcgcgcggccgcaccggggctgctgccgccgcgtcgcgctccgggaagccggggtgcgcccgggcgctcggggtccgcgccgccgccggggctgctgctgctgctggtgttgctgctgctgctgccaacggcgacccgggctcgtcgcggtccccgccgcacagtgagtaacacatcgcgcaccgagtgactgaactaagaagagcaaaacaacatgtgactcggcgttacgcaggcacacacagcgcggccgccccgccccgctgcctcgcattggcgccgcgccccccgacggacggcgcgctcggccaatcggcgcggcgctcgcgggggcgggccgcgcgcccccgccccgcccccctttctccccgggccgcgtttcccgccgtcccctccccctcccgcgaaggccccggcccggccgggcggggtggggcggccccggcctcattaatcagcggcttgttgtggatgccggcggaggagatgccacccagggcgggaaaaggggcgcggaagaggggcggggcgggccacggcgcgcagggcccttccctcccgcctcggactcaattaattgggcttgagcttccgccggggagggggcgccgggcggggccgcggctgggcggggcgggggatcgggatcggcggcgggggctgcggccttgcagtggaagcatgggcggcgagccgggccgtgctctcggggcgcggggtccccat

SEQ ID NO: 124 - DNA, 1157 bp, Artificial Sequence
  Bisulfite converted genomic DNA from chr13110438328-110439484
tgttgttgttgttgaggttggggtcgttttcgttcgtcggttcgggcggttcgtgtcgcggcgggttcgttatcgcgggcgttttaggtcgcgcggttcgggttcggcgtttaggggttggggcgaggggcggagggggcgcgggcgggggcggtttttttttatttttgcgttcggtcgttcgttcgattacgcgtttttcgggtttaggcggtggggaaggttcggggaggttcgcgggggttagtatcgttcggcggcgtcgcgtttttcgcgttttggggtttttgaggatgttcggcgcgggcggtggtcgttttttttttcggttgtttgcggtcgttgttttttcgggttttcgggcggcgtcgggggacgcgttcgttgggtcgggagtcggggttttcgagtcgcggggtcgagtttaaggcgcgcgcggtcgtatcggggttgttgtcgtcgcgtcgcgtttcgggaagtcggggtgcgttcgggcgttcggggttcgcgtcgtcgtcggggttgttgttgttgttggtgttgttgttgttgttgttaacggcgattcgggttcgtcgcggttttcgtcgtatagtgagtaatatatcgcgtatcgagtgattgaattaagaagagtaaaataatatgtgattcggcgttacgtaggtatatatagcgcggtcgtttcgtttcgttgtttcgtattggcgtcgcgtttttcgacggacggcgcgttcggttaatcggcgcggcgttcgcgggggcgggtcgcgcgttttcgtttcgttttttttttttttcgggtcgcgtttttcgtcgttttttttttttttcgcgaaggtttcggttcggtcgggcggggtggggcggtttcggttttattaattagcggtttgttgtggatgtcggcggaggagatgttatttagggcgggaaaaggggcgcggaagaggggcggggcgggttacggcgcgtagggtttttttttttcgtttcggatttaattaattgggtttgagttttcgtcggggagggggcgtcgggcggggtcgcggttgggcggggcgggggatcgggatcggcggcgggggttgcggttttgtagtggaagtatgggcggcgagtcgggtcgtgttttcggggcgcggggtttttat

SEQ ID NO: 125 - DNA, 19 bp, Artificial Sequence
  F1 Primer for Bisulfite converted genomic DNA from chr13110438328-110439484
ggttcgcgggggttagtat

SEQ ID NO: 126 - DNA, 20 bp, Artificial Sequence
  R1 Primer for Bisulfite converted genomic DNA from chr13110438328-110439484
ccctaaataacatctcctcc

SEQ ID NO: 127 - DNA, 520 bp, Homo sapiens
gttgtctttctcaacataatctcagaatttcagtgttcagagctgctatctatacacgggaaaacaagttttaaaattcagggacagagaaaaatagaaaaataaagataaacaatagaattagacaaactagacgtttctttcattgatgatgcaggttttcttccttttttattttttatttttttattatactttaagttctagggtacgaggccagggagaaggagaagccaccctgaggaaggtgcggaatgtcgcgtggagcccggctctctgcctttgaagcaggattttcatgcactcgccagcatggctggcttttcagactggccagatttaactcgggaccgttgtatagaggatcaggtttgagcctgccttccaaagaaagcagaagtctccagaaacaaagcctcccagatccagcttacaggctgatggctgtgaaggaagaatttgaagggcaattaagtgtgctgtgaaacatagcaagaaaggcattaattcctcactctct

SEQ ID NO: 128 - DNA, 520 bp, Artificial Sequence
  Bisulfite converted genomic DNA from chr13112861249-112861768
gttgttttttttaatataattttagaattttagtgtttagagttgttatttatatacgggaaaataagttttaaaatttagggatagagaaaaatagaaaaataaagataaataatagaattagataaattagacgttttttttattgatgatgtaggtttttttttttttttattttttatttttttattatattttaagttttagggtacgaggttagggagaaggagaagttattttgaggaaggtgcggaatgtcgcgtggagttcggttttttgtttttgaagtaggatttttatgtattcgttagtatggttggttttttagattggttagatttaattcgggatcgttgtatagaggattaggtttgagtttgttttttaaagaaagtagaagtttttagaaataaagttttttagatttagtttataggttgatggttgtgaaggaagaatttgaagggtaattaagtgtgttgtgaaatatagtaagaaaggtattaattttttatttttt

SEQ ID NO: 129 - DNA, 23 bp, Artificial Sequence
  F1 Primer for Bisulfite converted genomic DNA from chr13112861249-112861768
agggatagagaaaaatagaaaaa

SEQ ID NO: 130 - DNA, 21 bp, Artificial Sequence
  R1 Primer for Bisulfite converted genomic DNA from chr13112861249-112861768
cttccttcacaaccatcaacc

SEQ ID NO: 131 - DNA, 716 bp, Homo sapiens
agcccagccagagctcgcctcaccctgcattctgcaggcagcatgcattttttttttttttttttgaaacacccctacatgttccagcctttaaatagcagcgagtcttctaaagtgatgctgtgtgctgcaagagaaatgaaagcgcctgccttggaggcaggaagccgactagggagatgcttaattatcgtggaagtaggtgcttgttaccattgagtggagagtgcagcaagtgcaggtcgtcctccggtgtcagagccgtgatttgtagacaatgccgccctgtcagctcagtgctggttcagcagcatctttccaaacatgtatttggcaagaatttaaaggccacctcttgtgtaatttgtggagtcttggaaaaggagcctctgcctttcttttcagaatgtacgcaaccatgcagaattttgtactcagaatctgtgtctcctgccttctgcctgacgtgctcatgaggaagtttgtttccatgcccgtcacctggaaggatgctaacagttctctgtaatctgttggggatatggagagggccacccagagctgtgttttgtttctgccgtcttcataaattagcactgatggggtggagtaggttgtactgcatgaggaaatgcaggcattttgttcattagtgagatacttctagcttttaagagtacaagaaactaaggagagcttgggaagaggtggtggtg

SEQ ID NO: 132 - DNA, 716 bp, Artificial Sequence
  Bisulfite converted genomic DNA from chr1472052896-72053611
agtttagttagagttcgttttattttgtattttgtaggtagtatgtattttttttttttttttttgaaatatttttatatgttttagtttttaaatagtagcgagttttttaaagtgatgttgtgtgttgtaagagaaatgaaagcgtttgttttggaggtaggaagtcgattagggagatgtttaattatcgtggaagtaggtgtttgttattattgagtggagagtgtagtaagtgtaggtcgtttttcggtgttagagtcgtgatttgtagataatgtcgttttgttagtttagtgttggtttagtagtatttttttaaatatgtatttggtaagaatttaaaggttattttttgtgtaatttgtggagttttggaaaaggagtttttgtttttttttttagaatgtacgtaattatgtagaattttgtatttagaatttgtgttttttgttttttgtttgacgtgtttatgaggaagtttgtttttatgttcgttatttggaaggatgttaatagttttttgtaatttgttggggatatggagagggttatttagagttgtgttttgtttttgtcgtttttataaattagtattgatggggtggagtaggttgtattgtatgaggaaatgtaggtattttgtttattagtgagatatttttagtttttaagagtataagaaattaaggagagtttgggaagaggtggtggtg

SEQ ID NO: 133 - DNA, 22 bp, Artificial Sequence
  F1 Primer for Bisulfite converted genomic DNA from chr1472052896-72053611
ggaggtaggaagtcgattaggg

SEQ ID NO: 134 - DNA, 20 bp, Artificial Sequence
  R1 Primer for Bisulfite converted genomic DNA from chr1472052896-72053611
accctctccatatccccaac

SEQ ID NO: 135 - DNA, 902 bp, Homo sapiens
tcagccatcagggccgcctgccctcccctataaccctgggtggtcctccgtctggcgcattcccccgtatcctgcaggggtctgcctggcgttactttctccgagaccttcctgaccttcttcttgcccctctccccccatattaggtgcaaggatgtcctggtacgtctgacccacgaacgcaaatcccttctctggtttgccttccatctcctcaccctgaggtgctcaagaaacgtatgtagggtcccggtccgctcaaagcgcacaaacctccgaggcagcagcctctaaatagagaggccagggagtgaccggagggcattttcttgtacacacaggggttgccacggaactcccgtgaaacagactcccgggccagccgcggcgctcaagcttcccctgaggtcctagaaaggctcaccctgggctcgctcggggcggcggctcccggcaggcctagcgcggcggggcggggccggcggcacgaggaccaggcagcgcgggcagcacgcgcccgggccggagcccgcccacaagggcgcgcggcgcgttctgattggaaggcctcgagcgggggcggggcttccagggtgcgcttccgccgtcgcctgttcccgccgcggagacccggcagttgggggatgccgacgcctgggccttgaggatgctgcggaagctcaccatcgagcagatcaacgactggttcaccatcggcaagacggtgaccaatgtggagctgctgggcgcgccgcccgccttcccggcaggggcggccagggaggaggtgcagcgccaggacgtggcccccggcgctggtcccgcggcccaggctccggctctggcccaagctccggcccggccggccgctgcgttcgaaaggtaggacgcgggcggggcgg

SEQ ID NO: 136 - DNA, 902 bp, Artificial Sequence
  Bisulfite converted genomic DNA from chr14104394180-104395081
ttagttattagggtcgtttgtttttttttataattttgggtggtttttcgtttggcgtattttttcgtattttgtaggggtttgtttggcgttatttttttcgagatttttttgattttttttttgtttttttttttttatattaggtgtaaggatgttttggtacgtttgatttacgaacgtaaattttttttttggtttgttttttatttttttattttgaggtgtttaagaaacgtatgtagggtttcggttcgtttaaagcgtataaattttcgaggtagtagtttttaaatagagaggttagggagtgatcggagggtatttttttgtatatataggggttgttacggaattttcgtgaaatagattttcgggttagtcgcggcgtttaagttttttttgaggttttagaaaggtttattttgggttcgttcggggcggcggttttcggtaggtttagcgcggcggggcggggtcggcggtacgaggattaggtagcgcgggtagtacgcgttcgggtcggagttcgtttataagggcgcgcggcgcgttttgattggaaggtttcgagcgggggcggggtttttagggtgcgttttcgtcgtcgtttgttttcgtcgcggagattcggtagttgggggatgtcgacgtttgggttttgaggatgttgcggaagtttattatcgagtagattaacgattggtttattatcggtaagacggtgattaatgtggagttgttgggcgcgtcgttcgttttttcggtaggggcggttagggaggaggtgtagcgttaggacgtggttttcggcgttggtttcgcggtttaggtttcggttttggtttaagtttcggttcggtcggtcgttgcgttcgaaaggtaggacgcgggcggggcgg

SEQ ID NO: 137 - DNA, 24 bp, Artificial Sequence
  F1 Primer for Bisulfite converted genomic DNA from chr14104394180-104395081
ggtgtttaagaaacgtatgtaggg

SEQ ID NO: 138 - DNA, 20 bp, Artificial Sequence
  R1 Primer for Bisulfite converted genomic DNA from chr14104394180-104395081
aacttccgcaacatcctcaa

SEQ ID NO: 139 - DNA, 623 bp, Homo sapiens
tagccgggcgtggcggcgggcgcctgtagtcccagctgctcgggaggctgaggcaggagaagggcgtgaacccgggaggcggagcttgcagtgagccgagatcgcaccactgcgctccagcctgggggacagagcgagactcggtctcaagaaaaaaagaaaaagaaaaaaagaaaacggggtggacaaatcgatggggaggcggctcaacctttttccttctcaggcagaagcccctgagtaaggccgccgctgccacccactgcccctgaaaggacatctgagcacagtgattccggtggtgccaggactccaaccagcaaggagcgtttcgcacagagctgcagagagtctgtgccttccaacgagagccgtgtcccgaacgccatccacttagctctcactctcctccttcctcctttccctgccgctttctgtttttcatcagggccttggaactcgctagtacagaccgactttcaagttaagtctttcaaggaaaatgactcttggtttccagcctggatgtgtcattacaccctcatgcgtctcagggaaattagcaacctgattcttgccctgactgtcatttaaaataaaaagctttcttatttgccatctaa

SEQ ID NO: 140 - DNA, 623 bp, Artificial Sequence
  Bisulfite converted genomic DNA from chr15101389022-101389644
tagtcgggcgtggcggcgggcgtttgtagttttagttgttcgggaggttgaggtaggagaagggcgtgaattcgggaggcggagtttgtagtgagtcgagatcgtattattgcgttttagtttgggggatagagcgagattcggttttaagaaaaaaagaaaaagaaaaaaagaaaacggggtggataaatcgatggggaggcggtttaattttttttttttttaggtagaagtttttgagtaaggtcgtcgttgttatttattgtttttgaaaggatatttgagtatagtgatttcggtggtgttaggattttaattagtaaggagcgtttcgtatagagttgtagagagtttgtgttttttaacgagagtcgtgtttcgaacgttatttatttagtttttatttttttttttttttttttttttgtcgttttttgttttttattagggttttggaattcgttagtatagatcgatttttaagttaagttttttaaggaaaatgatttttggtttttagtttggatgtgttattatatttttatgcgttttagggaaattagtaatttgatttttgttttgattgttatttaaaataaaaagtttttttatttgttatttaa

SEQ ID NO: 141 - DNA, 20 bp, Artificial Sequence
  F1 Primer for Bisulfite converted genomic DNA from chr15101389022-101389644
ggaggcggagtttgtagtga

SEQ ID NO: 142 - DNA, 20 bp, Artificial Sequence
  R1 Primer for Bisulfite converted genomic DNA from chr15101389022-101389644
aacgaattccaaaaccctaa

SEQ ID NO: 143 - DNA, 699 bp, Homo sapiens
ggccgcagggaggcagccattttccgtaggcggtggccgcggcgcttccgggccgcgcggctggagcctgaggcggggccaccgtcgggcacgagaaaccattctctccccgcttttcgcagctgtatcccagaggcgcggctgaagctcgctgggacactgcccgcttgcccagtgcccacgggcacgcctgtcgcagacacaggctgtggcgaatccgagtgctggcgggcagcagtgcctgcagctgcgcccgcagactctttcccgcctccgccggcatttccgtgtccggccgatgtcatgcggtcggttgagcctcagggctagcagcttctactgatgcaaaaacataggaagtgtctctttgcttctggaaccggggtgggcagcaaggagctggccacttctttgaataccggtggcatcctgggcagaatttcggtgccgtttctgctggctcacttcaggaaaagtgaggctcacggctctcctcagccccccttagaagtctaaatgacctttggagaaatacaaagtcagcagaaagtttcactttcatcgtcaccaactcctgttggagttgatatttcataaacatcagcgacagaaagaaaatacgacttgatattttgtaaataaaatatccttttttacaagttgatattttgtaaacagaggtcagctgtaattttctcc

SEQ ID NO: 144 - DNA, 699 bp, Artificial Sequence
  Bisulfite converted genomic DNA from chr163355701-3356399
ggtcgtagggaggtagttatttttcgtaggcggtggtcgcggcgttttcgggtcgcgcggttggagtttgaggcggggttatcgtcgggtacgagaaattatttttttttcgtttttcgtagttgtattttagaggcgcggttgaagttcgttgggatattgttcgtttgtttagtgtttacgggtacgtttgtcgtagatataggttgtggcgaattcgagtgttggcgggtagtagtgtttgtagttgcgttcgtagattttttttcgttttcgtcggtattttcgtgttcggtcgatgttatgcggtcggttgagttttagggttagtagtttttattgatgtaaaaatataggaagtgtttttttgtttttggaatcggggtgggtagtaaggagttggttatttttttgaatatcggtggtattttgggtagaatttcggtgtcgtttttgttggtttattttaggaaaagtgaggtttacggttttttttagttttttttagaagtttaaatgatttttggagaaatataaagttagtagaaagttttatttttatcgttattaatttttgttggagttgatattttataaatattagcgatagaaagaaaatacgatttgatattttgtaaataaaatatttttttttataagttgatattttgtaaatagaggttagttgtaatttttttt

SEQ ID NO: 145 - DNA, 20 bp, Artificial Sequence
  F1 Primer for Bisulfite converted genomic DNA from chr163355701-3356399
gtattttagaggcgcggttg

SEQ ID NO: 146 - DNA, 23 bp, Artificial Sequence
  R1 Primer for Bisulfite converted genomic DNA from chr163355701-3356399
aaaaccgtaaacctcacttttcc

SEQ ID NO: 147 - DNA, 1204 bp, Homo sapiens
gacttttgtctccaaaagcatttgatccctggcagacacatacttatttccatacgggtcttgggggaagccaggaggagagagagctgattatcagaatcctgaaggtgaagctcaggtgggccccactgatgtcaaaggcaggcctgggcacacattcggaggccataatttgctgcctgggagggaggagcggcgggctgcagcggggtgtggacggggcattgttctcgggctctggtgcactgagcgtgacctgctccagccacaacggatagtccctgcctaccccctctcttagcaggaacccccctcgggaaagctgaccccagagtggcatcattaatgcacacacctgtctaggagaagaccaatgacttccgcctggtgttgactaggggcatgactagccacctacatccaggatgtggcaatgaaggacacaaaagctagagccgggaacacttgggtttgagccctagttccaccactcactcgctgtgtggactggagtaagttactttgcctctctgagcttctgcttctgccactagaaaataggcagagtcattacggggcttttgtatggcagccaagcagtcttatttggccacaggacatctgcccttgctgagacccccagaccatcccacactccttccctcactacactcaggggtctgcctaaaggacacctcctcagggaggccttactcgagacccccatctaacacagcacacacccccgtcctttccagcacctggcccacctttaattcctggatagcgcttactaccacctggtatattacagggttcattgtttaattcattctcattccctccgtccctccctccctccggcacacagacagagtaacataaactccatgagggctgctttgcccactgctatgtcaccagtgcctagaacaggatctggcaccaggagacgcttgacgagcaggaaatgaatgatttatgtaaagcacttagcacagagtttgcggtagggtgggagtgctggtaaatgccatcactatgcacccatggtcacccctaatctcacaggactcggcttgtaaaaacctcaactccagtaacgctttctggaaccccacggaacaacagcctgctccaagactgagggcgtggagaggctccctcagctgacagaagaggaagggacgcttagagaggcagg

SEQ ID NO: 148 - DNA, 1204 bp, Artificial Sequence
  Bisulfite converted genomic DNA from chr1649563509-49564712
gatttttgtttttaaaagtatttgatttttggtagatatatatttatttttatacgggttttgggggaagttaggaggagagagagttgattattagaattttgaaggtgaagtttaggtgggttttattgatgttaaaggtaggtttgggtatatattcggaggttataatttgttgtttgggagggaggagcggcgggttgtagcggggtgtggacggggtattgttttcgggttttggtgtattgagcgtgatttgttttagttataacggatagtttttgtttatttttttttttagtaggaatttttttcgggaaagttgattttagagtggtattattaatgtatatatttgtttaggagaagattaatgattttcgtttggtgttgattaggggtatgattagttatttatatttaggatgtggtaatgaaggatataaaagttagagtcgggaatatttgggtttgagttttagttttattatttattcgttgtgtggattggagtaagttattttgtttttttgagtttttgtttttgttattagaaaataggtagagttattacggggtttttgtatggtagttaagtagttttatttggttataggatatttgtttttgttgagatttttagattattttatatttttttttttattatatttaggggtttgtttaaaggatatttttttagggaggttttattcgagatttttatttaatatagtatatattttcgttttttttagtatttggtttatttttaatttttggatagcgtttattattatttggtatattatagggtttattgtttaatttatttttatttttttcgttttttttttttttcggtatatagatagagtaatataaattttatgagggttgttttgtttattgttatgttattagtgtttagaataggatttggtattaggagacgtttgacgagtaggaaatgaatgatttatgtaaagtatttagtatagagtttgcggtagggtgggagtgttggtaaatgttattattatgtatttatggttatttttaattttataggattcggtttgtaaaaattttaattttagtaacgttttttggaattttacggaataatagtttgttttaagattgagggcgtggagaggtttttttagttgatagaagaggaagggacgtttagagaggtagg

SEQ ID NO: 149 - DNA, 20 bp, Artificial Sequence
  F1 Primer for Bisulfite converted genomic DNA from chr1649563509-49564712
ttgttttcgggttttggtgt

SEQ ID NO: 150 - DNA, 20 bp, Artificial Sequence
  R1 Primer for Bisulfite converted genomic DNA from chr1649563509-49564712
ccaccctaccgcaaactcta

SEQ ID NO: 151 - DNA, 1074 bp, Homo sapiens
tgacatagtccgccagagtgtggccgaggagtaggaggagtgtgaagaggctggggtactgtccagaccagagacggtggtggccttgacatctccccagagatggggatggaggggagggaccatattccagagctgtttgggaggcagaagggagactgaggggagggtggggcatgaaagacagggaggtgcccagggcaggcaggttcctgccttgacagcaccaacaggagcaggaatctctccacggactgaggcgccgggcagggaggggatggttcctgagaggccaacctgcctcccagtcctgggccccggggctggcggaggcctcctgtacacgcacacgcacgcacatgcgcacgcacacgcacgcgcacagacgctgcctggattttgctttgggttccgtcttctcactgcggaccctggattgaaacgatctccccgcggccgccgccgctacctggtgcccgcaggtgcctgcaggagtcctggggccagctggcctcgatgtacgtcagcacgcgggaacggtacaagtggctgcgcttcagcgaggactgtctgtacctgaacgtgtacgcgccggcgcgcgcgcccggggatccccagctgccagtgagtgccaggtctcccgcgcccgcggtcccaccgccgcccaccgccccgctcagatcccggccttcttcgtccaggtgatggtctggttcccgggaggcgccttcatcgtgggcgctgcttcttcgtacgagggctctgacttggccgcccgcgagaaagtggtgctggtgtttctgcagcacaggctcggcatcttcggcttcctgaggtggcggggccggtaccctttgggaccgcagctgtggccagagcggcggggactgggtgggaagggaggggcggggcctggggcggggatggggggggtggggccgcgaggcgggggcggggcctggcgctcggtggaaggggcgggcgctccataccatctggatggggcgagctaactccaaggaagggggtgtggtcgcaggactgggtcttagaggggcaaggctgggctgggtg

SEQ ID NO: 152 - DNA, 1074 bp, Artificial Sequence
  Bisulfite converted genomic DNA from chr1667034059-67035132
tgatatagttcgttagagtgtggtcgaggagtaggaggagtgtgaagaggttggggtattgtttagattagagacggtggtggttttgatatttttttagagatggggatggaggggagggattatattttagagttgtttgggaggtagaagggagattgaggggagggtggggtatgaaagatagggaggtgtttagggtaggtaggtttttgttttgatagtattaataggagtaggaatttttttacggattgaggcgtcgggtagggaggggatggtttttgagaggttaatttgttttttagttttgggtttcggggttggcggaggttttttgtatacgtatacgtacgtatatgcgtacgtatacgtacgcgtatagacgttgtttggattttgttttgggtttcgtttttttattgcggattttggattgaaacgattttttcgcggtcgtcgtcgttatttggtgttcgtaggtgtttgtaggagttttggggttagttggtttcgatgtacgttagtacgcgggaacggtataagtggttgcgttttagcgaggattgtttgtatttgaacgtgtacgcgtcggcgcgcgcgttcggggatttttagttgttagtgagtgttaggtttttcgcgttcgcggttttatcgtcgtttatcgtttcgtttagatttcggtttttttcgtttaggtgatggtttggttttcgggaggcgtttttatcgtgggcgttgttttttcgtacgagggttttgatttggtcgttcgcgagaaagtggtgttggtgtttttgtagtataggttcggtattttcggttttttgaggtggcggggtcggtattttttgggatcgtagttgtggttagagcggcggggattgggtgggaagggaggggcggggtttggggcggggatggggggggtggggtcgcgaggcgggggcggggtttggcgttcggtggaaggggcgggcgttttatattatttggatggggcgagttaattttaaggaagggggtgtggtcgtaggattgggttttagaggggtaaggttgggttgggtg

SEQ ID NO: 153 - DNA, 20 bp, Artificial Sequence
  F1 Primer for Bisulfite converted genomic DNA from chr1667034059-67035132
agggaggtgtttagggtagg

SEQ ID NO: 154 - DNA, 21 bp, Artificial Sequence
  R1 Primer for Bisulfite converted genomic DNA from chr1667034059-67035132
ccacaactacgatcccaaaaa

SEQ ID NO: 155 - DNA, 768 bp, Homo sapiens
tttcggctcgctgccatccactttcacctcggccgcctgcacggccgccatcttcccggagggcccgacccaaaagtaaggaggatagtacgttaatttccaggtcaaggtgcgaagccccacctcttccgggggagagggggcgggcactctcgatgcgccgcggctgctgctgcgcaggcccagtgctgcgcttcgcggcagaggcgtctgcggtgacagctcagtcagttgagctctgtgtgccaggcgctcgcgagggggtagctcttctagtagtgctcggcgtcagacatggcggaggcgatggatttgggcaaagaccccaacgggcccacccattcctcgactctgttcgtgagggacgacggcagctccatgtccttctacgtgcggcccagcccggccaagcgtcggctgtcgacgctcatcctgcacggcggcggcaccgtgtgccgagtgcaggagcccggggccgtgctgctggcccagcccggggaggcgctggccgaggcctcgggtgatttcatctccacgcagtacatcctggactgcgtggagcgcaacgagaggctggagctggaggcctatcggctgggccccgcctcggcggcggacaccggctcggaagcaaagcccggggccctggccgagggcgccgcggagccggagccgcagcggcacgccgggcggatcgccttcacggatgcggacgacgtagccatccttacctacgtgaaggaaaatgcccgctcgcccagctccgtc

SEQ ID NO: 156 - DNA, 768 bp, Artificial Sequence
  Bisulfite converted genomic DNA from chr1675681487-75682254
tttcggttcgttgttatttatttttatttcggtcgtttgtacggtcgttattttttcggagggttcgatttaaaagtaaggaggatagtacgttaatttttaggttaaggtgcgaagttttatttttttcgggggagagggggcgggtattttcgatgcgtcgcggttgttgttgcgtaggtttagtgttgcgtttcgcggtagaggcgtttgcggtgatagtttagttagttgagttttgtgtgttaggcgttcgcgagggggtagtttttttagtagtgttcggcgttagatatggcggaggcgatggatttgggtaaagattttaacgggtttatttatttttcgattttgttcgtgagggacgacggtagttttatgtttttttacgtgcggtttagttcggttaagcgtcggttgtcgacgtttattttgtacggcggcggtatcgtgtgtcgagtgtaggagttcggggtcgtgttgttggtttagttcggggaggcgttggtcgaggtttcgggtgattttatttttacgtagtatattttggattgcgtggagcgtaacgagaggttggagttggaggtttatcggttgggtttcgtttcggcggcggatatcggttcggaagtaaagttcggggttttggtcgagggcgtcgcggagtcggagtcgtagcggtacgtcgggcggatcgtttttacggatgcggacgacgtagttatttttatttacgtgaaggaaaatgttcgttcgtttagtttcgtt

SEQ ID NO: 157 - DNA, 21 bp, Artificial Sequence
  F1 Primer for Bisulfite converted genomic DNA from chr1675681487-75682254
gcgtttgcggtgatagtttag

SEQ ID NO: 158 - DNA, 20 bp, Artificial Sequence
  R1 Primer for Bisulfite converted genomic DNA from chr1675681487-75682254
ccgataaacctccaactcca

SEQ ID NO: 159 - DNA, 1251 bp, Homo sapiens
agacccaagtatggaataagaacccgcaccccgacccctgccctgcgcagtgaggattcccgcgtgcagccgaagccccagcaagacacttccaaaccagcgctagcaggggagatgaactttccacgttgctgatggaaaagccacccccagctcttcccgccaacccaggtctctggtgcaggacagaggagcgtcacagctgggccactggcccggcccccagcccaaccgagtgtggggacacctccggtggagaatgaagaaccaaagtacctcaaagcttggcgtgtctgccagagctggagcagcagccagagggacgcctctccccctgggctcagcaaaagctcccagacgtcgtctcttccaagttccaccgcacagccagctctttctcttcactgggaggttccgcaggggctgacttgctccttaaggtcccaagactattaagggacacccagtgaggctgggaaaggggctctaagaggcatggacgaagagtcgggggtaaccaagccactggaagctcctggagactcgaggcagagcgaatctgggaacagggcaggagtcccagaatgggatgttgaggatgggggtgtcctggggctaccacagggaaggacccggcgcagagctccctccggagcccggcactgcggcccgcgccctctactccccgcgcgccggccaacaaccctcggcgctgccccgcaatcttcagcgctgcctggtgaggagctttcagacgtggctgcggtcccagggtggccggcgggagccagggaaatgggagaaggaaggaaggagcaggttttgcccccagctccctctccccacccccgaccgaggggccatgtctcggggaggggcgctggccgagggcagagcaggagggcccaggtgggctggggctctaagaggcgcaccttttagccggggatgtcaaaggacgctggcagagtcacttctcatctaactgaccacaaatgaacgttgtaacccttcctcacccccccaatcgccagagaacctgggagctcctgggaggacaaaggccctagtgaaggaagtcgaatcttaatcttggccctcctcgctcttgggggcatccttgcagccaagggacttggaatccaaacccttcccaaggccccaacctcagccctaagccaagacccaggcaccttgatgacacaggaatccgtgctgcctcccggtgcagtgtcccagctgggaggagtt

SEQ ID NO: 160 - DNA, 1251 bp, Artificial Sequence
  Bisulfite converted genomic DNA from chr1675568749-75569999
agatttaagtatggaataagaattcgtatttcgatttttgttttgcgtagtgaggattttcgcgtgtagtcgaagttttagtaagatatttttaaattagcgttagtaggggagatgaattttttacgttgttgatggaaaagttatttttagtttttttcgttaatttaggtttttggtgtaggatagaggagcgttatagttgggttattggttcggtttttagtttaatcgagtgtggggatattttcggtggagaatgaagaattaaagtattttaaagtttggcgtgtttgttagagttggagtagtagttagagggacgtttttttttttgggtttagtaaaagtttttagacgtcgttttttttaagttttatcgtatagttagttttttttttttattgggaggtttcgtaggggttgatttgttttttaaggttttaagattattaagggatatttagtgaggttgggaaaggggttttaagaggtatggacgaagagtcgggggtaattaagttattggaagtttttggagattcgaggtagagcgaatttgggaatagggtaggagttttagaatgggatgttgaggatgggggtgttttggggttattatagggaaggattcggcgtagagtttttttcggagttcggtattgcggttcgcgttttttatttttcgcgcgtcggttaataattttcggcgttgtttcgtaatttttagcgttgtttggtgaggagtttttagacgtggttgcggttttagggtggtcggcgggagttagggaaatgggagaaggaaggaaggagtaggttttgtttttagttttttttttttattttcgatcgaggggttatgtttcggggaggggcgttggtcgagggtagagtaggagggtttaggtgggttggggttttaagaggcgtattttttagtcggggatgttaaaggacgttggtagagttattttttatttaattgattataaatgaacgttgtaatttttttttatttttttaatcgttagagaatttgggagtttttgggaggataaaggttttagtgaaggaagtcgaattttaattttggtttttttcgtttttgggggtatttttgtagttaagggatttggaatttaaattttttttaaggttttaattttagttttaagttaagatttaggtattttgatgatataggaattcgtgttgtttttcggtgtagtgttttagttgggaggagtt

SEQ ID NO: 161 - DNA, 20 bp, Artificial Sequence
  F1 Primer for Bisulfite converted genomic DNA from chr1675568749-75569999
ttgggttattggttcggttt

SEQ ID NO: 162 - DNA, 20 bp, Artificial Sequence
  R1 Primer for Bisulfite converted genomic DNA from chr1675568749-75569999
aaccgcaaccacgtctaaaa

SEQ ID NO: 163 - DNA, 20 bp, Artificial Sequence
  F2 Primer for Bisulfite converted genomic DNA from chr1675568749-75569999
tttcggcgttgtttcgtaat

SEQ ID NO: 164 - DNA, 20 bp, Artificial Sequence
  R2 Primer for Bisulfite converted genomic DNA from chr1675568749-75569999
tcccaaaaactcccaaattc

SEQ ID NO: 165 - DNA, 806 bp, Homo sapiens
tgccaccggtgtggggaaaggtgagcgcggactgcggtgagagctggggccctgggactcagaggtgtcactgggggccggggccattcggagggtgctgctgccccacccatgagaaacctgggaagcggggtaggggctgaggtcgggtgcacttggtgcgcctgataggaaaagcgagaggaacaggagagatgagaggcattttcctgcccgagcggaaggacagagacgcccgctggggtgggcacggcaggcccggggggaggctggcgccccgcagtaccagctgctcctcctcacctccacctctccccggctgtgctgtggaggggaaacggagccaggattctctgaaggttcaccgaccccgccgtcctccctgaagaccccaaaacctgagctgtgtactgtcgcgcaggaggcagagtttgagatcgacgtccccctgcacctggggcgcctcctgatggtgaagctgcgcaaacacaacgtgctgttgagtctcgactggttctgcaagtggatctcagtgcagggcccggggacccaaggcgaggcctttttcccctgctaccgctgggtgcagggccacggaattatctgcctgcctgagggtactggtgagctcggtggagaaggggcacagcccctggcaggctggaggaagaagcgggtggggggaggctactgtaaaagagggaaaatgagagatgagggagatggggagaagtgaagggggagggtggctgatggggagacccgagcgcggaggggtgaccagggagggcatgtgctcttgtccc

SEQ ID NO: 166 - DNA, 806 bp, Artificial Sequence
  Bisulfite converted genomic DNA from chr176797216-6798021
tgttatcggtgtggggaaaggtgagcgcggattgcggtgagagttggggttttgggatttagaggtgttattgggggtcggggttattcggagggtgttgttgttttatttatgagaaatttgggaagcggggtaggggttgaggtcgggtgtatttggtgcgtttgataggaaaagcgagaggaataggagagatgagaggtatttttttgttcgagcggaaggatagagacgttcgttggggtgggtacggtaggttcggggggaggttggcgtttcgtagtattagttgtttttttttatttttattttttttcggttgtgttgtggaggggaaacggagttaggattttttgaaggtttatcgatttcgtcgttttttttgaagattttaaaatttgagttgtgtattgtcgcgtaggaggtagagtttgagatcgacgtttttttgtatttggggcgttttttgatggtgaagttgcgtaaatataacgtgttgttgagtttcgattggttttgtaagtggattttagtgtagggttcggggatttaaggcgaggttttttttttttgttatcgttgggtgtagggttacggaattatttgtttgtttgagggtattggtgagttcggtggagaaggggtatagtttttggtaggttggaggaagaagcgggtggggggaggttattgtaaaagagggaaaatgagagatgagggagatggggagaagtgaagggggagggtggttgatggggagattcgagcgcggaggggtgattagggagggtatgtgtttttgtttt

SEQ ID NO: 167 - DNA, 20 bp, Artificial Sequence
  F1 Primer for Bisulfite converted genomic DNA from chr176797216-6798021
gttcgagcggaaggatagag

SEQ ID NO: 168 - DNA, 20 bp, Artificial Sequence
  R1 Primer for Bisulfite converted genomic DNA from chr176797216-6798021
tccgtaaccctacacccaac

SEQ ID NO: 169 - DNA, 1155 bp, Homo sapiens
caggaccacgggccccgcgccaccatgccccggcagctccctcggcactcgcgggaccccaaggcgggctgctgcggcttccccaaacccaccgccaccccaccctgtctgaccacagcccccccaaaccgcctgccgcctgccccttagagcacaacgcagcctggccctacacctaacccccagaccactccctcaatcgtgaacccagtaaaccagcctgcaccccacacagcccaaggtcttgccccgcgcccaggtcccagcacctagcgacccgccttcagcacttggaatgggctggagttgggaaatcagagagggccagtgcaggtgctggggcagtatcgagagcggggaccttactgctagggtggagaggagtgggggagattggtcccaacccatcaccgctggtggatgtctcagaggaagcctggggtccctggccactgggtaggagggtaggaagaaaagatgaggcaggctcttcccgcccaaacaagtccagggggcggaggacgggggagtgtctacagggtgtggggagggggctgccccgcctcacgattaaggttcagactaggggtacttaaccgctggagcaggcccgactgaggtggggtgagctgaggaccccgaggaggagggcacaaggctgccgggggtgatgggggcaggggctgcttcacgtggtggctgctgactggccaagacctagctttgccccaggcttaggtcagcgacccgactgcaggaaagggctcaggctttggtcccaggcacagggcctgggccaaaaaaaaaaaaaaagccttttttctttttctctcatttttttttctttagtagtaggcacagtgggcacaggttacaaggtgctgctttacatgcgtattcgcctcgcaggtgtgaccttggaccgttgccctcctctctctggaccccaaatacattgcatatgtaggtactaaatccatatttgttaaatggcttgatgtggataacataggagattatccctggctggtctccattaggccctctaagcacacccagcagctgtatacactcccacccagcttgtcagagaagatgggaccaggggagctgtcgctctggtggagtgcagggggtcaaagcaagcctcaaggg

SEQ ID NO: 170 - DNA, 1155 bp, Artificial Sequence
  Bisulfite converted genomic DNA from chr178066419-8067573
taggattacgggtttcgcgttattatgtttcggtagttttttcggtattcgcgggattttaaggcgggttgttgcggtttttttaaatttatcgttattttattttgtttgattatagtttttttaaatcgtttgtcgtttgttttttagagtataacgtagtttggttttatatttaatttttagattatttttttaatcgtgaatttagtaaattagtttgtattttatatagtttaaggttttgtttcgcgtttaggttttagtatttagcgattcgtttttagtatttggaatgggttggagttgggaaattagagagggttagtgtaggtgttggggtagtatcgagagcggggattttattgttagggtggagaggagtgggggagattggttttaatttattatcgttggtggatgttttagaggaagtttggggtttttggttattgggtaggagggtaggaagaaaagatgaggtaggtttttttcgtttaaataagtttagggggcggaggacgggggagtgtttatagggtgtggggagggggttgtttcgttttacgattaaggtttagattaggggtatttaatcgttggagtaggttcgattgaggtggggtgagttgaggatttcgaggaggagggtataaggttgtcgggggtgatgggggtaggggttgttttacgtggtggttgttgattggttaagatttagttttgttttaggtttaggttagcgattcgattgtaggaaagggtttaggttttggttttaggtatagggtttgggttaaaaaaaaaaaaaaagtttttttttttttttttttatttttttttttttagtagtaggtatagtgggtataggttataaggtgttgttttatatgcgtattcgtttcgtaggtgtgattttggatcgttgtttttttttttttggattttaaatatattgtatatgtaggtattaaatttatatttgttaaatggtttgatgtggataatataggagattatttttggttggtttttattaggttttttaagtatatttagtagttgtatatatttttatttagtttgttagagaagatgggattaggggagttgtcgttttggtggagtgtagggggttaaagtaagttttaaggg

SEQ ID NO: 171 - DNA, 27 bp, Artificial Sequence
  F1 Primer for Bisulfite converted genomic DNA from chr178066419-8067573
cgtagtttggttttatatttaattttt

SEQ ID NO: 172 - DNA, 20 bp, Artificial Sequence
  R1 Primer for Bisulfite converted genomic DNA from chr178066419-8067573
taccctcctcctcgaaatcc

SEQ ID NO: 173 - DNA, 20 bp, Artificial Sequence
  F2 Primer for Bisulfite converted genomic DNA from chr178066419-8067573
gagggggttgtttcgtttta

SEQ ID NO: 174 - DNA, 21 bp, Artificial Sequence
  R2 Primer for Bisulfite converted genomic DNA from chr178066419-8067573
caacgatccaaaatcacacct

SEQ ID NO: 175 - DNA, 1443 bp, Homo sapiens
ctgggattacaggcgcccaccatcatgcctggctaatttttgtatttttagtagagacggggtttcaccgtattggcaaggtttgtctcgaactcctgacctcaggtgatccatccgcattggcctcccaaaatgctgggattacaggtgtgagccaccgcacccggcctctggggacccatttcaacagtctggcacccatggttgggaatatgtttaacctctaaattggggttttcctgctgaactgcgtctgaaagactgcttctctgagctagctcatgtttttgctttttctctctattgattcctttatagaatattgctgtcatataagtccagcttttccgtgaatttaacagttttatcctgttatcgatgtgtttttattttcttgaaaataagcagattgagacatcagatgggagaaaggcttgactatgtcgccagcagcctcatgccaaattcctgattcctggaggacagagcccccctcctgctccaaggccaggagatggcgccactggacagtgagtctgggattcctagcacacacttgtgattgctggtccaggaaaaagaagcaggaagggccattagtacaatcagtcaggaaattctgcttaagtgcacatttctccccaggagagtcgggtcactgcactgggaggaagcccgcctgccaagggctgtgtctggaacctgttcatcgcgctccactcagcacagtgagatcgcctcttctgttactcggtctccagggggcgggacctggagccgtatccaatcagggccatggggtggggtcgtgagaactgtggatcaggcgcactgcagggaggtgggtgcgcggcatcttcacccctctaaatgcggctcctactctgtagctgacagacacccacagacgtgtctgtagcatcctctgtcacactgggacccgtactggcagcaggagccgtagggaggactgggacacctggaagccaggaattggtgagtgtgcggggctgagggtcccgagactaggatgggactggttggaaccggcaggaatagaccgtggcgggacccgggcctcttcgtagttgactctgggttctgagacccaagtccccctggcgcacctcggccctggttcctctcaggccgcagggtggggctgcgacggcagccgagacccctagccgtccggtccagtccctgtgcaactgactgtggccccaggcccagcgccctctctggtcagctccgcgcccgctgccccagtcacgcccgctgcctccgtgtctccccagattgtacgggggccactagagggtcatgggggaaatcctgcctcgagtatggggttcgtgtgagaggagctgtaggctgtggggtccccagttccctctttctcaccaggggcttttgtattttatctgagtttcccaaatatac

SEQ ID NO: 176 - DNA, 1443 bp, Artificial Sequence
  Bisulfite converted genomic DNA from chr1911783996-11785438
ttgggattataggcgtttattattatgtttggttaatttttgtatttttagtagagacggggttttatcgtattggtaaggtttgtttcgaatttttgattttaggtgatttattcgtattggttttttaaaatgttgggattataggtgtgagttatcgtattcggtttttggggatttattttaatagtttggtatttatggttgggaatatgtttaatttttaaattggggtttttttgttgaattgcgtttgaaagattgtttttttgagttagtttatgtttttgttttttttttttattgatttttttatagaatattgttgttatataagtttagttttttcgtgaatttaatagttttattttgttatcgatgtgtttttatttttttgaaaataagtagattgagatattagatgggagaaaggtttgattatgtcgttagtagttttatgttaaatttttgatttttggaggatagagttttttttttgttttaaggttaggagatggcgttattggatagtgagtttgggatttttagtatatatttgtgattgttggtttaggaaaaagaagtaggaagggttattagtataattagttaggaaattttgtttaagtgtatatttttttttaggagagtcgggttattgtattgggaggaagttcgtttgttaagggttgtgtttggaatttgtttatcgcgttttatttagtatagtgagatcgttttttttgttattcggtttttagggggcgggatttggagtcgtatttaattagggttatggggtggggtcgtgagaattgtggattaggcgtattgtagggaggtgggtgcgcggtatttttatttttttaaatgcggtttttattttgtagttgatagatatttatagacgtgtttgtagtattttttgttatattgggattcgtattggtagtaggagtcgtagggaggattgggatatttggaagttaggaattggtgagtgtgcggggttgagggtttcgagattaggatgggattggttggaatcggtaggaatagatcgtggcgggattcgggttttttcgtagttgattttgggttttgagatttaagtttttttggcgtatttcggttttggttttttttaggtcgtagggtggggttgcgacggtagtcgagatttttagtcgttcggtttagtttttgtgtaattgattgtggttttaggtttagcgttttttttggttagtttcgcgttcgttgttttagttacgttcgttgttttcgtgtttttttagattgtacgggggttattagagggttatgggggaaattttgtttcgagtatggggttcgtgtgagaggagttgtaggttgtggggtttttagtttttttttttttattaggggtttttgtattttatttgagttttttaaatatat

SEQ ID NO: 177 - DNA, 23 bp, Artificial Sequence
  F1 Primer for Bisulfite converted genomic DNA from chr1911783996-11785438
tttggtatttatggttgggaata

SEQ ID NO: 178 - DNA, 18 bp, Artificial Sequence
  R1 Primer for Bisulfite converted genomic DNA from chr1911783996-11785438
aaatcccgccccctaaaa

SEQ ID NO: 179 - DNA, 20 bp, Artificial Sequence
  F2 Primer for Bisulfite converted genomic DNA from chr1911783996-11785438
ttgggaggaagttcgtttgt

SEQ ID NO: 180 - DNA, 20 bp, Artificial Sequence
  R2 Primer for Bisulfite converted genomic DNA from chr1911783996-11785438
caacgaacgcgaaactaacc

SEQ ID NO: 181 - DNA, 1480 bp, Homo sapiens
aatacgaagttgacaagatgttattaaagacgaataaaattataccaatacctagacacctgatcccaacattaaacatgaaatacactaaaccagagttattttctggaggagtatttaaaatgatggggatgttattacttgggtcgcaatgaacctgaaaacccattttctacagaatatacagcagcagggaagcaaggggaccagcagacccctttttaagtacgcatgtgataagcaatgaacacgaactgcccagagcagtctccaacactgacacgattcgcttccccaccacgacgccctagcgctactgtgcaacgaagacctcccaagcactggttccaatgcggagaccatgggctcccagactctgggaactccaacacgactgcgaaacgaactccgagcgaggactccccgagagctccccgcaacacggacctcacgcgctagcgaacaacagaaaaaaaaaagcgcgctctccctgcccctgaaacattcccagaagcccacgcagaccagaccgatgacctgtctccactgctggaggcgagtcagggacccgaagtctctaaacactcgcctctacccgccgccccgcgaaccccacacactgcagacgcgacactcgcaagtttcggggatggcggccggcgagggccatactgcgtctttccggagacacggaatacggcaccagccgtccctttatgatgcaatatgtctgcgcccaggggacgcttgctgggagcagccattttcaaccctactgccgtagagcaggcggagtccctcttttcgcgccttaagacaggtaggttctgacgatgaaaagcaattgaaaacgacccatttcaccctttttccagtccacgtgaactgctagatcttggctttgcaacattagccaggggcgctacataaactgcttagtttctcaaaggctcaagcctgccctgatctgtctacaggatgggtagagatggtcacagacatttaggcactttgatcctaagaagaatggaaagaaaccatgtggcgcggcagtcttacaggaatttcaagagggagggacctgagcaacaatcggaggggttattactcctgaggatgcatctggctggagaaagcagcctttgagaactgcctaagaagtatctttacatctacatcagatgtagcctcagaggaaggagtcagtcatagaatggataaaacaaccgcgtgttaaagctttggttataattggtgtggagaatggagaacacagttgatcagcagttgacaaagtggggaaccaacacgaaaacagggctctctcaccctggaaaagccaaaggcagaacaagcctttacatccaggaaggtgggagcaacttgaaatcaaaactctgaaggggagatgattctgttcaatactgaaactcagctgatcaattaactgatgaattctagcaccaaacctgctccctat

SEQ ID NO: 182 - DNA, 1480 bp, Artificial Sequence
  Bisulfite converted genomic DNA from chr1952390560-52392039
aatacgaagttgataagatgttattaaagacgaataaaattatattaatatttagatatttgattttaatattaaatatgaaatatattaaattagagttattttttggaggagtatttaaaatgatggggatgttattatttgggtcgtaatgaatttgaaaatttattttttatagaatatatagtagtagggaagtaaggggattagtagatttttttttaagtacgtatgtgataagtaatgaatacgaattgtttagagtagtttttaatattgatacgattcgtttttttattacgacgttttagcgttattgtgtaacgaagattttttaagtattggttttaatgcggagattatgggtttttagattttgggaattttaatacgattgcgaaacgaatttcgagcgaggatttttcgagagtttttcgtaatacggattttacgcgttagcgaataatagaaaaaaaaaagcgcgttttttttgtttttgaaatatttttagaagtttacgtagattagatcgatgatttgtttttattgttggaggcgagttagggattcgaagtttttaaatattcgtttttattcgtcgtttcgcgaattttatatattgtagacgcgatattcgtaagtttcggggatggcggtcggcgagggttatattgcgttttttcggagatacggaatacggtattagtcgtttttttatgatgtaatatgtttgcgtttaggggacgtttgttgggagtagttatttttaattttattgtcgtagagtaggcggagttttttttttcgcgttttaagataggtaggttttgacgatgaaaagtaattgaaaacgatttattttattttttttttagtttacgtgaattgttagattttggttttgtaatattagttaggggcgttatataaattgtttagttttttaaaggtttaagtttgttttgatttgtttataggatgggtagagatggttatagatatttaggtattttgattttaagaagaatggaaagaaattatgtggcgcggtagttttataggaattttaagagggagggatttgagtaataatcggaggggttattatttttgaggatgtatttggttggagaaagtagtttttgagaattgtttaagaagtatttttatatttatattagatgtagttttagaggaaggagttagttatagaatggataaaataatcgcgtgttaaagttttggttataattggtgtggagaatggagaatatagttgattagtagttgataaagtggggaattaatacgaaaatagggtttttttattttggaaaagttaaaggtagaataagtttttatatttaggaaggtgggagtaatttgaaattaaaattttgaaggggagatgattttgtttaatattgaaatttagttgattaattaattgatgaattttagtattaaatttgttttttat

SEQ ID NO: 183 - DNA, 23 bp, Artificial Sequence
  F1 Primer for Bisulfite converted genomic DNA from chr1952390560-52392039
agggaagtaaggggattagtaga

SEQ ID NO: 184 - DNA, 20 bp, Artificial Sequence
  R1 Primer for Bisulfite converted genomic DNA from chr1952390560-52392039
ccaacaaacgtcccctaaac

SEQ ID NO: 185 - DNA, 20 bp, Artificial Sequence
  F2 Primer for Bisulfite converted genomic DNA from chr1952390560-52392039
ttcggagatacggaatacgg

SEQ ID NO: 186 - DNA, 20 bp, Artificial Sequence
  R2 Primer for Bisulfite converted genomic DNA from chr1952390560-52392039
ttctccattctccacaccaa

SEQ ID NO: 187 - DNA, 1364 bp, Homo sapiens
gggagggagggcttgtcttgctccggcccctgtgatgagtcccctcttacccggcaggtggtgactccccatccgcggaggctcccccgctgaccccaagtccccaccgctctgtccttctccaaggcccggtgcaggagcaccctttgtccctgggtggcctgaccctgctgaccacgggtcacctccctgaccacgaatactctgccaactgcctcctgagtgaccgagacagctcagtctagagtctcgactacccggggtgacccaatagtcccgactccccgagacagggacccggagacgtgggctttgtcagtttaacagattctgcctccgggaaccaactgccgggagattctgattggctcagccgcgtgcccacgtgaccgaccaaaaccggccaccagggtctcccaactgcctgtcaaacgcacagccctgttgggcccccggagaggaagagccgagaaggaggccaggccgggggtcaggtggcttcctctatgggccctttaatccacctcacttttttatcttcttttttttcccagcagctttattaagttataattcacacacgatgtaattcgcccatttaaaatgtacaatgggcccggcgcggtggctcacgcctgtaatcccagcactttgggaggccgagggggcagcggggatgggggtggcgggtcacctgaggtcatgagttcaagaccagccttgccaacatggcaaaaccctgtctctactaaagatacaaaaaatatccaggcatagttgtgtgtgcttgtaatcccaggtactcgggaggctgaggcaggagaatcgcttgaacctgggagatagaagttgctgtgacctgaaatcgcaccacttcactccagcctgggtgacagagtgagaccctgtctctaaaataaaatgaaataaaatagaatatacactggttcttagtatagtcacagaattgtgcaatcatcggcaccatctaatttgagaacattttctatatatatgtagagaaaggggtcttgctatgttgtccaggctggtcttgaactcttggggtgaaaccatcctccttcttcagcctgtgcaatacctatgcttgcacgggtgcaagcatcagcattacaggcatgagatgcctcacctggcccagaacttagttttttactgaaaaatattcccttgtatgcatatatgatatttatccagtcatcagctgatggacttttgaactgtttccacttctgtttattatgaataaagctgctatgagcatttgtgtagaagtttgtgtagatttatgttttcatttctgcttatataagtaggagtataatttctgggtcaaagtataa

SEQ ID NO: 188 - DNA, 1364 bp, Artificial Sequence
  Bisulfite converted genomic DNA from chr205484894-5486257
gggagggagggtttgttttgtttcggtttttgtgatgagtttttttttattcggtaggtggtgattttttattcgcggaggttttttcgttgattttaagtttttatcgttttgtttttttttaaggttcggtgtaggagtattttttgtttttgggtggtttgattttgttgattacgggttatttttttgattacgaatattttgttaattgttttttgagtgatcgagatagtttagtttagagtttcgattattcggggtgatttaatagtttcgatttttcgagatagggattcggagacgtgggttttgttagtttaatagattttgttttcgggaattaattgtcgggagattttgattggtttagtcgcgtgtttacgtgatcgattaaaatcggttattagggttttttaattgtttgttaaacgtatagttttgttgggttttcggagaggaagagtcgagaaggaggttaggtcgggggttaggtggttttttttatgggttttttaatttattttatttttttattttttttttttttttagtagttttattaagttataatttatatacgatgtaattcgtttatttaaaatgtataatgggttcggcgcggtggtttacgtttgtaattttagtattttgggaggtcgagggggtagcggggatgggggtggcgggttatttgaggttatgagtttaagattagttttgttaatatggtaaaattttgtttttattaaagatataaaaaatatttaggtatagttgtgtgtgtttgtaattttaggtattcgggaggttgaggtaggagaatcgtttgaatttgggagatagaagttgttgtgatttgaaatcgtattattttattttagtttgggtgatagagtgagattttgtttttaaaataaaatgaaataaaatagaatatatattggtttttagtatagttatagaattgtgtaattatcggtattatttaatttgagaatattttttatatatatgtagagaaaggggttttgttatgttgtttaggttggttttgaatttttggggtgaaattattttttttttttagtttgtgtaatatttatgtttgtacgggtgtaagtattagtattataggtatgagatgttttatttggtttagaatttagttttttattgaaaaatatttttttgtatgtatatatgatatttatttagttattagttgatggatttttgaattgtttttatttttgtttattatgaataaagttgttatgagtatttgtgtagaagtttgtgtagatttatgtttttatttttgtttatataagtaggagtataatttttgggttaaagtataa

SEQ ID NO: 189 - DNA, 24 bp, Artificial Sequence
  F1 Primer for Bisulfite converted genomic DNA from chr205484894-5486257
tttgattttgttgattacgggtta

SEQ ID NO: 190 - DNA, 23 bp, Artificial Sequence
  R1 Primer for Bisulfite converted genomic DNA from chr205484894-5486257
ttctatctcccaaattcaaacga

SEQ ID NO: 191 - DNA, 20 bp, Artificial Sequence
  F2 Primer for Bisulfite converted genomic DNA from chr205484894-5486257
attcgggaggttgaggtagg

SEQ ID NO: 192 - DNA, 24 bp, Artificial Sequence
  R2 Primer for Bisulfite converted genomic DNA from chr205484894-5486257
ttctaaaccaaataaaacatctca

SEQ ID NO: 193 - DNA, 1063 bp, Homo sapiens
ccagaacctggcctgtgatgggtcctcaggacaccttttgtccaggaacccaggggccaatgacaaccttcacaggcatctgctgagaagacatgggccagcgctggatttgtccagctctcccaggcttggtccaagggctcaggagtggacccatccctgccccgaaagatccattttaaaaatcagtgaccctgtacttttactctctttgcacttggttaagactttatccctgattttatttgatcggtaaggcagggactgccatcattccccattttatagacagaggacagacggccccatggctccgtgtcgtgtccaagggatgggctggcacctcttggaccaggcttaccaccagggcccttctctgaagccccagtctgaccggcctgctgctgggaatccccctctgcccccacactaacctctgctggggctgagccagggcgcgtcggacagtcagggcgacccagccagggcgaccgttggccccgctcctatggggcagcagggaccgacgtcagcagggtggggcgggcacccgagtggtatgccccgccctgccccgcctgcccgccctggtggccgtctgggggcgacaagtcctgagagaaccagacggaagcgcgctgggactgacacgtggacttgggcggtgctgcccgggtgggtcagcctgggctgggaggcagccccgggacacagctgtgcccacgccgtctgagcaccccaagcccgatgcagccacccccagacgaggcccgcagggacatggccggggacacccagtggtccaggtgtggcgggggtgaggggagggggggtgggagcggtggagatggggccgtggggagggagctgagatactgccacgtgggacgatgctaggtggggagggctgagctgggcgggctcctctggctgtggggccccctgtgttccttgtgggaggtggaaggaagtgagtgccctgtccttcctccctgccatgagattccaggaccggacctggcaagtgccctatcccagccagtgttcctggggctcttccagg

SEQ ID NO: 194 - DNA, 1063 bp, Artificial Sequence
  Bisulfite converted genomic DNA from chr2061583436-61584498
ttagaatttggtttgtgatgggtttttaggatattttttgtttaggaatttaggggttaatgataatttttataggtatttgttgagaagatatgggttagcgttggatttgtttagtttttttaggtttggtttaagggtttaggagtggatttatttttgtttcgaaagatttattttaaaaattagtgattttgtatttttattttttttgtatttggttaagattttatttttgattttatttgatcggtaaggtagggattgttattattttttattttatagatagaggatagacggttttatggtttcgtgtcgtgtttaagggatgggttggtattttttggattaggtttattattagggtttttttttgaagttttagtttgatcggtttgttgttgggaatttttttttgtttttatattaatttttgttggggttgagttagggcgcgtcggatagttagggcgatttagttagggcgatcgttggtttcgtttttatggggtagtagggatcgacgttagtagggtggggcgggtattcgagtggtatgtttcgttttgtttcgtttgttcgttttggtggtcgtttgggggcgataagttttgagagaattagacggaagcgcgttgggattgatacgtggatttgggcggtgttgttcgggtgggttagtttgggttgggaggtagtttcgggatatagttgtgtttacgtcgtttgagtattttaagttcgatgtagttatttttagacgaggttcgtagggatatggtcggggatatttagtggtttaggtgtggcgggggtgaggggagggggggtgggagcggtggagatggggtcgtggggagggagttgagatattgttacgtgggacgatgttaggtggggagggttgagttgggcgggtttttttggttgtggggttttttgtgttttttgtgggaggtggaaggaagtgagtgttttgttttttttttttgttatgagattttaggatcggatttggtaagtgttttattttagttagtgtttttggggtttttttagg

SEQ ID NO: 195 - DNA, 26 bp, Artificial Sequence
  F1 Primer for Bisulfite converted genomic DNA from chr2061583436-61584498
tttggttaagattttatttttgattt

SEQ ID NO: 196 - DNA, 21 bp, Artificial Sequence
  R1 Primer for Bisulfite converted genomic DNA from chr2061583436-61584498
ctaacccacccgaacaacacc

SEQ ID NO: 197 - DNA, 20 bp, Artificial Sequence
  F2 Primer for Bisulfite converted genomic DNA from chr2061583436-61584498
gttcgttttggtggtcgttt

SEQ ID NO: 198 - DNA, 20 bp, Artificial Sequence
  R2 Primer for Bisulfite converted genomic DNA from chr2061583436-61584498
aaccctccccacctaacatc

(19) United States
(12) Patent Application Publication
(10) Pub. No.: US 2022/0364187 A1
(43) Pub. Date: Nov. 17, 2022

(54) DETECTING, PREDICTING SEVERITY OF, AND/OR PREDICTING TREATMENT RESPONSE TO RESPIRATORY VIRUS INFECTION

(71) Applicants: WISCONSIN ALUMNI RESEARCH FOUNDATION, MADISON, WI (US); ALBANY MEDICAL COLLEGE, ALBANY, NY (US)

(72) Inventors: Reid S. ALISCH, Prairie du Sac, WI (US); Kirk J. HOGAN, Madison, WI (US); Andy MADRID, Madison, WI (US); Ariel JAITOVICH, Albany, NY (US)

(73) Assignees: WISCONSIN ALUMNI RESEARCH FOUNDATION, MADISON, WI (US); ALBANY MEDICAL COLLEGE, ALBANY, NY (US)

(21) Appl. No.: 17/746,033

(22) Filed: May 17, 2022

Related U.S. Application Data
(60) Provisional application No. 63/189,479, filed on May 17, 2021.

(51) Int. Cl. C12Q 1/6888; G01N 33/569; C12Q 1/686

(52) U.S. Cl. CPC C12Q 1/6888; G01N 33/56983; C12Q 1/686; C12Q 2600/154; C12Q 2600/112; C12Q 2600/158; C12Q 2600/118

(57) ABSTRACT

Methods of detecting, predicting severity of, and/or predicting treatment response to respiratory virus infection in a sample obtained from a subject. The methods include assaying a methylation state of a marker in a sample obtained from a subject and identifying the subject as having respiratory virus infection, a likelihood of severe outcomes of respiratory infection, and/or a likelihood of treatment response depending on the methylation state of the marker. The markers can include bases (DMP) in differentially methylated regions (DMR) as provided herein.

### CROSS-REFERENCE TO RELATED APPLICATIONS

Priority is claimed to U.S. Provisional Application 63/189,479, filed May 17, 2021, which is incorporated herein by reference in its entirety.

### STATEMENT REGARDING FEDERALLY SPONSORED RESEARCH

This invention was made with government support under AG066179, AG062715 and HL130704 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. The ASCII copy, created on May 6, 2022, is named USPTO—220517—Patent_Application—P210192US02—SEQUENCE_LISTING_ST25.txt and is 152,329 bytes in size.

### FIELD OF THE INVENTION

Provided herein is technology for detecting, predicting severity of, and/or predicting treatment response to respiratory virus infection, such as SARS-CoV-2, and particularly, but not exclusively, to methods, compositions, and related uses for detecting, predicting severity of, and/or predicting treatment response to respiratory virus infection, such as SARS-CoV-2 infection.

### BACKGROUND

About 2.5 million deaths worldwide have been attributed to COVID-19, primarily arising from acute respiratory distress syndrome (ARDS). The clinical course of SARS-CoV-2 (severe acute respiratory syndrome coronavirus 2) infection is highly variable, ranging from an asymptomatic state to life-threatening infection¹⁻⁴. Recent evidence indicates that disease severity predominantly depends on host factors⁵⁻¹⁰, supporting the need to better resolve individual responses at the molecular level. The multi-omic profile of COVID-19 patients in association with disease severity has been described¹⁰⁻¹². Analysis of mRNA sequencing from circulating leukocytes identified multiple expressed genes associated with worse outcomes^{10,11,13}.

Because almost every cell in an individual shares an identical genomic sequence, distinct cellular phenotypes are established and maintained by epigenetic mechanisms^{14,15}, including DNA, histone and chromatin modifications and non-coding RNA expression¹⁶. Although DNA 5′-C-phosphate-G-3′ (CpG) methylation is relatively stable¹⁵⁻¹⁸, it is highly sensitive to age and environmental factors^{16,19-23}. Critically ill patients exhibit altered circulating blood DNA methylation profiles^{24,25}. Epigenetic marks affect gene expression profiles and increase individual vulnerability to viral infections²⁶. For example, modulators of host-pathogen interactions including interferons are epigenetically regulated^{27,28}, and DNA methylation has been shown to underpin antigen-presentation following MERS-CoV infection²⁷⁻²⁹. To date, it is unknown if patients with worse outcomes and distinct transcriptomes^{11,30,31} may be further distinguished by patterns of differential methylation. Such data may carry strong potential to illuminate mechanisms underlying COVID-19-associated gene expression and outcomes^{32,33}, and may facilitate the identification of subphenotypes likely to benefit from specific interventions³⁴⁻³⁶. For example, immune modulating drugs such as corticosteroids, that are beneficial in COVID-19 patients^{7,37-39}, interact with gene expression-response elements throughout the genome. Resolution of the differential methylome in COVID-19 patients offers insights into COVID-19 pathogenesis, susceptibility, diagnosis and prognosis.

Technology for detecting, predicting severity of, and/or predicting treatment response to respiratory virus infection, such as SARS-CoV-2 infection, or COVID-19 is needed.

### SUMMARY OF THE INVENTION

The objects and advantages of the invention will appear more fully from the following detailed description of the preferred embodiment of the invention made in conjunction with the accompanying drawings.

Methylated DNA has been studied as a potential class of biomarkers in a number of diseases. In many instances, DNA methyltransferases add a methyl group to DNA at cytosine-phosphate-guanine (CpG) island sites as an epigenetic control of gene expression. In a biologically attractive mechanism, acquired methylation events in promoter regions of tumor suppressor genes may silence expression, thus contributing to oncogenesis. DNA methylation may be a more chemically and biologically stable diagnostic tool than RNA or protein expression (Laird (2010) Nat Rev Genet 11: 191-203). Furthermore, in cancers like sporadic colon cancer, methylation markers offer excellent specificity and are more broadly informative and sensitive than are individual DNA mutations (Zou et al (2007) Cancer Epidemiol Biomarkers Prev 16: 2686-96).

Analysis of CpG islands has yielded important findings when applied to animal models and human cell lines. For example, Zhang and colleagues found that amplicons from different parts of the same CpG island may have different levels of methylation (Zhang et al. (2009) PLoS Genet 5: e1000438). Methylation levels were distributed bi-modally between highly methylated and unmethylated sequences, further supporting the binary switch-like pattern of DNA methyltransferase activity (Zhang et al. (2009) PLoS Genet 5: e1000438).

Several methods are available to search for novel methylation markers. There are three basic approaches. The first employs digestion of DNA by restriction enzymes which recognize specific methylated sites, followed by several possible analytic techniques which provide methylation data limited to the enzyme recognition site or the primers used to amplify the DNA in quantification steps (such as methylation-specific PCR; MSP). A second approach enriches methylated fractions of genomic DNA using anti-bodies directed to methyl-cytosine or other methylation-specific binding domains followed by microarray analysis or sequencing to map the fragment to a reference genome. This approach does not provide single nucleotide resolution of all methylated sites within the fragment. A third approach begins with bisulfite treatment of the DNA to convert all unmethylated cytosines to uracil, followed by various methylation assay procedures (e.g., microarray-based and sequencing analysis).

Provided herein is technology for diagnosing, predicting severity of, and/or predicting treatment response to respiratory virus infection, such as SARS-CoV-2 infection, and particularly, but not exclusively, to methods, compositions, and related uses for diagnosing, predicting severity of, and/or predicting treatment response to respiratory virus infection, such as SARS-CoV-2 infection.

As described in the Examples, experiments identified 1505 differentially methylated regions (DMRs) for discriminating COVID-19 positive patient's DNA from healthy pre-pandemic controls (Table 3). In addition, 254 DMRs were identified for discriminating DNA of COVID-19 positive patients from non-COVID respiratory patients (Table 6), in which 47 DMRs are shared with DMRs identified in the comparison of COVID-19 patients vs. healthy controls.

Additional experiments were directed toward identifying markers capable of distinguishing COVID-19 severity in terms of GRAM risk scores40 and mortality. It was found that worse GRAM scores were associated with 19 DMRs comprising 145 differentially methylated positions (DMPs) in 18 genes (Table 13). Mortality was associated with 18 DMRs comprising 113 DMPs in 17 genes (Tables 14 and 15).

Additional experiments were directed toward identifying markers capable of distinguishing responders and non-responders to corticosteroids within COVID-19 patients. This approach identified a large number of DMRs (Table 16) and 8 DMPs (Table 17).

As described herein, the technology provides a number of methylated DNA markers and subsets thereof (e.g., sets of 2, 3, 4, 5, 6, 7, 8, 9, 10, 11, 12, 13, 14, 15, 16, 17, 18, 19, 20, 21, or more markers) with high discrimination for diagnosing, predicting severity of, and/or predicting treatment response to respiratory virus infection, such as SARS-CoV-2 infection.

In some embodiments, the technology is related to assessing the presence of and methylation state of one or more of the markers identified herein in a biological sample (e.g., blood sample). These markers comprise one or more differentially methylated regions (DMRs) or differentially methylated positions (DMPs) as discussed herein, e.g., as provided in Tables 3, 6, 13, 14, 15, 16, 17A and 17B. Methylation state is assessed in embodiments of the technology. As such, the technology provided herein is not restricted in the method by which a gene's methylation state is measured. For example, in some embodiments the methylation state is measured by a genome scanning method. For example, one method involves restriction landmark genomic scanning (Kawai et al. (1994) Mol. Cell. Biol. 14: 7421-7427) and another example involves methylation-sensitive arbitrarily primed PCR (Gonzalgo et al. (1997) Cancer Res. 57: 594-599). In some embodiments, changes in methylation patterns at specific CpG sites are monitored by digestion of genomic DNA with methylation-sensitive restriction enzymes followed by Southern analysis of the regions of interest (digestion-Southern method). In some embodiments, analyzing changes in methylation patterns involves a PCR-based process that involves digestion of genomic DNA with methylation-sensitive restriction enzymes prior to PCR amplification (Singer-Sam et al. (1990) Nucl. Acids Res. 18: 687). In addition, other techniques have been reported that utilize bisulfite treatment of DNA as a starting point for methylation analysis. These include methylation-specific PCR (MSP) (Herman et al. (1992) Proc. Natl. Acad. Sci. USA 93: 9821-9826) and restriction enzyme digestion of PCR products amplified from bisulfite-converted DNA (Sadri and Hornsby (1996) Nucl. Acids Res. 24: 5058-5059; and Xiong and Laird (1997) Nucl. Acids Res. 25: 2532-2534). PCR techniques have been developed for detection of gene mutations (Kuppuswamy et al. (1991) Proc. Natl. Acad. Sci. USA 88: 1143-1147) and quantification of allelic-specific expression (Szabo and Mann (1995) Genes Dev. 9: 3097-3108; and Singer-Sam et al. (1992) PCR Methods Appl. 1: 160-163). Such techniques use internal primers, which anneal to a PCR-generated template and terminate immediately 5′ of the single nucleotide to be assayed. Methods using a “quantitative Ms-SNuPE assay” as described in U.S. Pat. No. 7,037,650 are used in some embodiments. Methylation arrays, such as the Infinium HD Methylation Assay (Pidsley et al. (2016) Genome Biol. 17:208), are used in some embodiments. In other embodiments, direct sequencing including next generation sequencing methods are used to assess the methylation status of patient DNA comprising, for example, Sanger sequencing (Sanger F et al (1977). DNA sequencing with chain-terminating inhibitors. Proc. Natl. Acad. Sci. U.S.A. 74 (12): 5463-7), Illumina NovSeq sequencing (Raine A et al (2018) Data quality of whole genome bisulfite sequencing on Illumina platforms. PLoS ONE 13(4): e0195972), PacBio sequencing (Eid J et al (2009) Real-time DNA sequencing from single polymerase molecules. Science. 323: 133-138), 454 sequencing (Margulies M et al (2005). Genome Sequencing in Open Microfabricated High Density Picoliter Reactors. Nature. 437(7057): 376-380), Ion Torrent sequencing (Rothberg J M et al (2011): An integrated semiconductor device enabling non-optical genome sequencing. Nature. 475 (7356): 348-352), and Oxford Nanopore sequencing (Eisenstein, M (2012). Oxford Nanopore announcement sets sequencing sector abuzz. Nat Biotechnol 30, 295-296), and other next generation sequencing platforms.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated and the resulting methylation-specific sequence is determined using either a real-time PCR or other exponential amplification, e.g., a QUARTS assay (e.g., as provided by U.S. Pat. No. 8,361,720; and U.S. Pat. Appl. Pub. Nos. 2012/0122088 and 2012/0122106, incorporated herein by reference).

For example, in some embodiments methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amount of methylated DNAs relative to the total amount of DNAs in the population, e.g., (number of methylated DNAs)/(the number of methylated DNAs+number of unmethylated DNAs)×100.

Some embodiments of the invention comprise determining a methylation level of any one or more CpG sites in any one or more genomic regions of a genome of a subject, wherein the one or more genomic regions comprise one or more of: positions 40024971-40025415 of chromosome 1; positions 63249197-63249213 of chromosome 1; positions 95698827-95699097 of chromosome 1; positions 154127462-154128443 of chromosome 1; positions 162467080-162467363 of chromosome 1; positions 27301195-27301943 of chromosome 2; positions 47382287-47382903 of chromosome 2; positions 121223534-121223964 of chromosome 2; positions 42756397-42757171 of chromosome 5; positions 102898463-102898733 of chromosome 5; positions 110062384-110062618 of chromosome 5; positions 138210550-138211184 of chromosome 5; positions 180257691-180257804 of chromosome 5; positions 28058187-28059208 of chromosome 6; positions 28829283-28829674 of chromosome 6; positions 32164503-32165200 of chromosome 6; positions 168393930-168394160 of chromosome 6; positions 965379-965534 of chromosome 7; positions 151433178-151433561 of chromosome 7; positions 16859295-16860121 of chromosome 8; positions 144635260-144635610 of chromosome 8; positions 76803669-76803925 of chromosome 10; positions 2334892-2335053 of chromosome 11; positions 2891077-2891118 of chromosome 11; positions 15037755-15039432 of chromosome 12; positions 49107116-49108131 of chromosome 13; positions 110438578-110439234 of chromosome 13; positions 112861499-112861518 of chromosome 13; positions 72053146-72053361 of chromosome 14; positions 104394430-104394831 of chromosome 14; positions 101389272-101389394 of chromosome 15; positions 3355951-3356149 of chromosome 16; positions 49563759-49564462 of chromosome 16; positions 67034309-67034882 of chromosome 16; positions 75681737-75682004 of chromosome 16; positions 75568999-75569749 of chromosome 16; positions 6797466-6797771 of chromosome 17; positions 8066669-8067323 of chromosome 17; positions 11784246-11785188 of chromosome 19; positions 52390810-52391789 of chromosome 19; positions 5485144-5486007 of chromosome 20; and positions 61583686-61584248 of chromosome 20.

The determining can comprise determining the methylation level of any 1 or more, 2 or more, 3 or more, 4 or more, 5 or more, 6 or more, 7 or more, 8 or more, 9 or more, or 10 or more CpG sites in any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more of the genomic regions.

The determining can comprise determining the methylation level of each CpG site in any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more of the genomic regions.

The determining can comprise determining the methylation level of any 1 or more, 2 or more, 3 or more, 4 or more, 5 or more, 6 or more, 7 or more, 8 or more, 9 or more, or 10 or more CpG sites in each of the genomic regions.

The determining comprises determining the methylation level of each CpG site in each of the genomic regions.

In some embodiments, the methylation level is determined for no more than 800,000 CpG sites, no more than 700,000 CpG sites, no more than 600,000 CpG sites, no more than 500,000 CpG sites, no more than 400,000 CpG sites, no more than 300,000 CpG sites, no more than 250,000 CpG sites, no more than 200,000 CpG sites, no more than 150,000 CpG sites, no more than 100,000 CpG sites, no more than 75,000 CpG sites, no more than 50,000 CpG sites, no more than 25,000 CpG sites, no more than 10,000 CpG sites, no more than 5,000 CpG sites, no more than 2,500 CpG sites, no more than 1,000 CpG sites, no more than 750 CpG sites, no more than 500 CpG sites, no more than 400 CpG sites, no more than 300 CpG sites, no more than 200 CpG sites, no more than 100 CpG sites, no more than 75 CpG sites, no more than 50 CpG sites, or no more than 25 CpG sites in the genome of the subject.

In some embodiments, the subject is a subject presenting with respiratory illness. In some embodiments, the subject is a subject infected or suspected of being infected with coronavirus. In some embodiments, the subject is a subject infected or suspected of being infected with SARS-CoV-2. In some embodiments, the subject is a subject diagnosed with SARS-CoV-2 infection.

In preferred embodiments, the subject is a mammal. In further embodiments, the subject is a human.

In some embodiments, the determining comprises: treating genomic DNA from the subject with bisulfite to generate bisulfite-treated genomic DNA; amplifying the bisulfite-treated genomic DNA using primers specific for a portion of the bisulfite-treated genomic DNA comprising the one or more genomic regions; and measuring the methylation level of the one or more CpG sites in the one or more genomic regions. The phrase “primers specific for a portion” of a given nucleic acid means that the primer is configured (has a sequence and length) to hybridize within that portion of the nucleic acid (e.g., DNA). “Portion” as used herein with reference to a given nucleic acid (e.g., DNA) refers to a region of the nucleic acid. Some embodiments further comprise isolating the genomic DNA from the subject or a biological sample from the subject. In some embodiments, the biological sample is a blood sample. In some embodiments, the portion of the bisulfite-treated genomic DNA has a length less than 1000 bases, less than 950 bases, less than 900 bases, less than 850 bases, less than 800 bases, less than 750 bases, less than 700 bases, less than 650 bases, less than 600 bases, less than 550 bases, less than 500 bases, less than 450 bases, less than 400 bases, 350 bases, less than 300 bases, less than 250 bases, less than 200 bases, less than 150 bases, less than 100 bases, less than 75 bases, or less than 50 bases. In some embodiments, an amplicon resulting from the amplification has a length less than 1000 bases, less than 950 bases, less than 900 bases, less than 850 bases, less than 800 bases, less than 750 bases, less than 700 bases, less than 650 bases, less than 600 bases, less than 550 bases, less than 500 bases, less than 450 bases, less than 400 bases, 350 bases, less than 300 bases, less than 250 bases, less than 200 bases, less than 150 bases, less than 100 bases, less than 75 bases, or less than 50 bases. In some embodiments, an amplicon resulting from the amplification has a length of at least 100 bases, at least 150 bases, or at least 200 bases and less than 400 bases, less than 350 bases or less than 300 bases. In some versions, the primers can comprise sequencing adapters, such as next-generation sequencing adapters.

Exemplary primers and an exemplary primer-design protocol are provided in the following examples section and Table 20.

In some embodiments, the methylation level is measured by methylation-specific PCR, quantitative methylation-specific PCR, methylation-sensitive DNA restriction enzyme analysis, or bisulfite genomic sequencing PCR, or quantitative bisulfite pyrosequencing.

In some embodiments, the one or more genomic regions comprise any one or more of: positions 40024971-40025415 of chromosome 1; positions 95698827-95699097 of chromosome 1; positions 162467080-162467363 of chromosome 1; positions 27301195-27301943 of chromosome 2; positions 47382287-47382903 of chromosome 2; positions 42756397-42757171 of chromosome 5; positions 102898463-102898733 of chromosome 5; positions 138210550-138211184 of chromosome 5; positions 28829283-28829674 of chromosome 6; positions 168393930-168394160 of chromosome 6; positions 965379-965534 of chromosome 7; positions 2334892-2335053 of chromosome 11; positions 2891077-2891118 of chromosome 11; positions 110438578-110439234 of chromosome 13; positions 101389272-101389394 of chromosome 15; positions 3355951-3356149 of chromosome 16; positions 11784246-11785188 of chromosome 19; positions 52390810-52391789 of chromosome 19; and positions 61583686-61584248 of chromosome 20. In some embodiments, the one or more CpG sites are located at any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 30 or more, 35 or more, 40 or more, 45 or more, 50 or more, 55 or more, 60 or more, 65 or more, 70 or more, or 75 or more of the genomic positions shown in Table 15. In some embodiments, the method further comprises determining from the methylation level of the one or more CpG sites a likelihood of developing critical illness comprising at least one of admission to an intensive care unit, invasive ventilation, and death.

In some embodiments, the one or more genomic regions comprise any one or more of: positions 63249197-63249213 of chromosome 1; positions 27301195-27301943 of chromosome 2; positions 121223534-121223964 of chromosome 2; positions 110062384-110062618 of chromosome 5; positions 180257691-180257804 of chromosome 5; positions 28058187-28059208 of chromosome 6; positions 32164503-32165200 of chromosome 6; positions 151433178-151433561 of chromosome 7; positions 16859295-16860121 of chromosome 8; positions 144635260-144635610 of chromosome 8; positions 76803669-76803925 of chromosome 10; positions 112861499-112861518 of chromosome 13; positions 72053146-72053361 of chromosome 14; positions 104394430-104394831 of chromosome 14; positions 49563759-49564462 of chromosome 16; positions 67034309-67034882 of chromosome 16; positions 75568999-75569749 of chromosome 16; and positions 6797466-6797771 of chromosome 17. In some embodiments, the subject is a subject infected with SARS-CoV-2 and the method further comprises determining from the methylation level of the one or more CpG sites a likelihood of dying from the SARS-CoV-2 infection.

In some embodiments, the one or more genomic regions comprise one or more of: positions 154127462-154128443 of chromosome 1; positions 15037755-15039432 of chromosome 12; positions 49107116-49108131 of chromosome 13; positions 75681737-75682004 of chromosome 16; positions 8066669-8067323 of chromosome 17; and positions 5485144-5486007 of chromosome 20. In some embodiments, the one or more CpG sites are located at any 1 or more, 2 or more, 3 or more, 4 or more, 5 or more, 6 or more, 7 or more, or all of the genomic positions shown in Table 17A. In some embodiments, the subject is a subject infected with SARS-CoV-2, and the method further comprises determining from the methylation level of the one or more CpG sites a likelihood of the subject responding to treatment. Some embodiments further comprise treating the subject. In some embodiments, the treating comprising treating with a steroid, such as a corticosteroid. Exemplary corticosteroids include bethamethasone, prednisone, prednisolone, triamcinolone, methylprednisolone, dexamethasone, hydrocortisone, cortisone, ethamethasoneb, budesonide, and fludrocortisone. In some embodiments, the treating comprises administering an anti-SARS-CoV-2 antibody. In some embodiments, the anti-SARS-CoV-2 antibody is a polyclonal antibody (e.g., provided by convalescent plasma). In some embodiments, the anti-SARS-CoV-2 antibody is a monoclonal antibody (e.g., bamlanivimab or casirivimab).

Also provided herein are compositions and kits and systems for practicing the methods. For example, in some embodiments, reagents (e.g., primers, probes) specific for one or more markers are provided alone or in sets (e.g., sets of primers pairs for amplifying a plurality of markers). Additional reagents for conducting a detection assay may also be provided (e.g., enzymes, buffers, positive and negative internal and external controls for conducting QuARTS, PCR, sequencing, bisulfite, calibrants or other assays). In some embodiments, the kits containing one or more reagents necessary, sufficient, or useful for conducting a method are provided. Also provided are reactions mixtures containing the reagents, and instructions for use of the reagents. Further provided are master mix reagent sets containing a plurality of reagents that may be added to each other and/or to a test sample to complete a reaction mixture.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation state (e.g., of any one or more DMRs and/or DMPs provided in any one or more of Tables 3, 6, 13, 14, 15, 16, 17A and 17B); comparing methylation states (e.g., of any one or more DMRs and/or DMPs provided in any one or more of Tables 3, 6, 13, 14, 15, 16, 17A and 17B); generating standard curves; determining a Ct value; calculating a fraction, frequency, or percentage of methylation (e.g., of any one or more DMRs and/or DMPs provided in any one or more of Tables 3, 6, 13, 14, 15, 16, 17A and 17B); identifying a CpG island; determining a specificity and/or sensitivity of an assay or marker; calculating an ROC curve and an associated AUC; sequence analysis; all as described herein or is known in the art.

In some embodiments, a microprocessor or computer uses methylation state data in an algorithm to diagnose, predict the severity of, and/or predicting treatment response to respiratory virus infection.

In some embodiments, a software or hardware component receives the results of multiple assays and determines a single value result to report to a user that indicates a diagnosis, a prediction of the severity of, and/or a prediction of treatment response to respiratory virus infection based on the results of the multiple assays (e.g., determining the methylation state of multiple DMRs and/or DMPs, e.g., as provided in any one or more of Tables 3, 6, 13, 14, 15, 16, 17A and 17B). Related embodiments calculate a risk factor based on a mathematical combination (e.g., a weighted combination, a linear combination) of the results from multiple assays, e.g., determining the methylation states of multiple markers (such as multiple DMRs and/or DMPs, e.g., as provided in any one or more of Tables 3, 6, 13, 14, 15, 16, 17A and 17B). In some embodiments, the methylation state of a DMR defines a dimension and may have values in a multidimensional space and the coordinate defined by the methylation states of multiple DMRs is a result, e.g., to report to a user, e.g., related to a diagnosis, prediction of the severity of, and/or prediction of the treatment response to respiratory virus infection.

Some embodiments comprise a storage medium and memory components. Memory components (e.g., volatile and/or nonvolatile memory) find use in storing instructions (e.g., an embodiment of a process as provided herein) and/or data (e.g., a work piece such as methylation measurements, sequences, and statistical descriptions associated therewith). Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship.

In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

Accordingly, provided herein is technology related to a method of diagnosing, predicting severity of, and/or predicting treatment response to respiratory virus infection in a sample obtained from a subject, the method comprising assaying a methylation state of a marker in a sample obtained from a subject (e.g., a blood sample, a saliva sample, a sputum sample, a bronchoalveolar lavage (BAL), a cerebrospinal fluid sample, a urine sample, a biopsy sample, a nasopharyngeal or oropharynx swab sample) and identifying the subject as having respiratory virus infection, a likelihood of severe outcomes of respiratory infection, and/or a likelihood of treatment response (e.g., with steroids, such as corticosteroids) when the methylation state of the marker is different than a methylation state of the marker assayed in a subject that does not have respiratory virus infection, does not have severe outcomes of respiratory infection, and/or does not have response to treatment, wherein the marker comprises a base (DMP) in a differentially methylated region (DMR) selected from a group consisting of any one or more of the DMRs and/or DMPs provided in any one or more of Tables 3, 6, 13, 14, 15, 16, 17A and 17B.

In some embodiments, the methylation state of DMRs and/or DMPs in Table 3 can be determined and, optionally, used to diagnose respiratory disease infection. In some embodiments, the methylation state of DMRs and/or DMPs in Table 3 can be determined and used to diagnose SARS-CoV-2 infection. In some embodiments, the methylation state of DMRs and/or DMPs in Table 3 can be determined and used to distinguish individuals with SARS-CoV-2 infection from healthy individuals. Any combination of DMRs and/or DMPs in Table 3 can be used in such embodiments.

In some embodiments, the methylation state of DMRs and/or DMPs that are shown as being differentially methylated in Table 21 can be determined. The DMRs and/or DMPs can optionally be used as markers to identify or predict patients having lasting effects of SARS-CoV-2 (referred to in the art as “long COVID” or Post-Acute Sequelae of SARS-CoV-2 Infection (PASC)) or other respiratory infection. Any combination of DMRs and/or DMPs that are shown as being differentially methylated in Table 21 can be used in such embodiments.

In some embodiments, the methylation state of DMRs and/or DMPs in Table 6 can be determined and, optionally, used to diagnose respiratory disease infection. In some embodiments, the methylation state of DMRs and/or DMPs in Table 6 can be determined and used to diagnose SARS-CoV-2 infection. In some embodiments, the methylation state of DMRs and/or DMPs in Table 6 can be determined and used to distinguish individuals with SARS-CoV-2 infection from individuals with other respiratory diseases. Any combination of DMRs and/or DMPs in Table 6 can be used in such embodiments.

In some embodiments, the methylation state of DMRs and/or DMPs in Table 13 can be determined and, optionally, used to predict the severity of respiratory virus infection. In some embodiments, the methylation state of DMRs and/or DMPs in Table 13 can be determined and used to predict the severity of SARS-CoV-2 infection. In some embodiments, the methylation state of DMRs and/or DMPs in Table 13 can be determined and used to predict the likelihood of developing critical illness from SARS-CoV-2 infection, wherein the critical illness comprises admission to an intensive care unit, invasive ventilation, or death. Any combination of DMRs and/or DMPs in Table 13 can be used in such embodiments.

In some embodiments, the methylation state of DMRs and/or DMPs in Tables 14 and 15 can be determined and, optionally, used to predict the severity of respiratory virus infection. In some embodiments, the methylation state of DMRs and/or DMPs in Tables 14 and 15 can be determined and used to predict the severity of SARS-CoV-2 infection. In some embodiments, the methylation state of DMRs and/or DMPs in Tables 14 and 15 can be determined and used to predict the likelihood of dying from SARS-CoV-2 infection. Any combination of DMRs and/or DMPs in Tables 14 and 15 can be used in such embodiments.

In some embodiments, the methylation state of DMRs and/or DMPs in Tables 16 and 17 can be determined and, optionally, used to predict the response to treatment of respiratory virus infection. In some embodiments, the methylation state of DMRs and/or DMPs in Tables 16 and 17 can be determined and used to predict the response to treatment of SARS-CoV-2 infection. In some embodiments, the methylation state of DMRs and/or DMPs in Tables 16 and 17 can be determined and used to predict the response to treatment of SARS-CoV-2 infection with steroids. In some embodiments, the methylation state of DMRs and/or DMPs in Tables 16 and 17 can be determined and used to predict the response to treatment of SARS-CoV-2 infection with corticosteroids. Any combination of DMRs and/or DMPs in Tables 16 and 17 can be used in such embodiments.

Some embodiments provide methods comprising assaying a plurality of markers. In some embodiments, the plurality of markers comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, or at least 150 markers. In some embodiments, the plurality of markers comprise fewer than 3, fewer than 4, fewer than 5, fewer than 6, fewer than 7, fewer than 8, fewer than 9, fewer than 10, fewer than 11, fewer than 12, fewer than 13, fewer than 14, fewer than 15, fewer than 16, fewer than 17, fewer than 18, fewer than 19, fewer than 20 fewer than 30, fewer than 40, fewer than 50, fewer than 60, fewer than 70, fewer than 80, fewer than 90, fewer than 100, fewer than 110, fewer than 120, fewer than 130, fewer than 140, or fewer than 150 markers.

The technology is not limited in the methylation state assessed. In some embodiments assessing the methylation state of the marker in the sample comprises determining the methylation state of one base. In some embodiments, assaying the methylation state of the marker in the sample comprises determining the extent of methylation at a plurality of bases. Moreover, in some embodiments the methylation state of the marker comprises an increased methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

Furthermore, in some embodiments the marker is a region of 100 or fewer bases, the marker is a region of 500 or fewer bases, the marker is a region of 1000 or fewer bases, the marker is a region of 5000 or fewer bases, or, in some embodiments, the marker is one base.

The technology is not limited by sample type. For example, in some embodiments the sample is a blood sample (e.g., plasma, serum, whole blood), a stool sample, a tissue sample (e.g., lung tissue sample), an excretion, or a urine sample.

Furthermore, the technology is not limited in the method used to determine methylation state. In some embodiments the assaying comprises using methylation specific polymerase chain reaction, nucleic acid sequencing, mass spectrometry, methylation specific nuclease, mass-based separation, or target capture. In some embodiments, the assaying comprises use of a methylation specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc. In some embodiments, the technology uses array-based methylation analysis.

The technology provides reagents for detecting the methylation state of a DMR and/or DMP, e.g., in some embodiments are provided a set of oligonucleotides comprising the sequences of the oligonucleotides provided herein. In some embodiments are provided an oligonucleotide comprising a sequence complementary to a chromosomal region having a base in a DMR, e.g., an oligonucleotide sensitive to methylation state of a DMR and/or DMP.

Kit embodiments are provided, e.g., a kit comprising a bisulfite reagent; and a control nucleic acid comprising a sequence from a DMR and/or DMP selected from a group consisting of any one or more of Tables 3, 6, 13, 14, 15, 16, 17A and 17B and having a methylation state associated with a subject who has or does not have a health condition as described herein. In some embodiments, kits comprise a bisulfite reagent and an oligonucleotide as described herein. In some embodiments, kits comprise a bisulfite reagent; and a control nucleic acid comprising a sequence from a DMR selected from a group consisting of any one or more DMRs in any one or more of Tables 3, 6, 13, 14, 15, 16, 17A and 17B and having a methylation state as described in the following examples. Some kit embodiments comprise a sample collector for obtaining a sample from a subject (e.g., blood sample); reagents for isolating a nucleic acid from the sample; a bisulfite reagent; and an oligonucleotide as described herein.

The technology is related to embodiments of compositions (e.g., reaction mixtures). In some embodiments are provided a composition comprising a nucleic acid comprising a DMR and/or a DMP and a bisulfite reagent. Some embodiments provide a composition comprising a nucleic acid comprising a DMR and/or a DMP and an oligonucleotide as described herein. Some embodiments provide a composition comprising a nucleic acid comprising a DMR and/or a DMP a methylation-sensitive restriction enzyme. Some embodiments provide a composition comprising a nucleic acid comprising a DMR and/or a DMP and a polymerase.

Additional related method embodiments are provided for diagnosing, predicting the severity of, and/or predicting the treatment response to respiratory virus infection in a sample obtained from a subject (e.g., blood sample), e.g., a method comprising determining a methylation state of a marker in the sample comprising a base in any one or more DMRs in any one or more of Tables 3, 6, 13, 14, 15, 16, 17A and 17B); comparing the methylation state of the marker from the subject sample to a methylation state of the marker from a control sample from a subject; and determining a confidence interval and/or a p value of the difference in the methylation state of the subject sample and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001. Some embodiments of methods provide steps of reacting a nucleic acid comprising a DMR and/or DMP with a bisulfite reagent to produce a bisulfite-reacted nucleic acid; sequencing the bisulfite-reacted nucleic acid to provide a nucleotide sequence of the bisulfite-reacted nucleic acid; comparing the nucleotide sequence of the bisulfite-reacted nucleic acid with a nucleotide sequence of a nucleic acid comprising the DMR and/or DMP from a negative and/or a positive control subject to identify differences in the two sequences; and identifying the subject as having a diagnosis or prognosis when a difference is present.

Systems for diagnosing, predicting the severity of, and/or a predicting treatment response to respiratory virus infection in a sample obtained from a subject are provided by the technology. Exemplary embodiments of systems include two or more of, e.g., a nucleic acid isolation and quantification component, a nucleic acid amplification component, a nucleic acid detection component, an analysis component configured to determine the methylation state of a sample, a software component configured to compare the methylation state of the sample with a control sample or a reference sample methylation state recorded in a database, and an alert component configured to alert a user of a diagnostic or prognostic methylation state. An alert is determined in some embodiments by a software component that receives the results from multiple assays (e.g., determining the methylation states of multiple markers, e.g., DMRs and/or DMPs as provided in Tables 3, 6, 13, 14, 15, 16, 17A and 17B) and calculating a value or result to report based on the multiple results. Some embodiments provide a database of weighted parameters associated with each DMR provided herein for use in calculating a value or result and/or an alert to report to a user (e.g., such as a physician, nurse, clinician, etc.). In some embodiments all results from multiple assays are reported and in some embodiments one or more results are used to provide a score, value, or result based on a composite of one or more results from multiple assays.

In certain embodiments, methods for characterizing a sample (e.g., blood sample) from a human patient are provided. For example, some embodiments comprise obtaining DNA from a sample of a human patient; assaying a methylation state of a DNA methylation marker comprising a base in a differentially methylated region (DMR) selected from a group consisting of any one or more DMRs from any one or more of Tables 3, 6, 13, 14, 15, 16, 17A and 17B; and comparing the assayed methylation state of the one or more DNA methylation markers with negative and/or positive methylation level references for the one or more DNA methylation markers.

Such methods are not limited to a particular type of sample from a human patient. In some embodiments, the sample is a blood sample. In some embodiments, the sample is a stool sample, a tissue sample, or a urine sample.

In some embodiments, the DNA methylation marker is a region of 100 or fewer bases. In some embodiments, the DNA methylation marker is a region of 500 or fewer bases. In some embodiments, the DNA methylation marker is a region of 1000 or fewer bases. In some embodiments, the DNA methylation marker is a region of 5000 or fewer bases. In some embodiments, the DNA methylation marker is one base.

In some embodiments, the assaying comprises using methylation specific polymerase chain reaction, nucleic acid sequencing, mass spectrometry, methylation specific nuclease, mass-based separation, or target capture.

In some embodiments, the assaying comprises use of a methylation specific oligonucleotide.

In certain embodiments, the technology provides methods for characterizing a sample obtained from a human patient. In some embodiments, such methods comprise determining a methylation state of a DNA methylation marker in the sample comprising a base in a DMR selected from a group consisting of any one or more DMRs from any one or more of Tables 3, 6, 13, 14, 15, 16, 17A and 17B; comparing the methylation state of the DNA methylation marker from the patient sample to a methylation state of the DNA methylation marker from a control sample from a control human subject; and determining a confidence interval and/or a p value of the difference in the methylation state of patient sample and the control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001.

In certain embodiments, the technology provides methods for characterizing a sample obtained from a human subject (e.g., blood sample), the method comprising reacting a nucleic acid comprising at least one DMR with a bisulfite reagent to produce a bisulfite-reacted nucleic acid; sequencing the bisulfite-reacted nucleic acid to provide a nucleotide sequence of the bisulfite-reacted nucleic acid; comparing the nucleotide sequence of the bisulfite-reacted nucleic acid with a nucleotide sequence of a nucleic acid comprising the DMR from a control subject.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Diagram of the entire cohort involved in the examples. Notice that while the hospitalized patients' cohort contributed 128 patients, 124 were part of the analyses due to inadequate quality of 4 samples, see diagram and details in the text.

FIGS. 2A-2E: Differential SARS-CoV-2 DNA methylation between blood samples from patients on hospital admission for COVID-19 compared to blood samples from healthy controls before the covid-19 pandemic. FIG. 2A: A box and whisker plot depicts the difference in mean global methylation level (y-axis) between Covid-19 patients and healthy controls (x-axis). Each black dot represents the mean methylation level of each participant. These results indicate that global mean methylation levels do not distinguish Covid-19 patients from healthy pre-pandemic controls. FIG. 2B: A Manhattan plot of DNA methylation regions shows the distribution of SARS-CoV-2-associated significantly differentially methylated regions (DMRs) across the genome by chromosome number. Hyper-methylated regions are displayed with a positive log 10 (P-value), and hypo-methylated regions are displayed with a negative log 10 (P-value). DMRs were ascertained as regions having at least 5 consecutive CpGs where >75% of the CpGs in the region had a false discovery rate (FDR) P-value <0.05, and all were either hyper-methylated or hypo-methylated. This approach identified 1,505 DMRs, that are displayed above and below the blue lines. Dots alternate colors to depict a change in chromosome. Sex chromosomes were excluded from analysis. These results indicate that 1,505 DNA regions are differentially methylated within days of SARS-Cov-2 infection. FIG. 2C: A pie chart showing the percent distribution of DMRs to standard genomic features. 5′UTR=5′ untranslated region′ 3′UTR=3′ untranslated region; TSS=transcription start site; TSS200=0-200 bp upstream of TSS; TSS1500=200-1500 bp upstream of TSS. In keeping with the known role of DNA methylation in regulation of gene expression, a preponderance of DMRs are located in gene promoter regions. FIG. 2D: Bar graphs of the top ten gene ontological (GO) biological processes related to the COVID-19 differentially methylated genes, ordered by statistical significance. The X-axis indicates the number of COVID-19 DMR-associated genes that contribute to each GO term. Bar color indicates the FDR P-value using a Fischer test. These results indicate that the observed DMRs occur in genes that participate in white blood cell activation and immune responses. FIG. 2E: Bar Graph of the top 10 disease ontological (DO) processes related to the COVID-19-associated differentially methylated genes, ordered by statistical significance. The X-axis indicates the number of COVID-19 DMR-associated genes contributing to each GO term. Bar color indicates the FDR P-value using a Fischer test. These results indicate that the observed DMRs occur in genes that participate in the pathogenesis of inflammatory and white blood cell disorders.

FIG. 3: No difference was observed in the gap between chronologic age and “epigenetic clock” age between COVID-19 patients and healthy pre-pandemic controls. The box plot depicts the difference in mean global methylation level (y-axis) between Covid-19 patients and healthy controls (1 and 0, respectively; x-axis).

FIGS. 4A-4D: DMRs in blood samples from COVID-19 patients on hospital admission are distinct from patients with non-COVID-19 respiratory illness in genes that participate in virus-related pathways and disorders. FIG. 4A: Box and whisker plot depicts the difference in mean global methylation level (y-axis) between COVID-19 and non-COVID-19 respiratory ill patients (x-axis). Each black dot represents the mean methylation level of each participant. These results indicate that global mean methylation levels do not distinguish COVID-19 from non-COVID-19 respiratory ill patients. FIG. 4B: Circos plot depicts genomic distribution of differentially methylated regions (DMRs) across the human genome. (Outer ring) Each chromosome is shown as a different color. The relative chromosome size is represented by the arc bar length. (Inner rings) Hyper-methylated DMRs are shown in red and hypo-methylated regions are shown in blue. Sex chromosomes were omitted from the analysis. These results indicate that 254 DNA differentially methylated regions distinguish SARS-Cov-2 infection from respiratory illness. FIG. 4C: Bar Graph of the top ten gene ontological (GO) biological processes related to the SARS-CoV-2-associated differentially methylated genes, ordered by statistical significance. The X-axis indicates the number of SARS-CoV-2 DMR-associated genes that contribute to each GO term. Bar color indicates the FDR P-value using a Fischer test. These results indicate that the observed DMRs occur in genes that participate in inflammatory and host-defense processes. FIG. 4D: Bar Graph of the top ten disease ontological (DO) processes related to the SARS-CoV-2-associated differentially methylated genes, ordered by statistical significance. The X-axis indicates the number of SARS-CoV-2 DMR-associated genes that contribute to each DO term. BAR color indicates the FDR P-value using a Fischer test. These results indicate that the observed DMRs occur in genes that participate in the pathology of influenza, other viral infections and inflammatory disorders.

FIGS. 5A-5D: Overlap of COVID-19 DMR-associated genes in blood. FIG. 5A: Venn diagram of the overlap of COVID-19 DMR-associated genes identified by comparison of DMRs between COVID-19 patients and healthy pre-pandemic controls, and DMRs between COVID-19 and non-COVID-19 respiratory illness patients on admission. Asterisks indicate overlap that is significant at P-value <0.001. Twenty-five of the 47 overlapping genes with DMRs encode proteins that participate in white blood cell viral defense, inflammation and immune responses. FIG. 5B: Ontology analysis of the 47 overlapping genes with DMRs indicate a role in viral defense mechanisms. FIGS. 5C and 5D: Relative positions of COVID-19 associated DMRs in the promoter region of OAS2 and IFI27 with a schematic depicted for each gene. The relative positions of probes measuring methylation levels of CpG sites annotated to each gene with their genomic 5′-3′ positions are provided (inset panel; x-axis) versus the −log 10 of the P-value (y-axis). The P-value <0.05 is displayed as a black line. Probes residing in a COVID-19 associated DMR are shown as hypo-methylation (blue dots) and hyper-methylation (red dots). Probes not meeting a P-value <0.05 at the individual CpG level are shown as hollow. These results indicate that the DMRs comprise a cluster of differentially methylated positions within days of SARS-Cov-2 infection.

FIGS. 6A-6B: DNA methylation is associated with COVID-19 outcomes. FIG. 6A: Volcano plot shows genes associated with dichotomized GRAM-risk scores, either hyper-methylated (purple) or hypo-methylated (green). FIG. 6B: DNA methylation levels at 77 differentially methylated positions (DMPs) correlate with disease severity in COVID-19 patients. DMRs (N=19) associated with the GRAM-score were identified in COVID-19 patients (N=100). DMRs were ascertained as regions with at least 3 consecutive CpGs where >75% of the CpGs in the region had a FDR P-value <0.05 and all were either hyper-methylated or hypo-methylated. DNA methylation levels of the DMPs (N=145) residing in the DMRs were subjected to recursive feature elimination to identify CpGs that best distinguish GRAM-score risk. Shown is a hierarchical cluster using the DNA methylation data from the 77 DMPS, that are shown as a heatmap of the M-values. Low GRAM-score risk (grey) and high GRAM-score risk (black) are indicated. These results indicate that DNA methylation levels at these 77 DMPs may be useful as biomarkers of the severity of COVID-19 patients.

FIG. 7: DNA methylation of 8 CpGs are identified to best distinguish corticosteroids responders from non-responders. Shown is a hierarchical cluster using the DNA methylation data, and a heatmap of the M-values. Steroid response of 0 (red) and 1 (green) are indicated.

FIG. 8A: Diagram of data generation and analysis pipeline. See Example 2 for details.

FIG. 8B: Clinical characteristics of participants. To prevent DNA methylation changes caused by asymptomatic SARS-CoV-2 infection, samples were taken from healthy volunteers enrolled in 2017, who were not recalled. IQR is interquartile range. Raw SF-36: Short Form Health Survey involves 36 questions that are divided in 9 domains. Each domain has a maximal score of 100% based on the participants answers and thus the optimal score is 900.

FIG. 8C: Pie chart showing the distribution of DMRs to standard genomic features in percent. 5′UTR=5′ untranslated region. In keeping with the known role of DNA methylation in regulation of gene expression, a preponderance of DMRs is in gene promoter regions.

FIG. 8D: Circos plot shows the genomic distribution of differentially methylated regions (DMRs) across the human genome (outer ring). Each chromosome is shown as a different color. Relative chromosome size is denoted by the arc bar length (Inner rings). Hyper-methylated DMRs are shown in red and hypo-methylated regions are shown in blue. Sex chromosomes were omitted from the analysis. These results indicate that 71 DNA regions remain differentially methylated one year after hospital discharge in reference to a pre-pandemic healthy control cohort.

FIG. 8E: Bar graph of the top 10 gene ontological (GO) processes related to the SARS-CoV-2-associated differentially methylated genes that remain abnormal one year after hospital discharge ordered by statistical significance. The X-axis provides the number of SARS-CoV-2 DMR-associated genes that contribute to each GO term. Bar color indicates the FDR P-value by using a Fischer test. These results indicate that the observed DMRs occur in genes that participate in process such as response to virus, regulation of immune processes and others.

### DETAILED DESCRIPTION OF THE INVENTION

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The genomic positions described herein refer to genomic positions as provided in the UCSC hg19 human reference genome (Fujita P A, Rhead B, Zweig A S, Hinrichs A S, Karolchik D, Cline M S, Goldman M, Barber G P, Clawson H, Coelho A, Diekhans M, Dreszer T R, Giardine B M, Harte R A, Hillman-Jackson J, Hsu F, Kirkup V, Kuhn R M, Learned K, Li C H, Meyer L R, Pohl A, Raney B J, Rosenbloom K R, Smith K E, Haussler D, Kent W J. The UCSC Genome Browser database: update 2011. Nucleic Acids Res. 2011 January; 39(Database issue): D876-82. doi: 10.1093/nar/gkg963.) and positions in other genomes aligning thereto. Suitable alignment methods are known in the art. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The genomic positions of any CpG sites described herein refer to the position of the C in the CpG site in the original genome.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

“Amplification” is a special case of nucleic acid replication involving template specificity. It is to be contrasted with non-specific template replication (e.g., replication that is template-dependent but not dependent on a specific template). Template specificity is here distinguished from fidelity of replication (e.g., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Amplification of nucleic acids generally refers to the production of multiple copies of a polynucleotide, or a portion of the polynucleotide, typically starting from a small amount of the polynucleotide (e.g., a single polynucleotide molecule, 10 to 100 copies of a polynucleotide molecule, which may or may not be exactly the same), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), Hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10: 413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.”

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-beta replicase, MDV-1 RNA is the specific template for the replicase (Kacian et al., Proc. Natl. Acad. Sci. USA, 69:3038 [1972]). Other nucleic acid will not be replicated by this amplification enzyme. Similarly, in the case of T7 RNA polymerase, this amplification enzyme has a stringent specificity for its own promoters (Chamberlin et al, Nature, 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics 4:560). Finally, thermostable template-dependent DNA polymerases (e.g., Taq and Pfu DNA polymerases), by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and US 2009/0253142, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Barnay Proc. Natl. Acad. Sci USA 88, 189-93 (1991)); and sandwich hybridization methods (e.g., U.S. Pat. No. 5,288,609, herein incorporated by reference in its entirety).

The term “amplifiable nucleic acid” refers to a nucleic acid that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as a DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated. With respect to a single CpG locus at a single pair of sister chromosomes in a single cell, a CpG may be 100% methylated (both Cs are methylated, 50% methylated (one C is methylated and the other is not), or 0% methylated (neither C of the paired sister chromosomes is methylated at the specific CpG locus). Accordingly, methylation of all sister chromosomes in a population of cells at a specific CpG may be 0-100% methylated depending on the relative proportion of cells that are 100%, 50% or 0% methylated at a specific CpG locus in a mixture of cells in a sample.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such a change in the nucleic acid molecule's nucleotide sequence can result in a nucleic acid molecule in which each methylated nucleotide is modified to a different nucleotide. Such a change in the nucleic acid nucleotide sequence can result in a nucleic acid molecule in which each unmethylated nucleotide is modified to a different nucleotide. Such a change in the nucleic acid nucleotide sequence can result in a nucleic acid molecule in which each of a selected nucleotide which is unmethylated (e.g., each unmethylated cytosine) is modified to a different nucleotide. Use of such a reagent to change the nucleic acid nucleotide sequence can result in a nucleic acid molecule in which each nucleotide that is a methylated nucleotide (e.g., each methylated cytosine) is modified to a different nucleotide. As used herein, use of a reagent that modifies a selected nucleotide refers to a reagent that modifies one nucleotide of the four typically occurring nucleotides in a nucleic acid molecule (C, G, T, and A for DNA and C, G, U, and A for RNA), such that the reagent modifies the one nucleotide without modifying the other three nucleotides. In one exemplary embodiment, such a reagent modifies an unmethylated selected nucleotide to produce a different nucleotide. In another exemplary embodiment, such a reagent can deaminate unmethylated cytosine nucleotides. An exemplary reagent is bisulfite.

As used herein, the term “bisulfite reagent” refers to a reagent comprising in some embodiments bisulfite, disulfite, hydrogen sulfite, or combinations thereof to distinguish between methylated and unmethylated cytidines, e.g., in CpG dinucleotide sequences.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al. (1997) Cancer Research 57: 594-599.

The term “MethyLight™” refers to the art-recognized fluorescence-based real-time PCR technique described by Eads et al. (1999) Cancer Res. 59: 2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25: 2529-2531.

The term “MSP” (Methylation-specific PCR) refers to the art-recognized methylation assay described by Herman et al. (1996) Proc. Natl. Acad. Sci. USA 93: 9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25: 2532-2534.

The term “MCA” (Methylated CpG Island Amplification) refers to the methylation assay described by Toyota et al. (1999) Cancer Res. 59: 2307-12, and in WO 00/26401A1.

The term “Infinium HD Methylation Assay” refers to the methylation assay described by Pidsley et al. (2016) Genome Biol. 17:208.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The terms “methylation-specific restriction enzyme” or “methylation-sensitive restriction enzyme” refers to an enzyme that selectively digests a nucleic acid dependent on the methylation state of its recognition site. In the case of a restriction enzyme that specifically cuts if the recognition site is not methylated or is hemimethylated, the cut will not take place or will take place with a significantly reduced efficiency if the recognition site is methylated. In the case of a restriction enzyme that specifically cuts if the recognition site is methylated, the cut will not take place or will take place with a significantly reduced efficiency if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, a “different nucleotide” refers to a nucleotide that is chemically different from a selected nucleotide, typically such that the different nucleotide has Watson-Crick base-pairing properties that differ from the selected nucleotide, whereby the typically occurring nucleotide that is complementary to the selected nucleotide is not the same as the typically occurring nucleotide that is complementary to the different nucleotide. For example, when C is the selected nucleotide, U or T can be the different nucleotide, which is exemplified by the complementarity of C to G and the complementarity of U or T to A. As used herein, a nucleotide that is complementary to the selected nucleotide or that is complementary to the different nucleotide refers to a nucleotide that base-pairs, under high stringency conditions, with the selected nucleotide or different nucleotide with higher affinity than the complementary nucleotide's base-paring with three of the four typically occurring nucleotides. An example of complementarity is Watson-Crick base pairing in DNA (e.g., A-T and C-G) and RNA (e.g., A-U and C-G). Thus, for example, G base-pairs, under high stringency conditions, with higher affinity to C than G base-pairs to G, A, or T and, therefore, when C is the selected nucleotide, G is a nucleotide complementary to the selected nucleotide.

As used herein, “health condition” refers to any one or more of respiratory disease infection, SARS-CoV-2 infection, a likelihood of developing critical illness from respiratory disease infection, a likelihood of developing critical illness from SARS-CoV-2 infection, a likelihood of dying from respiratory disease infection, a likelihood of dying from SARS-CoV-2 infection, a likelihood of responding to treatment (e.g., with steroids such as corticosteroids) of respiratory disease infection, a likelihood of responding to treatment (e.g., with steroids such as corticosteroids) of SARS-CoV-2 infection, or an absence of any of the foregoing. Any condition described herein can comprise one or more of the foregoing health conditions.

“Critical illness” as used herein refers at least to one or more of admission to an intensive care unit, invasive ventilation, and death.

“COVID-19” refers to SARS-CoV-2 infection.

As used herein, a “diagnostic” test application includes the detection or identification of a condition of a subject, determining the likelihood that a subject will contract a given condition, determining the likelihood that a subject with a condition will respond to therapy, determining the prognosis of a subject with a condition (or its likely progression or regression), and determining the effect of a treatment on a subject with condition.

The term “marker”, as used herein, refers to a substance (e.g., a nucleic acid or a region of a nucleic acid) that is able to indicate the presence of a health condition, e.g., based its methylation state.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, by be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it is meant to include a specimen or culture obtained from any source, as well as biological and environmental samples. Biological samples may be obtained from plants or animals (including humans) and encompass fluids, solids, tissues, and gases. In some embodiments, the sample is a blood sample. Environmental samples include environmental material such as surface matter, soil, water, and industrial samples. These examples are not to be construed as limiting the sample types applicable to the present invention.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In particular aspects, the present technology provides compositions and methods for diagnosing, predicting severity of, and/or predicting treatment response to respiratory virus infection, such as SARS-CoV-2 infection. The methods comprise determining the methylation status of at least one methylation marker in a biological sample isolated from a subject (e.g., blood sample), wherein a change in the methylation state of the marker is indicative of one or more health conditions. Particular embodiments relate to markers comprising one or more DMRs and/or DMPs in one or more of Tables 3, 6, 13, 14, 15, 16, 17A and 17B that are used for detecting the one or more health conditions.

Some embodiments of the technology are based upon the analysis of the CpG methylation status of at least one marker, region of a marker, or base of a marker comprising one or more DMRs and/or DMPs in one or more of Tables 3, 6, 13, 14, 15, 16, 17A and 17B.

In some embodiments, the present technology provides for the use of the bisulfite technique in combination with one or more methylation assays to determine the methylation status of CpG dinucleotide sequences within at least one marker comprising one or more DMRs and/or DMPs in one or more of Tables 3, 6, 13, 14, 15, 16, 17A and 17B. Genomic CpG dinucleotides can be methylated or unmethylated (alternatively known as up- and down-methylated respectively). However, the methods of the present invention are suitable for the analysis of biological samples of a heterogeneous nature. Accordingly, when analyzing the methylation status of a CpG position within such a sample one may use a quantitative assay for determining the level (e.g., percent, fraction, ratio, proportion, or degree) of methylation at a particular CpG position.

In some embodiments, the technology relates to assessing the methylation state of combinations of markers comprising more than one DMR and/or DMP in one or more of Tables 3, 6, 13, 14, 15, 16, 17A and 17B. In some embodiments, assessing the methylation state of more than one marker increases the specificity and/or sensitivity of detecting the health condition in a subject.

The most frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil forms base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98) or methylation-specific PCR (MSP) as is disclosed, e.g., in U.S. Pat. No. 5,786,146.

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then either assaying the product by sequencing (Olek & Walter (1997) Nat. Genet. 17: 275-6) or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures are known in the art and can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing or microarray of bisulfite-treated nucleic acid, PCR (for sequence-specific amplification), Southern blot analysis, and use of methylation-sensitive restriction enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

In some embodiments, assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, DMR, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase.

MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, DMR, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, DMR, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can by used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, DMR, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, DMR, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QUARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes cleavase to release the flap sequence by cutting between the detection probe and the flap sequence. The flap sequence is complementary to a nonhairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophore per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199; U.S. patent application Ser. Nos. 12/946,737, 12/946,745, 12/946,752, and 61/548,639.

In some embodiments, an array such as the Infinium HD methylation assay is used to evaluate methylation state. After bisulfite treatment, the samples are denatured and neutralized to prepare them for amplification. The denatured DNA is isothermally amplified in an overnight step. The whole-genome amplification uniformly increases the amount of the DNA sample by several thousand-folds without significant amplification bias. A controlled enzymatic process fragments the amplified product. The process uses endpoint fragmentation to prevent overfragmentation. After precipitation and resuspension, the fragmented DNA is dispensed onto BeadChips. The BeadChips are incubated in the Illumina Hybridization Oven to hybridize the samples onto the BeadChips. Twelve samples are applied to each BeadChip, which keeps them separate with an IntelliHyb seal. The prepared BeadChip is incubated overnight in the Illumina Hybridization Oven. The amplified and fragmented DNA samples anneal to locus-specific 50mers (covalently linked to 1 of over 500,000 bead types) during hybridization. Two bead types correspond to each CpG locus for Infinium I assays: one bead type corresponds to methylated (C), another bead type to unmethylated (T) state of the CpG site. One bead type corresponds to each CpG locus for Infinium II assays. Then the unhybridized and nonspecifically hybridized DNA is washed away and the BeadChip is prepared for staining and extension in capillary flow-through chambers. Single-base extension of the oligos occurs on the BeadChip, using the captured DNA as a template, which incorporates detectable labels on the BeadChip and determines the methylation level of the query CpG sites. The Illumina HiScan or iScan System scans the BeadChip, using a laser to excite the fluorophore of the single-base extension product on the beads. The scanner records high-resolution images of the light emitted from the fluorophores.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715, which is incorporated by reference in its entirety). It is preferred that the bisulfite treatment is conducted in the presence of denaturing solvents such as but not limited to n-alkylenglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8-tetramethylchromane 2-carboxylic acid or trihydroxybenzone acid and derivates thereof, e.g., Gallic acid (see: PCT/EP2004/011715, which is incorporated by reference in its entirety). The bisulfite conversion is preferably carried out at a reaction temperature between 30° C. and 70° C., whereby the temperature is increased to over 85° C. for short times during the reaction (see: PCT/EP2004/011715, which is incorporated by reference in its entirety). The bisulfite treated DNA is preferably purified prior to the quantification. This may be conducted by any means known in the art, such as but not limited to ultrafiltration, e.g., by means of Microcon™ columns (manufactured by Millipore™). The purification is carried out according to a modified manufacturer's protocol (see, e.g., PCT/EP2004/011715, which is incorporated by reference in its entirety).

In some embodiments, fragments of the treated DNA are amplified using sets of primer oligonucleotides according to the present invention and an amplification enzyme. The amplification of several DNA segments can be carried out simultaneously in one and the same reaction vessel. Typically, the amplification is carried out using a polymerase chain reaction (PCR). Amplicons are typically 100 to 2000 base pairs in length.

In another embodiment of the method, the methylation status of CpG positions within or near a marker comprising any one or more DMRs and/or DMPs in one or more of Tables 3, 6, 13, 14, 15, 16, 17A and 17B may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

Genomic DNA may be isolated by any means, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types are suitable for use in the present method, e.g., cell lines, histological slides, biopsies, paraffin-embedded tissue, body fluids, stool, prostate tissue, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a blood sample using direct gene capture.

The genomic DNA sample is then treated with at least one reagent, or series of reagents, that distinguishes between methylated and non-methylated CpG dinucleotides within at least one marker comprising one or more DMRs and/or DMPs in one or more of Tables 3, 6, 13, 14, 15, 16, 17A and 17B.

In some embodiments, the reagent converts cytosine bases which are unmethylated at the 5′-position to uracil, thymine, or another base which is dissimilar to cytosine in terms of hybridization behavior. However in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one gene, genomic sequence, or nucleotide from a marker comprising one or more DMRs and/or DMPs in one or more of Tables 3, 6, 13, 14, 15, 16, 17A and 17B. The method of analysis may be selected from those known in the art, including those listed herein, e.g., QuARTS and MSP as described herein.

Aberrant methylation of a marker comprising one or more DMRs and/or DMPs in one or more of Tables 3, 6, 13, 14, 15, 16, 17A and 17B as shown in the examples is associated with one or more health conditions described herein.

In some embodiments, the technology relates to a method for treating a patient (e.g., a patient with one or more health conditions as described herein), the method comprising determining the methylation state of any one or more DMRs or DMPs as provided herein and administering a treatment to the patient based on the results of determining the methylation state. The treatment may be administration of a pharmaceutical compound, a vaccine, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, the methods comprise determining the methylation state of one or more DMRs and/or DMPs in one or more of Tables 16 and 17 in DNA obtained from a subject with a respiratory disease such as SARS-CoV-2, determining a likelihood of response to therapy with a steroid such as a corticosteroid, and treating a subject with a steroid such as a corticosteroid if the subject has a likelihood of responding to therapy therewith.

In some embodiments of the technology, a method for diagnosing a respiratory infection, such as SARS-CoV-2 infection, in a subject is provided. The terms “diagnosing” and “diagnosis” as used herein refer to methods by which the skilled artisan can estimate and even determine whether or not a subject is suffering from a given condition or may develop a given condition in the future. The skilled artisan often makes a diagnosis on the basis of one or more diagnostic indicators, such as for example a biomarker (e.g., a DMR and/or DMP as disclosed herein), the methylation state of which is indicative of the presence, severity, or absence of the condition.

Along with diagnosis, clinical respiratory virus infection prognosis relates to determining the projected clinical outcomes of the respiratory virus infection and the likelihood of response to therapy to plan the most effective therapy. If a more accurate prognosis can be made or even a potential risk for developing severe outcomes can be assessed, appropriate therapy, and in some instances less severe therapy for the patient can be chosen. Assessment (e.g., determining methylation state) of respiratory virus infection biomarkers is useful to separate subjects with good prognosis and/or low risk of developing severe outcomes who will need no therapy or limited therapy from those more likely to develop severe outcomes who might benefit from more intensive treatments.

As such, “making a diagnosis” or “diagnosing”, as used herein, is further inclusive of determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMRs and/or DMPs) disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of respiratory virus infection, and/or monitor the efficacy of appropriate therapies directed against the infection. In such an embodiment for example, one might expect to see a change in the methylation state of one or more biomarkers (e.g., DMRs and/or DMPs) disclosed herein (and potentially one or more additional biomarker(s), if monitored) in a biological sample over time during the course of an effective therapy.

The presently disclosed subject matter further provides in some embodiments a method for determining whether to initiate or continue treatment of a respiratory virus infection in a subject. In some embodiments, the method comprises providing a series of biological samples over a longitudinal or serial time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict risk of developing severe symptoms, predict clinical outcome, determine whether to initiate or continue treatment or therapy, and whether a current therapy is effectively treating the respiratory virus infection. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with respiratory virus infection risk, prognosis, determining treatment efficacy, and/or progression of the respiratory virus infection in the subject.

In preferred embodiments, the methods and compositions of the invention are for treatment or diagnosis of disease at an early stage, for example, before symptoms of the disease appear or before severe clinical outcomes begin to emerge. In some embodiments, the methods and compositions of the invention are for treatment or diagnosis of disease at a clinical stage.

As noted, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of respiratory virus infection, or a given prognosis. Likewise, a decrease in the marker from the initial time to the second time can be indicative of a particular type or severity of respiratory virus infection, or a given prognosis. Furthermore, the degree of change of one or more markers can be related to the severity of the respiratory virus infection and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR and/or DMP). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMRs and/or DMPs), the chance of a given outcome (e.g., suffering from severe clinical outcomes) may be very low.

In some embodiments, a statistical analysis associates a prognostic indicator with a predisposition to an adverse outcome. For example, in some embodiments, a methylation state different from that in a normal control sample obtained from a patient who does not have a particular adverse outcome can signal that a subject is more likely to suffer from a particular adverse outcome than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein (e.g., a DMR and/or DMP) can be established, and the degree of change in the methylation state of the biomarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal samples, as well as for “at-risk” levels of the one or more biomarkers in samples taken from donors with a particular health condition. In certain embodiments of the method, a subject is identified as having a particular health condition upon identifying an aberrant methylation state of one or more DMRs and/or DMPs provided herein in a biological sample obtained from the subject. In other embodiments of the method, the detection of an aberrant methylation state of one or more of such biomarkers in a biological sample obtained from the subject results in the subject being identified as having a particular health condition.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having a particular health condition if, when compared to a control methylation state, there is a measurable difference in the methylation state of at least one biomarker in the sample. Conversely, when no change in methylation state is identified in the biological sample, the subject can be identified as not having the health condition, not being at risk for the health condition, or as having a low risk of the health condition. In this regard, subjects having the health condition or risk thereof can be differentiated from subjects having low to substantially no health condition or risk thereof. Those subjects having a risk of developing a particular health condition can be placed on a more intensive and/or regular screening schedule or treatment. On the other hand, those subjects having low to substantially no risk may avoid being subjected to more intensive and/or regular screening schedule or treatment.

Depending on the embodiment of the method of the present technology, detecting a change in methylation state of the one or more biomarkers can be a qualitative determination or it can be a quantitative determination. As such, the step of diagnosing a subject as having, or at risk of developing a particular health condition indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specifically state or range of state. As such, the predetermined methylation state can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. As such, the present technology provides for the diagnosis of mammals such as humans.

Madrid et al. 2021 (Balnis J, Madrid A, Hogan K J, Drake L A, Chieng H C, Tiwari A, Vincent C E, Chopra A, Vincent P A, Robek M D, Singer H A, Alisch R S, Jaitovich A. Blood DNA methylation and COVID-19 outcomes. Clin Epigenetics. 2021 May 25; 13(1):118) is incorporated herein by reference in its entirety.

The elements and method steps described herein can be used in any combination whether explicitly described or not, unless otherwise specified or clearly implied to the contrary by the context in which the referenced combination is made.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

As used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. The meaning of “in” includes “in” and “on.”

### EXAMPLES

### Electronic Tables

Tables 2, 3, 4A, 4B, 5A, 5B, 6, 7, 8, 11, 12A, 12B, 16, and 19 have been submitted as ASCII text files via EFS-Web and are incorporated by reference.

LENGTHY TABLES
The patent application contains a lengthy table section. A copy of the table is available in electronic form from the USPTO web site (https://seqdata.uspto.gov/?pageRequest=docDetail&DocID=US20220364187A1). An electronic copy of the table will also be available from the USPTO upon request and payment of the fee set forth in 37 CFR 1.19(b)(3).

The ASCII text file of Table 2 is named “Table_2.txt,” was created on Apr. 26, 2021, and is 58,298 bytes in size.

The ASCII text file of Table 3 is named “Table_3.txt,” was created on Apr. 26, 2021, and is 327,236 bytes in size.

The ASCII text file of Table 4A is named “Table_4A.txt,” was created on Apr. 26, 2021, and is 102,102 bytes in size.

The ASCII text file of Table 4B is named “Table_4B.txt,” was created on Apr. 26, 2021, and is 205,216 bytes in size.

The ASCII text file of Table 5A is named “Table_5A.txt,” was created on Apr. 26, 2021, and is 167,346 bytes in size.

The ASCII text file of Table 5B is named “Table_5B.txt,” was created on Apr. 26, 2021, and is 294,030 bytes in size.

The ASCII text file of Table 6 is named “Table_6.txt,” was created on Apr. 26, 2021, and is 41,565 bytes in size.

The ASCII text file of Table 7 is named “Table_7.txt,” was created on Apr. 26, 2021, and is 25,132 bytes in size.

The ASCII text file of Table 8 is named “Table_8.txt,” was created on Apr. 26, 2021, and is 19,593 bytes in size.

The ASCII text file of Table 11 is named “Table_11.txt,” was created on Apr. 26, 2021, and is 2,170 bytes in size.

The ASCII text file of Table 12A is named “Table_12A.txt,” was created on Apr. 26, 2021, and is 23,014 bytes in size.

The ASCII text file of Table 12B is named “Table_12B.txt,” was created on Apr. 26, 2021, and is 28,943 bytes in size.

The ASCII text file of Table 16 is named “Table_16.txt,” was created on Apr. 26, 2021, and is 25,872 bytes in size.

The ASCII text file of Table 19 is named “Table_19.txt,” was created on Apr. 26, 2021, and is 39,449 bytes in size.

The ASCII text file of Table 21 is named “Table_21.txt,” was created on May 16, 2022, and is 704,568 bytes in size.

### Example 1

### Summary

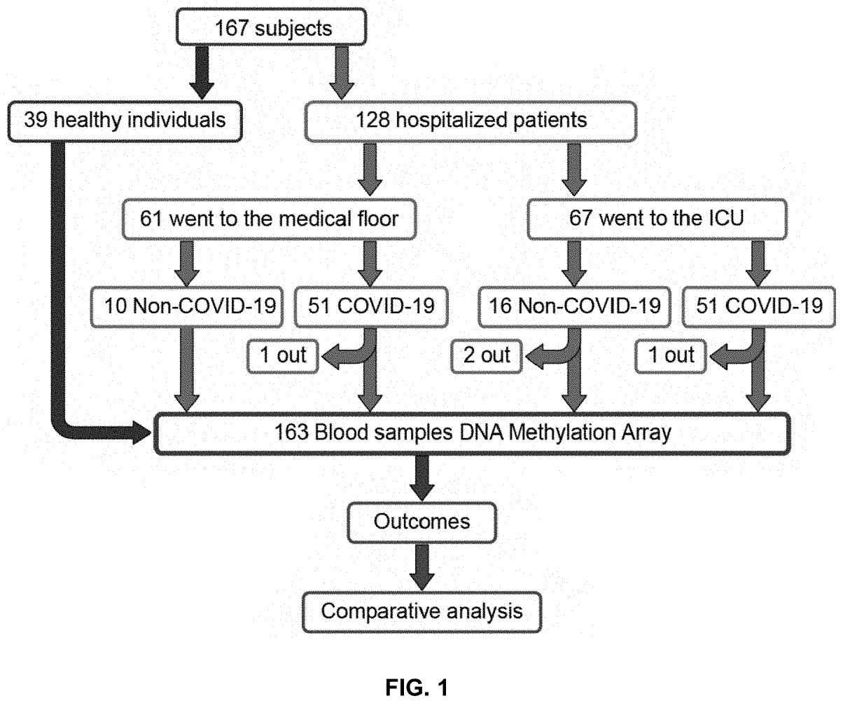

We conducted a prospective cohort study involving 124 consecutive patients with and without COVID-19 diagnosis who were admitted to Albany Medical Center in Albany, N.Y. Thirty-nine healthy patient samples collected before the COVID-19 pandemic characterized with an identical epigenotyping platform provided reference methylomes (FIG. 1). We hypothesized that: 1) DNA methylation regions would differ in patients with COVID-19 diagnosis in comparison with pre-pandemic healthy control individuals; 2) DNA methylation regions would differ in patients with COVID-19 diagnosis in comparison with patients with respiratory illness of similar magnitude not caused by COVID-19; and 3) COVID-19 severity reflected by clinically validated outcome measures⁴⁰, would be associated with distinct patterns of DNA methylation in blood.

### Results

Sample cohort and experimental design: From 6 Apr. 2020 through 1 May 2020, we collected blood samples from 128 adult patients admitted to the Albany Medical Center in Albany, N.Y. for moderate to severe respiratory failure presumably related to infection with SARS-CoV-2 (FIG. 1). In addition to acquisition of various clinical data (Table 1), a 10 ml blood sample was obtained at the time of enrollment. Patients who later tested positive (N=102) and negative (N=26) for SARS-CoV-2 infection were assigned to the COVID-19 and non-COVID-19 groups, respectively (see Methods for enrollment details). Females comprised 37.3% and 50.0% of the COVID-19 and non-COVID-19 patients, respectively. The average age of patients was similar: 60.5 (50.5-74.8) and 62 (50-74) years in the COVID-19 group (females and males, respectively; P-value=0.28) compared the non-COVID-19 patients: 59.5 (49-75) and 68.2 (63-82) years, (females and males respectively; P-value=0.09). The average number of days hospitalized before study enrollment was 3 and 1 for the COVID-19 and non-COVID-19 patients, respectively (Table 1). The COVID-19 group was more racially diverse than the non-COVID-19 group, with white individuals comprising 46% of the total (vs. 80% of the non-COVID-19 control group), consistent with racial and ethnic health disparities of COVID-19 reported by others⁴¹. To identify DNA CpG methylation changes associated with COVID-19, we compared DNA methylation data from COVID-19 patients (N=102) to DNA methylation data from a previously published study²¹ that profiled DNA methylation from whole blood of healthy participants (N=39) that was collected at least 3 years before the COVID-19 outbreak. An identical epigenotyping platform and facility (Genuity Science, Inc. Boston, Mass.) was used to obtain the methylation data. To test whether COVID-19 severity correlates with patterns of differential DNA methylation in blood, we used the COVID-19 specific GRAM risk score⁴⁰ and patient mortality. Other clinical data included: Acute Physiologic Assessment and Chronic Health Evaluation (APACHE II) score, Sequential Organ Failure Assessment (SOFA) score⁴², SAPS II score, Charlson Comorbidity Index score⁴³, mechanical ventilation physiological parameters, need for admission to intensive care, and C-reactive protein (CRP), D-dimer, ferritin, lactate, procalcitonin, fibrinogen, and other levels (Table 1). APACHE II, SOFA and SAPS II severity scores assigned to patients in intensive care, exhibited similar distributions between the groups (Table 1). In keeping with previous reports, males predominated in the group requiring intensive care (66 vs. 33%) and mechanical ventilation (46.9 vs. 34.2%, see Table 2).

TABLE 1
Demographics and baseline characteristics of COVID-19
in ICU and non-ICU setting and healthy controls.
COVID-19

| | Total | | non-ICU | | ICU | |
|---|---|---|---|---|---|---|
| Variables | n = 102 | | n = 51 | | n = 51 | |
| Days Admitted Pre- | 3.37 | (1-5) | 2.78 | (1-3) | 3.96 | (1-6) |
| Enrollment (IQR) | | | | | | |
| Sex - n (%) | | | | | | |
| Male | 64 | (62.7%) | 30 | (58.8%) | 34 | (66.7%) |
| Female | 38 | (37.3%) | 21 | (41.2%) | 17 | (33.3%) |
| Age-year | | | | | | |
| Mean (IQR) | 61.3 | (50.0-74.3) | 59.7 | (49.0-80.0) | 62.9 | (55.0-73.0) |
| Ethnicity - n (%) | | | | | | |
| White | 46 | (45.1%) | 28 | (54.9%) | 18 | (35.3%) |
| Black | 11 | (10.8%) | 5 | (9.8%) | 6 | (11.8%) |
| Asian | 2 | (1.9%) | 0 | (0%) | 2 | (3.9%) |
| Hispanic | 21 | (20.6%) | 7 | (13.7%) | 14 | (27.5%) |
| Other | 22 | (21.6%) | 11 | (21.6%) | 11 | (21.6%) |
| BMI, kg/m2 Mean (IQR) | 30.39 | (25.30-32.24) | 29.84 | (26.09-32.37) | 30.92 | (24.50-32.05) |
| Severity Indexes (IQR) | | | | | | |
| Charlson comorbidity index | 3.3 | (1-5) | 3.16 | (1-5) | 3.49 | (2-5) |
| APACHEII | N/A | | N/A | | 21.6 | (15-27) |
| SOFA | N/A | | N/A | | 8.2 | (6-11) |
| SAPSII | N/A | | N/A | | 51.8 | (45-62) |
| Biomarkers (IQR) | | | | | | |
| Ferritin (ng/mL) | 938.9 | (301.8-1203.8) | 782.6 | (206.0-934.5) | 1076.9 | (378.0-1294.0) |
| C-Reactive protein (mg/L) | 140.9 | (52.0-204.3) | 120.6 | (44.7-155.0) | 158.9 | (61.7-248.3) |
| D-dimer (mg/L FEU) | 11.7 | (1.0-12.8) | 2.3 | (0.6-1.73) | 18.6 | (1.7-21.6) |
| Procalcitonin (ng/mL) | 3.2 | (0.2-1.8) | 1.7 | (0.2-1.0) | 4.4 | (0.3-2.3) |
| Lactate (mmol/L) | 1.2 | (0.9-1.5) | 1.2 | (0.9-1.4) | 1.3 | (0.9-1.5) |
| Fibrinogen (mg/dL) | 543.6 | (413.0-667.0) | 559.3 | (420.0-703.0) | 531.7 | (391.5-663.0) |
| Albumin (mg/L) | 2.9 | (2.6-3.3) | 3.2 | (2.9-3.5) | 2.7 | (2.4-2.9) |
| Hemogram (IQR) | | | | | | |
| White blood cells (K/uL) | 10.8 | (6.1-12.5) | 7.1 | (4.9-8.5) | 14.4 | (8.4-15.4) |
| Hemoglobin (g/dL) | 11.2 | (9.7-12.6) | 11.6 | (10.2-13.0) | 10.7 | (9.4-12.1) |
| Mean corpuscular volume (fL) | 87.1 | (84.5-93.7) | 88.0 | (85.6-94.2) | 86.2 | (82.5-93.0) |
| Platelet (K/uL) | 266.0 | (192.5-320.5) | 269.2 | (209.0-334) | 262.8 | (187.0-317.0) |
| Neutrophils (%) | 76.2 | (68.5-86.0) | 69.7 | (61.0-82.0) | 82.8 | (80.0-90.0) |
| Lymphocytes (%) | 13.8 | (5.0-18.5) | 19.4 | (9.0-26.0) | 8.3 | (4.0-11.0) |
| Monocytes (%) | 7.1 | (4.0-9.0) | 8.8 | (6.0-11.0) | 5.5 | (3.0-8.0) |
| Eosinophils (%) | 0.8 | (0.0-1.0) | 1.1 | (0.0-1.0) | 0.5 | (0.0-1.0) |
| Respiratory parameters | | | | | | |
| PaO2/FiO2 Ratio | N/A | | N/A | | 161.6 | (98-211) |
| Positive-end expiratory | N/A | | N/A | | 10.8 | (10-12) |
| pressure (cmH2O) | | | | | | |
| Inspiratory Plateau (cmH2O) | N/A | | N/A | | 22.8 | (19.7-25.3) |
| Treatment - n (%) | | | | | | |
| Renal Relacement Therapy | 12 | (11.8%) | 3 | (5.9%) | 9 | (17.6%) |
| Hydroxychloroquine | 87 | (85.3%) | 43 | (84.3%) | 44 | (86.3%) |
| Antibiotics | 98 | (96.1%) | 47 | (92.2%) | 51 | (100%) |
| Antiviral | 1 | (0.98%) | 0 | (0%) | 1 | (1.9%) |
| IL6- Antagoinist | 4 | (3.9%) | 1 | (1.9%) | 2 | (3.9%) |
| Convalescent Plasma | 26 | (25.5%) | 8 | (15.7%) | 18 | (35.3%) |
| Steroid | 46 | (45.1%) | 12 | (23.5%) | 34 | (66.7%) |
| Therapeutic Anticoagulation | 37 | (36.3%) | 2 | (3.9%) | 35 | (68.6%) |

| non-COVID-19 | | | |
|---|---|---|---|
| | Total | non-ICU | ICU |
| Variables | n = 26 | n = 10 | n = 16 |

| Days Admitted Pre- | 0.97 | (1-1) | 0.9 | (0.8-1) | 0.94 | (1-1) |
|---|---|---|---|---|---|---|
| Enrollment (IQR) | | | | | | |
| Sex - n (%) | | | | | | |
| Male | 13 | (50%) | 4 | (40%) | 9 | (56%) |
| Female | 13 | (50%) | 6 | (60%) | 7 | (44%) |
| Age-year | | | | | | |
| Mean (IQR) | 63.8 | (52.3-76.8) | 60.4 | (47.3-74.0) | 66 | (55.3-80.3) |
| Ethnicity - n (%) | | | | | | |
| White | 21 | (80.8%) | 8 | (80%) | 13 | (81.2%) |
| Black | 4 | (15.4%) | 2 | (20%) | 2 | (12.5%) |
| Asian | 0 | (0%) | 0 | (0%) | 0 | (0%) |
| Hispanic | 1 | (3.8%) | 0 | (0%) | 1 | (6.3%) |
| Other | 0 | (0%) | 0 | (0%) | 0 | (0%) |
| BMI, kg/m2 Mean (IQR) | 30.36 | (26.53-33.10) | 27.20 | (23.68-30.38) | 32.34 | (26.98-37.67) |
| Severity Indexes (IQR) | | | | | | |
| Charlson comorbidity index | 4.35 | (2-6) | 3.3 | (1-5) | 5 | (3-7) |
| APACHEII | N/A | | N/A | | 20.6 | (12-26) |
| SOFA | N/A | | N/A | | 8.6 | (3-11) |
| SAPSII | N/A | | N/A | | 47.6 | (33-65) |
| Biomarkers (IQR) | | | | | | |
| Ferritin (ng/mL) | 250.5 | (80.5-382.5) | 205.3 | (58.0-411.0) | 285.7 | (92.0-438.5) |
| C-Reactive protein (mg/L) | 73.8 | (20.0-110.2) | 34.7 | (8.9-56.8) | 99.8 | (37.8-175.2) |
| D-dimer (mg/L FEU) | 5.3 | (0.5-4.6) | 5.2 | (0.4-1.9) | 5.5 | (0.6-10.2) |
| Procalcitonin (ng/mL) | 2.1 | (0.2-0.7) | 2.2 | (0.1-3.4) | 2.1 | (0.3-1.21) |
| Lactate (mmol/L) | 2.1 | (0.9-2.5) | 1.2 | (0.8-1.5) | 2.53 | (0.9-3.4) |
| Fibrinogen (mg/dL) | 362.3 | (257.3-550.0) | 348.0 | (256.75-441.5) | 373 | (257.3-572.0) |
| Albumin (mg/L) | 3.4 | (2.9-3.8) | 3.8 | (3.4-4.1) | 3.19 | (2.6-3.8) |
| Hemogram (IQR) | | | | | | |
| White blood cells (K/uL) | 12.7 | (7.2-17.3) | 8.3 | (6.7-9.7) | 15.4 | (8.2-20.9) |
| Hemoglobin (g/dL) | 12.4 | (9.9-14.7) | 12.8 | (10.45-14.85) | 12.3 | (9.6-14.5) |
| Mean corpuscular volume (fL) | 92.3 | (88.6-95.4) | 91.2 | (87.2-94.6) | 93.0 | (89.4-97.8) |
| Platelet (K/uL) | 203.5 | (151.8-247.8) | 228.1 | (163.5-278.0) | 188.2 | (127.5-229.5) |
| Neutrophils (%) | 77.7 | (74.0-87.0) | 73.1 | (58.8-82.5) | 80.5 | (79.25-89.25) |
| Lymphocytes (%) | 12.7 | (6.0-18.0) | 16.9 | (7.0-26.0) | 10.1 | (4.3-10.8) |
| Monocytes (%) | 8.0 | (4.0-9.3) | 7.7 | (4.0-10.3) | 8.2 | (4.0-9.0) |
| Eosinophils (%) | 1.0 | (0.0-1.25) | 1.8 | (0.0-3.3) | 0.44 | (0.0-1.0) |
| Respiratory parameters | | | | | | |
| PaO2/FiO2 Ratio | N/A | | N/A | | 149.4 | (73-184) |
| Positive-end expiratory | N/A | | N/A | | 6.6 | (73-184) |
| pressure (cmH2O) | | | | | | |
| Inspiratory Plateau (cmH2O) | N/A | | N/A | | 23.9 | (19.8-28.8) |
| Treatment - n (%) | | | | | | |
| Renal Relacement Therapy | 3 | (11.5%) | 0 | (0%) | 3 | (18.8%) |
| Hydroxychloroquine | 0 | (0%) | 0 | (0%) | 0 | (0%) |
| Antibiotics | 16 | (61.5%) | 3 | (30.0%) | 13 | (81.3%) |
| Antiviral | 0 | (0%) | 0 | (0%) | 0 | (0%) |
| IL6- Antagoinist | 0 | (0%) | 0 | (0%) | 0 | (0%) |
| Convalescent Plasma | 0 | (0%) | 0 | (0%) | 0 | (0%) |
| Steroid | 4 | (15.4%) | 1 | (10.0%) | 3 | (18.8%) |
| Therapeutic Anticoagulation | 8 | (30.8%) | 1 | (10.0%) | 7 | (43.8%) |

Healthy

| | Total | |
|---|---|---|
| Variables | n = 39 | |
| Days Admitted Pre- | N/A | |
| Enrollment (IQR) | | |
| Sex - n (%) | | |
| Male | 18 | (46%) |
| Female | 21 | (54%) |
| Age-year | | |
| Mean (IQR) | 75.8 | (71.9-78.8) |
| Ethnicity - n (%) | | |
| White | 35 | (89.7%) |
| Black | 4 | (10.3%) |
| Asian | 0 | (0%) |
| Hispanic | 0 | (0%) |
| Other | 0 | (0%) |
| BMI, kg/m2 Mean (IQR) | 28.52 | (24.15-30.40) |
| Severity Indexes (IQR) | | |
| Charlson comorbidity index | N/A | |
| APACHEII | N/A | |
| SOFA | N/A | |
| SAPSII | N/A | |
| Biomarkers (IQR) | | |
| Ferritin (ng/mL) | N/A | |
| C-Reactive protein (mg/L) | N/A | |
| D-dimer (mg/L FEU) | N/A | |
| Procalcitonin (ng/mL) | N/A | |
| Lactate (mmol/L) | N/A | |
| Fibrinogen (mg/dL) | N/A | |
| Albumin (mg/L) | N/A | |
| Hemogram (IQR) | | |
| White blood cells (K/uL) | N/A | |
| Hemoglobin (g/dL) | N/A | |
| Mean corpuscular volume (fL) | N/A | |
| Platelet (K/uL) | N/A | |
| Neutrophils (%) | N/A | |
| Lymphocytes (%) | N/A | |
| Monocytes (%) | N/A | |
| Eosinophils (%) | N/A | |
| Respiratory parameters | | |
| PaO2/FiO2 Ratio | N/A | |
| Positive-end expiratory | N/A | |
| pressure (cmH2O) | | |
| Inspiratory Plateau (cmH2O) | N/A | |
| Treatment - n (%) | | |

| | |
|---|---|
| Renal Relacement Therapy | N/A |
| Hydroxychloroquine | N/A |
| Antibiotics | N/A |
| Antiviral | N/A |
| 1L6- Antagoinist | N/A |
| Convalescent Plasma | N/A |
| Steroid | N/A |
| Therapeutic Anticoagulation | N/A |

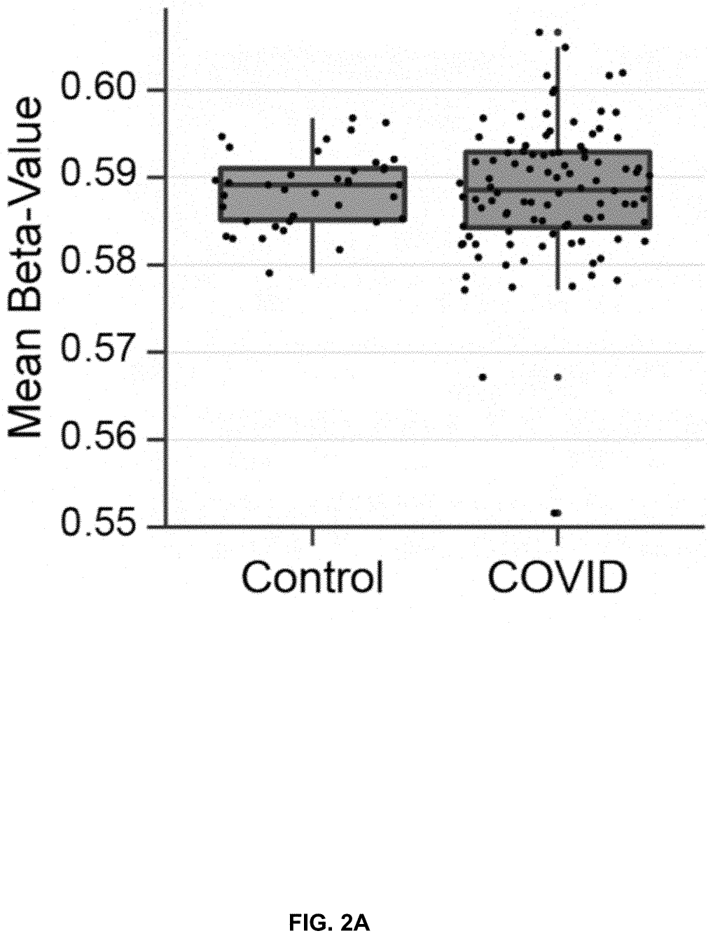

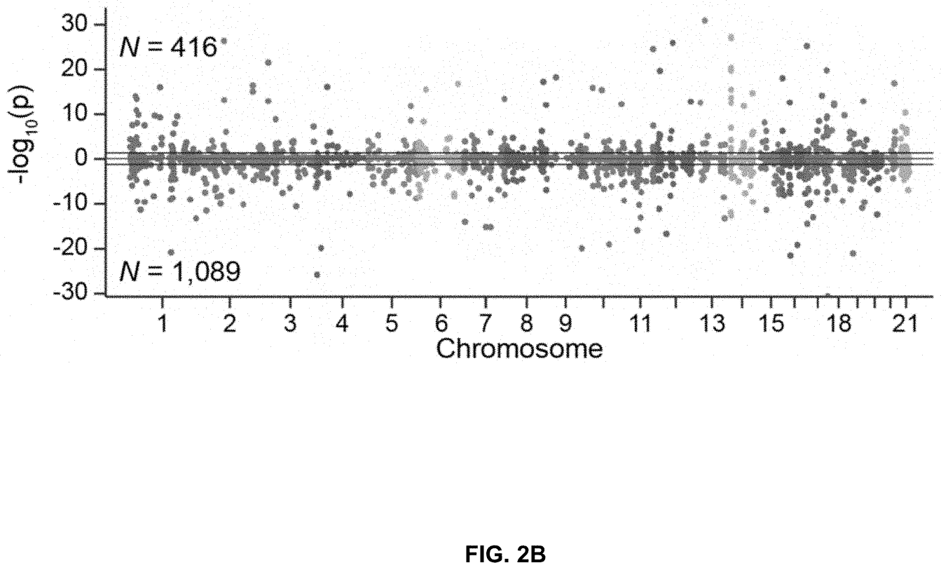

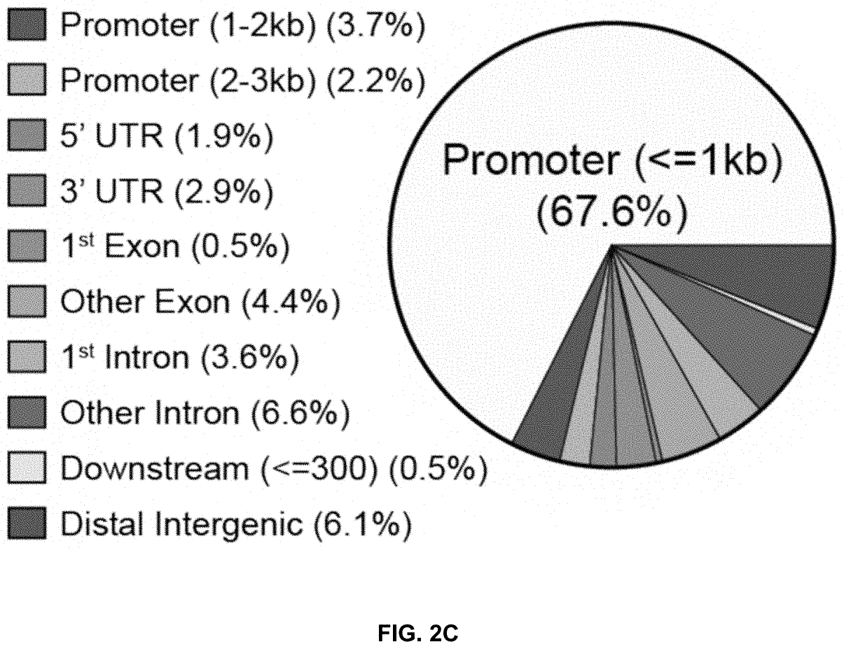

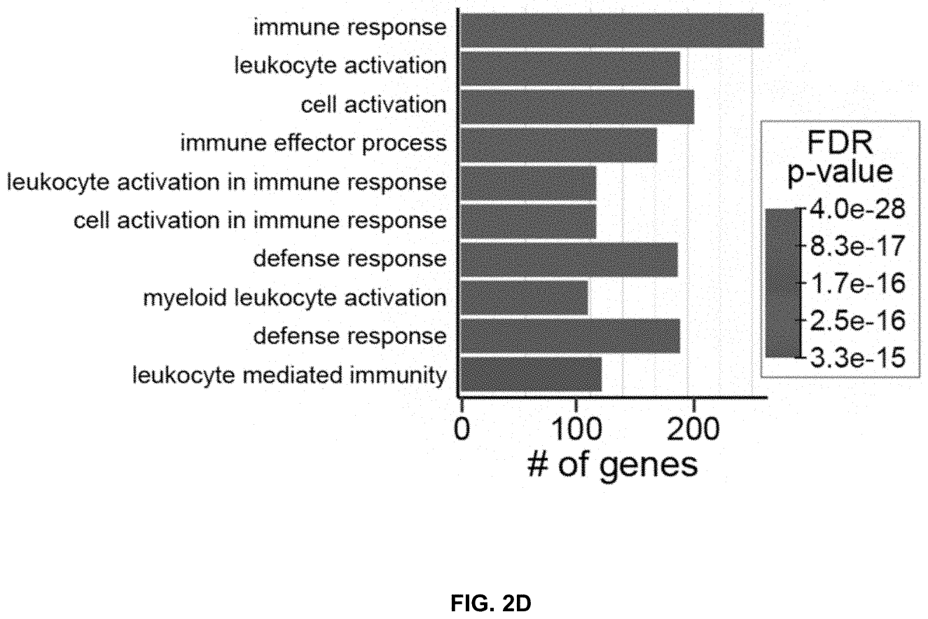

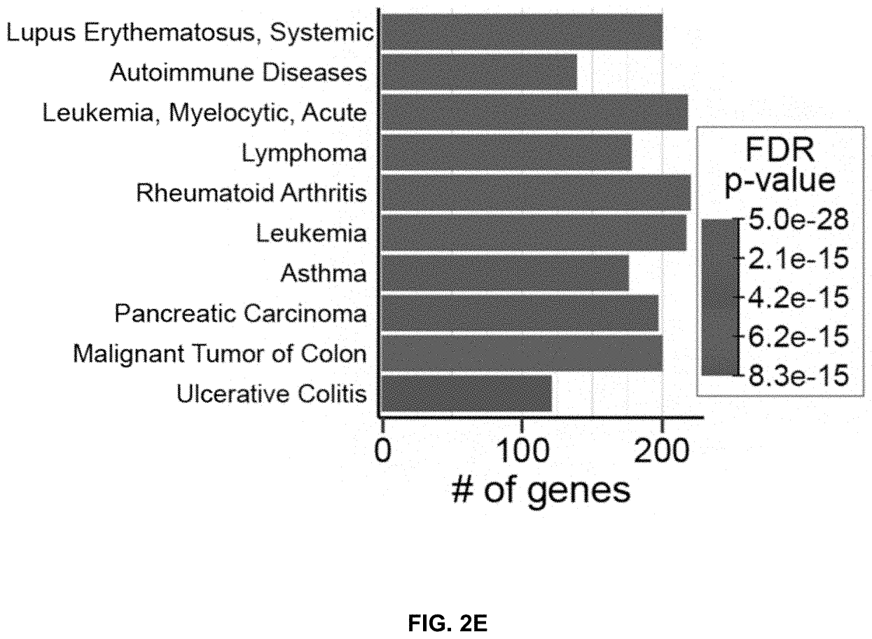

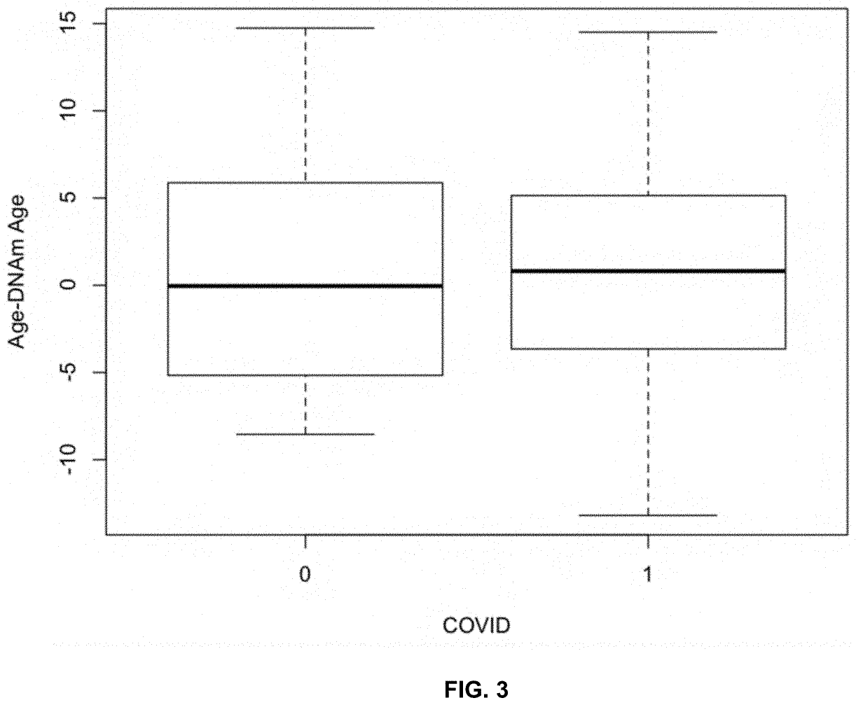

DNA methylation in blood is altered in COVID-19 patients: Average DNA methylation abundance across the entire genome did not significantly differ between COVID-19 patients (58.8%) and healthy pre-pandemic controls (58.7%), indicating that no global changes in methylation abundance are related to COVID-19 (FIG. 2A). To investigate locus-specific DNA methylation levels linked to COVID-19, methylome data were subjected to a linear regression model that accounted for batch effects, sex, and leukocyte proportions for downstream analyses⁴⁴. This approach detected 1,505 differentially methylated regions (DMRs) distributed across the entire genome comprising clusters of ≥5 CpGs (false discovery rate (FDR) P-value <0.05; FIG. 2B; Table 3). A total of 416 hyper-methylated and 1,089 hypo-methylated DMRs were distinguished, indicating that a majority of differentially methylated regions are hypo-methylated, as noted in a recent report of 7 COVID-19 positive patients¹⁰. A majority of DMRs (˜75%) reside within or near gene promoter regions, denoting a potential role in gene regulation¹⁶ (FIG. 2C). The 1,505 DMRs were annotated to 1,680 unique genes, indicating that several DMRs spanned two contiguous genes that harbor alterations in DNA methylation in the presence of SARS-CoV-2 infection. To test the relationships between the DMR-associated genes, we conducted a gene ontological analysis and found significant enrichments of immune-related terms, including immune responsivity, leukocyte activation, and defense responses, together with a diversity of recognized immune function genes (cytokines/chemokines and receptors (including IL-10, IL-1β, CXCR2/5/6), interferon-stimulated genes (IFIT3, ISG20), and signal transduction genes (TRAF2, ZAP70), (FDR P-value <0.05; FIG. 2D; Tables 4A and 4B). A disease ontological analysis of methylation regions that differ between COVID-19 patients and healthy pre-pandemic controls indicated significant associations of DMR-associated genes with autoimmune diseases, including systemic lupus erythematosus and rheumatoid arthritis (FDR P-value <0.05; FIG. 2E; Table 5A and 5B). We observed no difference in the gap between chronologic age and “epigenetic clock” age between COVID-19 patients and healthy pre-pandemic controls, suggesting that there is no difference between the two groups in predisposition and resilience to an acute infection known to have enhanced severity in the elderly^{45,46} (FIG. 3). These findings indicate that differential patterns of COVID-19 DNA methylation in blood occur in the promoter regions of immune-related genes.

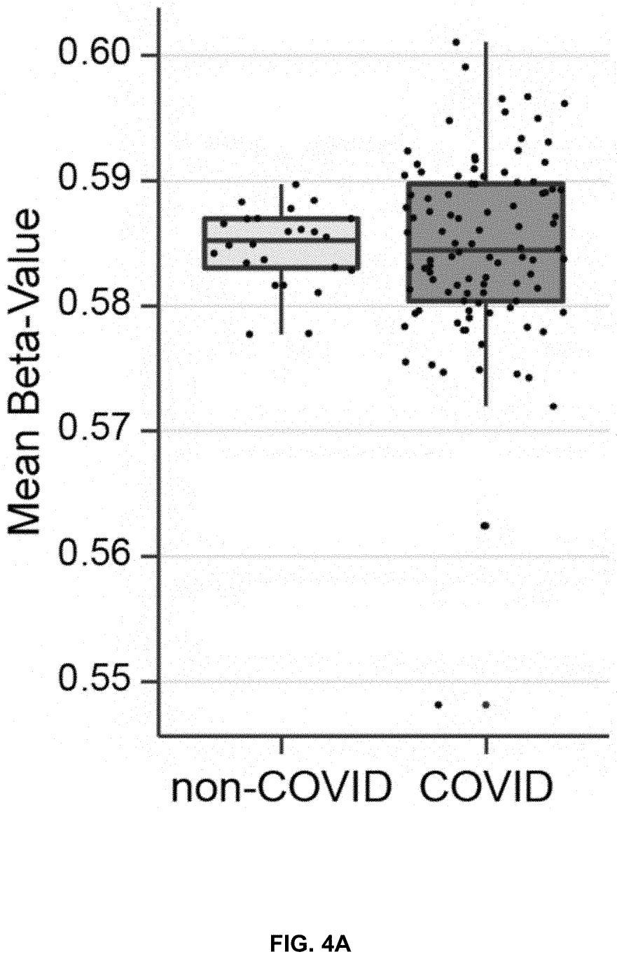

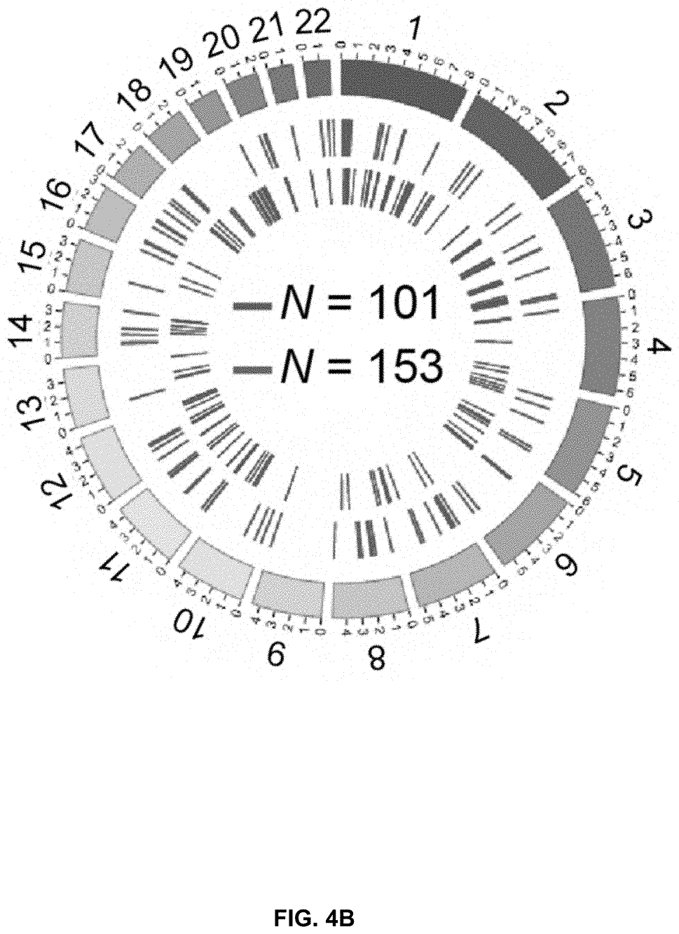

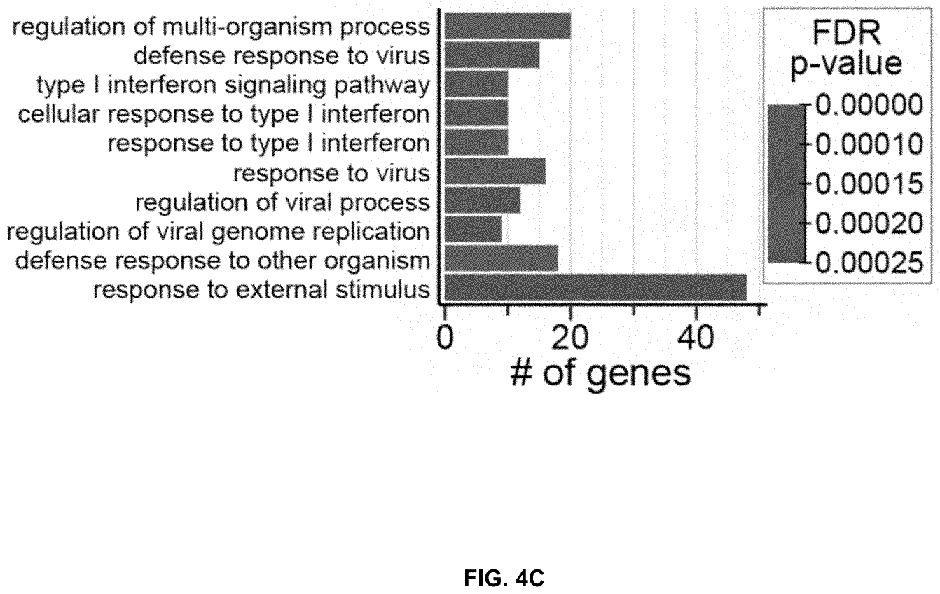

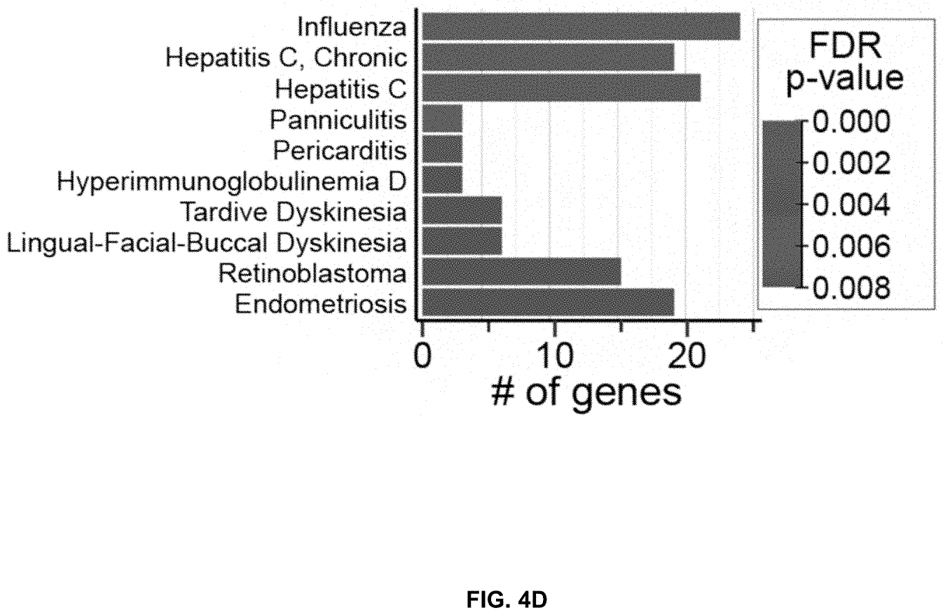

DNA methylation in blood is specific to SARS-CoV-2 infection: To identify DNA methylation profiles that distinguish concurrently enrolled respiratory patients with and without COVID-19, we analyzed data of 128 patients, with (N=102) and without (N=26) COVID-19 diagnosis collected concurrently at Albany Medical Center (FIG. 1 and Table 1). Four samples (two COVID-19 and two non-COVID-19 patients) were removed due to unreliable methylation values (FIG. 1) and 95,447 probes were removed leaving 770,412 for further analysis. Average DNA methylation abundance across the entire genome did not significantly differ between groups (COVID-19 patients: 58.5%; non-COVID-19 patients: 58.4%, FIG. 4A), indicating no global changes in methylation abundance related to COVID-19 status. To investigate locus-specific DNA methylation levels linked to SARS-CoV-2 infection, methylome data were subjected to a linear regression model that accounted for age, sex, and leukocyte proportions for downstream analyses⁴⁴. This approach detected 254 DMRs distributed across the entire genome comprising clusters of ≥5 CpGs (FDR P-value <0.05; FIG. 4B; Table 6). A total of 101 hyper-methylated and 153 hypo-methylated DMRs were identified, indicating that COVID-19 patients demonstrate changes in specific DNA positions even when compared to patients with acute respiratory decompensation due to other causes. Mapping the 254 DMRs identified 230 annotated genes, including known immune function genes (e.g., IRF7, BCL6, MX1, and TNF). A gene ontological analysis identified significant enrichment of immune-related terms, including defense response to viruses, type I interferon signaling pathway constituents, and regulation of viral genome replication (FDR P-value <0.05; FIG. 4C; Table 7). Disease ontological terms disclosed significant links to other virus-causing diseases, including influenza and hepatitis C (FDR P-value <0.05; FIG. 4D; Table 8). These findings indicate that COVID-19 patients demonstrate an altered blood methylome compared to that of patients with respiratory illness arising from other causes, and that differences in DNA methylation occur at genes specific to COVID-19.

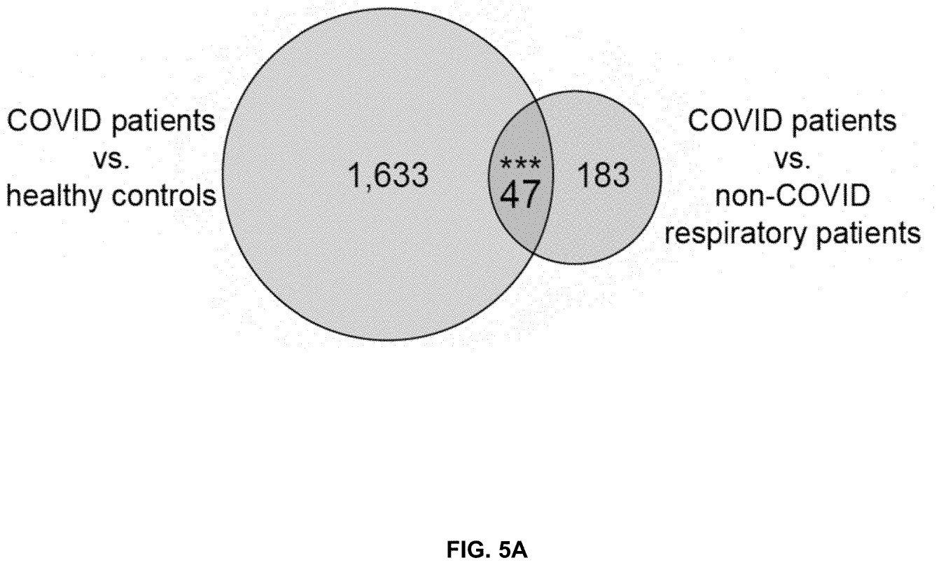

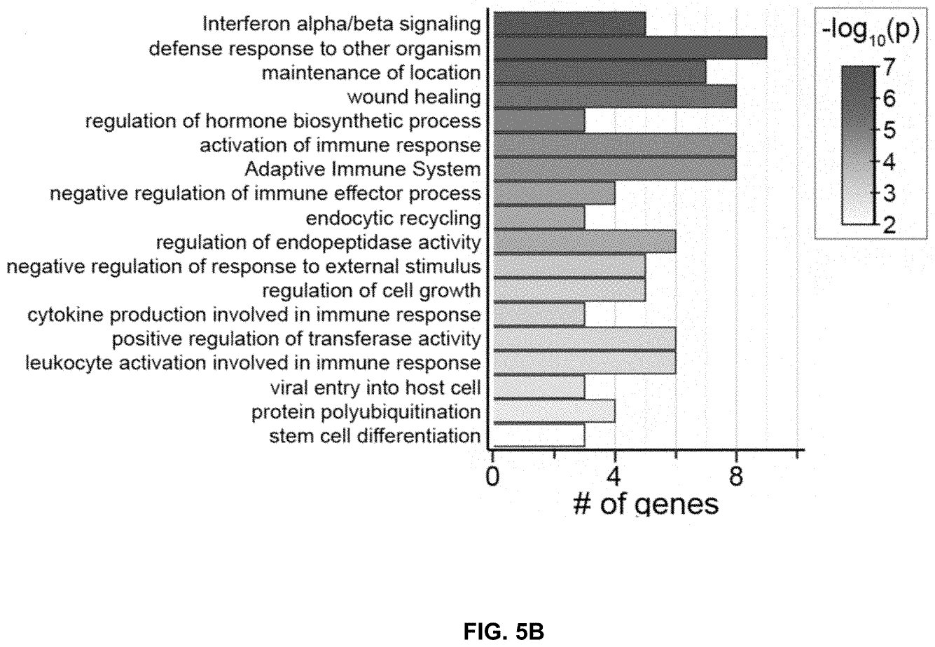

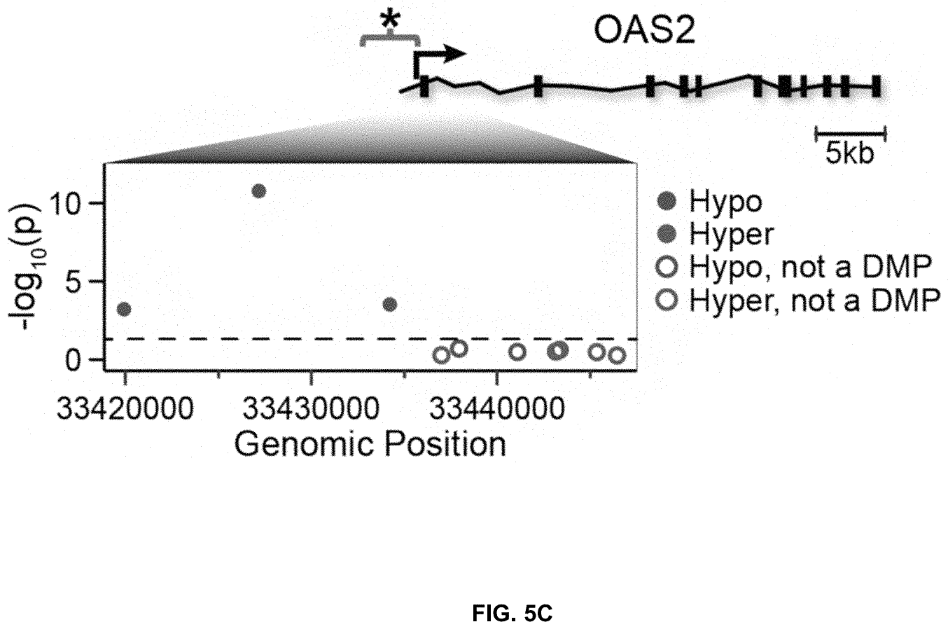

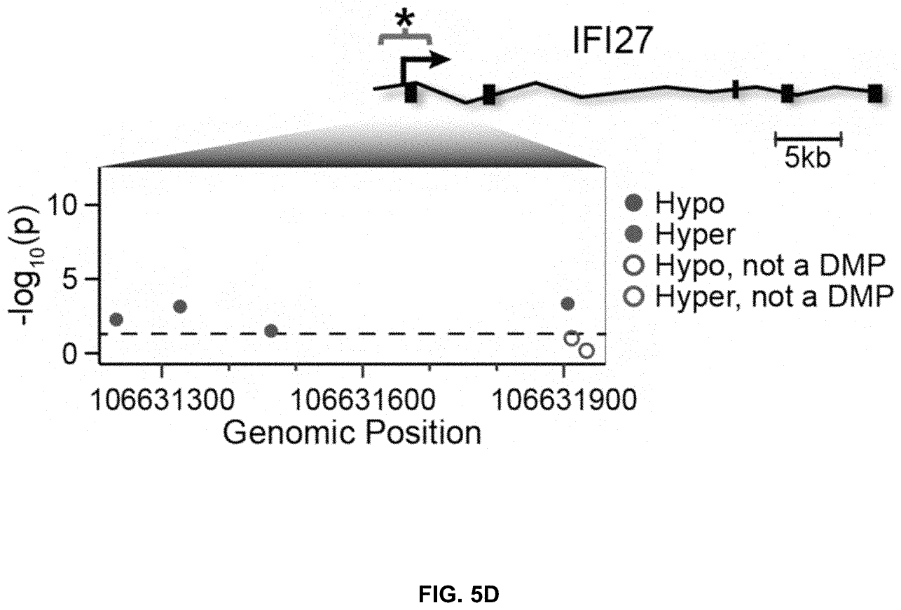

COVID-19 DNA methylation in blood and interferon-stimulated gene (ISG) expression. To narrow our focus on COVID-19 specific DMRs, we identified DMRs from COVID-19 patients vs. healthy pre-pandemic control individuals, and DMRs from COVID-19 patients vs. patients with non-COVID-19 respiratory illness. Forty-seven DMRs are shared between the 2 datasets (FIG. 5A; Table 9). Twenty-five of the 47 DMRs are closely linked to B lymphocyte, T lymphocyte, macrophage, and neutrophil functions, including antiviral activity, cytokine production, inflammation, and innate and adaptive immunity. Gene ontology and pathway enrichment analysis revealed significant enrichment in terms related to host defense responses including interferon alpha and beta signaling, defense response to organisms, and activation of the immune system (FIG. 5B). DMRs were hypo-methylated in promoter regions and contiguous sites in 2 prototypical interferon-stimulated genes, IFI27 and OAS2, (FIG. 5C-4D), suggesting possible regulatory effects on gene expression.

TABLE 9
Shared genes with COVID-specific DMRs between a) COVID-19
patients vs. healthy pre-pandemic controls, and b) between
COVID vs. non-COVID respiratory patients.
Overlapping Covid DMR-associated genes

AC010731.2
ACOT7
AIM2
AMICA1
ATP1A1
BCL6
C8ORF31
CDK2AP1
CLASP2
CPNE6
DCTN1-AS1
DDR1
DENND1B
EHD1
FBXO47
FGFBP2
FYB
GFI1
HTR2A
IFI27
IFIT3
IFITM1
LAT
LTBP1
MGRN1
NOTCH4
OAS2
PGLYRP1
PILRB
PLXNB2
PSMB8
RNF19A
RP11-290F20.3
RP11-323N12.5
RP11-546K22.1
S100A8
SEMA4A
SLC1A5
SLC27A3
SNORA38
SORL1
SP110
SP140
TNF
TNFRSF1A
VOPP1
VPS13D

To gain insight into the effects of DMRs on gene expression, we compared DMRs between COVID-19 patients and patients with non-COVID-19 respiratory illness, with differentially expressed genes (DEGs) identified in our RNAseq analysis of circulating leukocytes from the same patients¹¹. We identified 36 genes that were both differentially methylated and differentially expressed in COVID-19 patients (Table 10). This gene set was highly enriched in the gene ontology term: defense response to virus (27/36 genes) and a Reactome gene set: interferon signaling (19/36 genes) (Table 11; Table 12A and 12B). Eight in the interferon pathway were upregulated in parallel with the presence of DMRs in their genes. All identified DMRs were hypo-methylated with at least 5 consecutive CpGs near promoter regions (Table 11).

TABLE 10
Thirty-six genes that were both differentially methylated
and differentially expressed in COVID-19 patients.

| Methylation Status | Gene symbols | Methylation Location on Gene |
|---|---|---|
| Hypo | ADAR | Promoter (<=1 kb) |
| Hypo | CCDC110 | Promoter (<=1 kb) |
| Hypo | CCNT1 | Promoter (<=1 kb) |
| Hypo | CD38 | Promoter (<=1 kb) |
| Hypo | CLASP2 | Promoter (<=1 kb) |
| Hyper | COMT | Promoter (<=1 kb) |
| Hypo | DDX60 | Promoter (<=1 kb) |
| Hypo | DENND1B | Promoter (<=1 kb) |
| Hypo | DTX3L | Promoter (<=1 kb) |
| Hyper | EHD1 | Intron 1 of 4 |
| Hypo | EPSTI1 | Promoter (<=1 kb) |
| Hyper | GFI1 | Promoter (1-2 kb) |
| Hypo | HECTD4 | Promoter (<=1 kb) |
| Hypo | HERC5 | Promoter (<=1 kb) |
| Hyper | HTRA3 | Promoter (<=1 kb) |
| Hypo | IFI27 | Promoter (<=1 kb) |
| Hypo | IGSF11 | Promoter (<=1 kb) |
| Hyper | LAMB1 | Exon 24 of 32 |
| Hypo | LIN9 | Promoter (<=1 kb) |
| Hypo | LTBP1 | Promoter (<=1 kb) |
| Hypo | MX1 | Promoter (<=1 kb) |
| Hyper | NOTCH4 | Promoter (<=1 kb) |
| Hypo | OAS2 | Promoter (<=1 kb) |
| Hypo | PARP11 | Promoter (1-2 kb) |
| Hypo | PARP9 | Promoter (<=1 kb) |
| Hypo | PCSK4 | Promoter (<=1 kb) |
| Hypo | PPP4R4 | Promoter (<=1 kb) |
| Hypo | REEP6 | Promoter (<=1 kb) |
| Hypo | RNF19A | Promoter (<=1 kb) |
| Hyer | RNF39 | Promoter (1-2 kb) |
| Hypo | RSAD2 | Promoter (<=1 kb) |
| Hyper | SCNN1D | Promoter (<=1 kb) |
| Hypo | SPCS2 | Promoter (<=1 kb) |
| Hypo | SPRYD3 | Promoter (<=1 kb) |
| Hyper | VPS13D | Intron 67 of 69 |
| Hyper | ZBTB48 | Promoter (<=1 kb) |

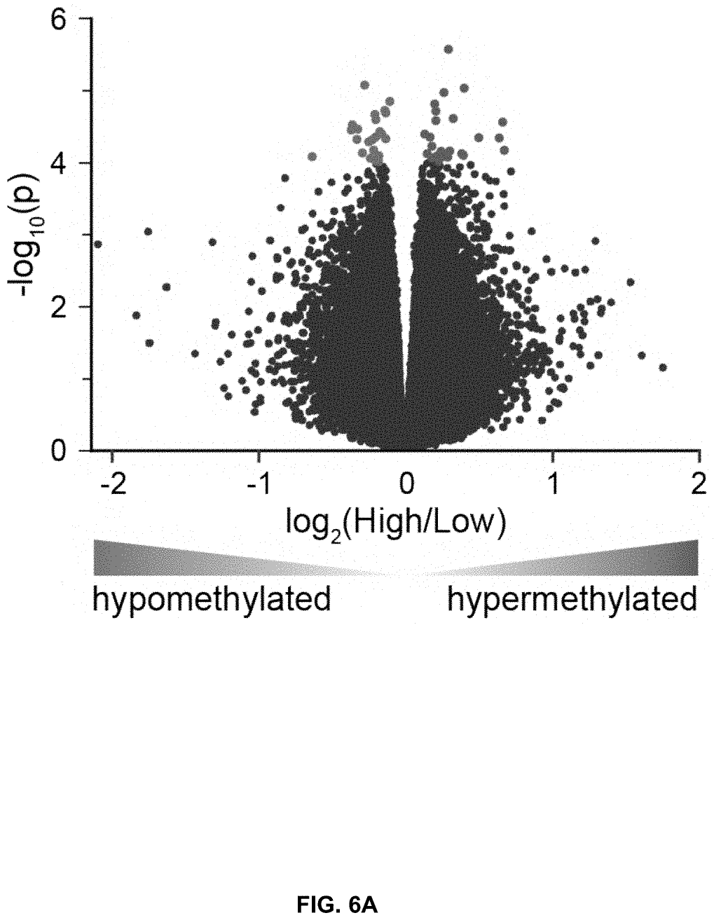

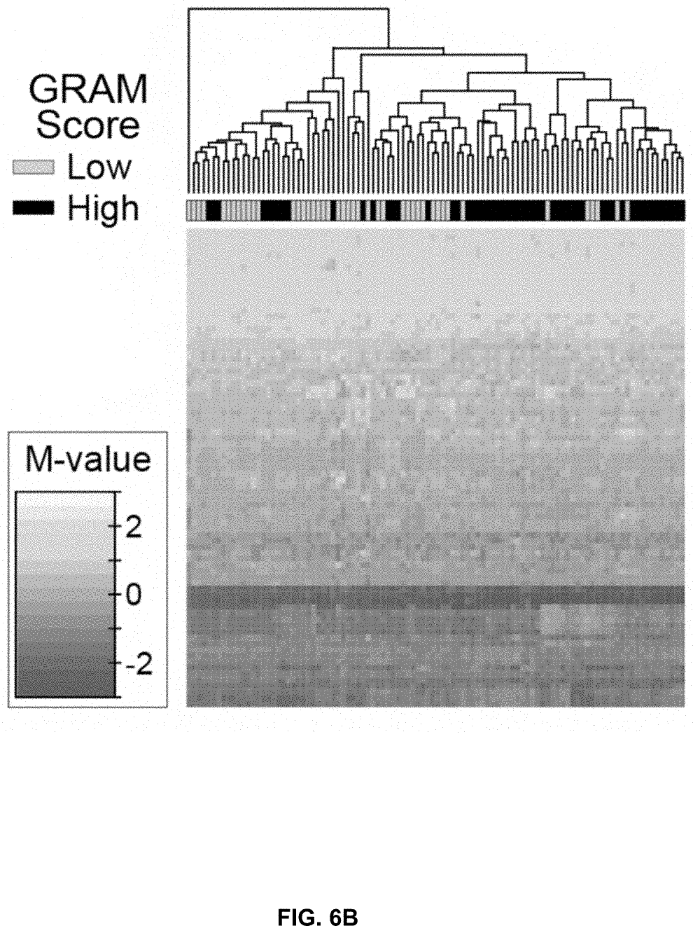

DNA methylation in blood and COVID-19 severity: The GRAM score is a validated outcome measure that defines the risk of deterioration in COVID-19 patients⁴⁰. We obtained GRAM scores and mortality outcomes in our cohort, which allowed comparison of different disease burdens with DMRs in blood, and to test the potential value of DMR analysis as a predictor of patient prognosis. The GRAM-score risk percentage was dichotomized into a discrete variable (i.e., low [<50%] and high [>50%]) and DNA methylation data was regressed on this variable in the COVID-19 respiratory patients (N=100). Because the GRAM-risk score has been validated for specific use in COVID-19 patients⁴⁰, only patients with COVID-19 were included in the analysis (Table 1). Nineteen DMRs with ≥3 consecutive differentially methylated CpGs were identified, (P-value <0.0001, Table 13) between patients with low and high GRAM-risk scores. In total, the DMRs comprised 145 differentially methylated positions (DMPs), of which there were 84% located at gene promoter regions and ˜65% were hyper-methylated (FIG. 6A). Evaluation of mortality as an outcome measure identified 18 DMRs comprising 113 DMPs, 62% of which were hyper-methylated (Table 14).

TABLE 13
DMRs from the COVID-19 GRAM-score risk prediction analysis.

| | | | | | Max | | |
|---|---|---|---|---|---|---|---|
| Chromo- | DMR | | #of | FDR | differential | Methylation | Gene |
| some | start | DMR end | CpGs | p-value | methylation* | direction | symbols |
| chr19 | 11784246 | 11785188 | 13 | 8.64E−12 | 0.06343425 | Hyper | |
| chr5 | 42756397 | 42757171 | 9 | 4.56E−09 | −0.129054 | Hypo | CCDC152 |
| chr11 | 2334892 | 2335053 | 4 | 9.53E−08 | 0.04305457 | Hyper | |
| chr2 | 47382287 | 47382903 | 11 | 2.56E−07 | 0.02795086 | Hyper | C2orf61 |
| chr1 | 162467080 | 162467363 | 5 | 3.61E−06 | 0.09203851 | Hyper | UHMK1 |
| chr19 | 52390810 | 52391789 | 14 | 5.08E−06 | 0.06479766 | Hyper | CTC- |
| | | | | | | | 429C10.2 |
| | | | | | | | ZNF577 |
| chr15 | 101389272 | 101389394 | 3 | 5.13E−06 | 0.08044284 | Hyper | RP11- |
| | | | | | | | 66B24.2 |
| | | | | | | | RP11- |
| | | | | | | | 66B24.7 |
| chr20 | 61583686 | 61584248 | 9 | 5.61E−06 | −0.0243455 | Hypo | SLC17A9 |
| chr7 | 965379 | 965534 | 3 | 5.65E−06 | 0.06265938 | Hyper | ADAP1 |
| chr5 | 138210550 | 138211184 | 13 | 6.91E−06 | 0.02071477 | Hyper | CTNNA1 |
| | | | | | | | LRRTM2 |
| chr2 | 27301195 | 27301943 | 9 | 8.24E−06 | −0.0516713 | Hypo | EMILIN1 |
| chr16 | 3355951 | 3356149 | 5 | 1.06E−05 | 0.06271509 | Hyper | HGD7 |
| | | | | | | | ZNF75A |
| chr1 | 40024971 | 40025415 | 4 | 1.60E−05 | 0.06345103 | Hyper | PPIEL |
| chr13 | 110438578 | 110439234 | 8 | 2.66E−05 | −0.0134248 | Hypo | IRS2 |
| chr1 | 95698827 | 95699097 | 6 | 5.72E−05 | −0.0677714 | Hypo | RP11- |
| | | | | | | | 57H12.3 |
| | | | | | | | RWDD3 |
| chr | 28829283 | 28829674 | 14 | 5.78E−05 | −0.0248236 | Hypo | RPL13P |
| | | | | | | | XXbac- |
| | | | | | | | BPG308K3.6 |
| chr5 | 102898463 | 102898733 | 8 | 6.32E−05 | −0.0585313 | Hypo | NUDT12 |
| chr6 | 168393930 | 168394160 | 3 | 8.55E−05 | 0.05638026 | Hyper | |
| chr11 | 2891077 | 2891118 | 4 | 8.60E−05 | 0.03952959 | Hyper | KCNQ1DN |

*Maximum differential methylation level for one of the CpGs in the DMR.

TABLE 14
DMRs from the survival of COVID-19 patient analysis.

| | DMR | | | FDR | Methylation | |
|---|---|---|---|---|---|---|
| Chromosome | start | DMR end | #of CpGs | p-value | direction | Gene symbols |
| chr13 | 112861499 | 112861518 | 3 | 1.07E−05 | Hypo | |
| chr10 | 76803669 | 76803925 | 3 | 4.22E−05 | Hyper | |
| chr1 | 63249197 | 63249213 | 4 | 1.26E−05 | Hyper | ATG4C |
| chr17 | 6797466 | 6797771 | 4 | 3.33E−05 | Hyper | ALOX12P2 |
| chr16 | 49563759 | 49564462 | 4 | 3.41E−09 | Hyper | |
| chr5 | 180257691 | 180257804 | 4 | 7.23E−05 | Hyper | LINC00847 |
| chr14 | 72053146 | 72053361 | 4 | 8.24E−05 | Hypo | SIPA1L1 |
| chr16 | 75568999 | 75569749 | 6 | 2.35E−08 | Hyper | CHST5 |
| chr2 | 121223534 | 121223964 | 6 | 4.40E−05 | Hyper | LINC01101 |
| chr7 | 151433178 | 151433561 | 6 | 4.07E−05 | Hyper | PRKAG2 |
| chr14 | 104394430 | 104394831 | 6 | 6.37E−06 | Hyper | TDRD9; C14or2 |
| chr2 | 27301195 | 27301943 | 9 | 7.73E−06 | Hypo | EMILIN1 |
| chr16 | 67034309 | 67034882 | 7 | 4.75E−08 | Hypo | CES4A |
| chr6 | 28058187 | 28059208 | 10 | 9.55E−11 | Hyper | ZSCAN12P1 |
| chr8 | 144635260 | 144635610 | 8 | 6.34E−06 | Hypo | GSDMD |
| chr5 | 110062384 | 110062618 | 8 | 8.24E−05 | Hypo | TMEM232 |
| chr6 | 32164503 | 32165200 | 10 | 5.42E−07 | Hyper | GPSM3; NOTCH4 |
| chr8 | 16859295 | 16860121 | 11 | 6.91E−11 | Hypo | FGF20 |

To identify specific DMPs that best define GRAM-score risk, the DNA methylation levels at these 145 GRAM-risk score-associated DMPs were subjected to a recursive feature elimination analysis⁴⁷. This algorithm revealed 77 DMPs with methylation levels that distinguish COV D-19 severity in a hierarchical cluster analysis (FIG. 63; Table 15). These data suggest that worse outcomes are associated with hyper-methylation in promoter regions and that specific positions throughout the genome may potentially correlate with COVID-19 severity.

TABLE 15
Seventy-seven DMPs with methylation levels
that distinguish COVID-19 severity.

| Illumina cg ID | Chromosome | Genomic position |
|---|---|---|
| cg19399165 | chr2 | 27301252 |
| cg00727912 | chr20 | 61583736 |
| cg00199007 | chr20 | 61583910 |
| cg11615743 | chr2 | 47382538 |
| cg01817521 | chr20 | 61584108 |
| cg04478428 | chr20 | 61584072 |
| cg10243676 | chr1 | 40024971 |
| cg21929564 | chr5 | 42757171 |
| cg03123541 | chr1 | 95699097 |
| cg14157549 | chr6 | 168393963 |
| cg19626861 | chr16 | 3356149 |
| cg26329715 | chr20 | 61583987 |
| cg12227172 | chr19 | 52391411 |
| cg14021880 | chr2 | 27301369 |
| cg22657016 | chr1 | 162467115 |
| cg00777011 | chr19 | 11784344 |
| cg08771171 | chr5 | 138211084 |
| cg20515447 | chr5 | 138210749 |
| cg16301975 | chr6 | 168394160 |
| cg22862450 | chr1 | 40025411 |
| cg20588660 | chr20 | 61584248 |
| cg05029532 | chr2 | 27301597 |
| cg16777413 | chr5 | 138211013 |
| cg05291965 | chr2 | 47382583 |
| cg15895197 | chr2 | 27301490 |
| cg14341579 | chr13 | 110438906 |
| cg13060480 | chr5 | 42757023 |
| cg17315014 | chr5 | 138211155 |
| cg17263716 | chr2 | 47382462 |
| cg14686919 | chr20 | 61584159 |
| cg11704876 | chr1 | 40025232 |
| cg16548362 | chr15 | 101389394 |
| cg22883889 | chr20 | 61583686 |
| cg17221377 | chr15 | 101389272 |
| cg11677683 | chr5 | 138211043 |
| cg17865528 | chr5 | 138210634 |
| cg07655627 | chr5 | 102898729 |
| cg05382022 | chr1 | 95698917 |
| cg02978008 | chr2 | 47382666 |
| cg09166085 | chr5 | 102898648 |
| cg07173972 | chr20 | 61583979 |
| cg12585923 | chr5 | 42756876 |
| cg18522909 | chr2 | 47382903 |
| cg19772114 | chr6 | 28829321 |
| cg19815863 | chr2 | 27301373 |
| cg26537280 | chr1 | 95699037 |
| cg14902598 | chr5 | 138210650 |
| cg17774102 | chr6 | 28829640 |
| cg15057250 | chr1 | 40025415 |
| cg18891081 | chr2 | 47382427 |
| cg14533206 | chr13 | 110439006 |
| cg03514843 | chr2 | 27301195 |
| cg20701556 | chr1 | 95698924 |
| cg02320481 | chr13 | 110438578 |
| cg08625260 | chr13 | 110439234 |
| cg18472410 | chr5 | 42756786 |
| cg05654832 | chr2 | 27301651 |
| cg01206378 | chr1 | 95698827 |
| cg22472290 | chr19 | 52391480 |
| cg15206834 | chr5 | 102898733 |
| cg05529874 | chr16 | 3356081 |
| cg20224754 | chr2 | 47382567 |
| cg06606949 | chr5 | 138211149 |
| cg24581408 | chr5 | 42756397 |
| cg20279471 | chr5 | 102898585 |
| cg02976617 | chr5 | 102898463 |
| cg19814518 | chr1 | 162467080 |
| cg04006194 | chr11 | 2335053 |
| cg25361850 | chr19 | 52391789 |
| cg23688479 | chr5 | 42756851 |
| cg21277452 | chr6 | 28829340 |
| cg20697204 | chr2 | 47382593 |
| cg00191629 | chr13 | 110439004 |
| cg22047282 | chr2 | 27301649 |
| cg01156295 | chr5 | 138211184 |
| cg25878441 | chr15 | 101389354 |
| cg00859178 | chr1 | 162467208 |

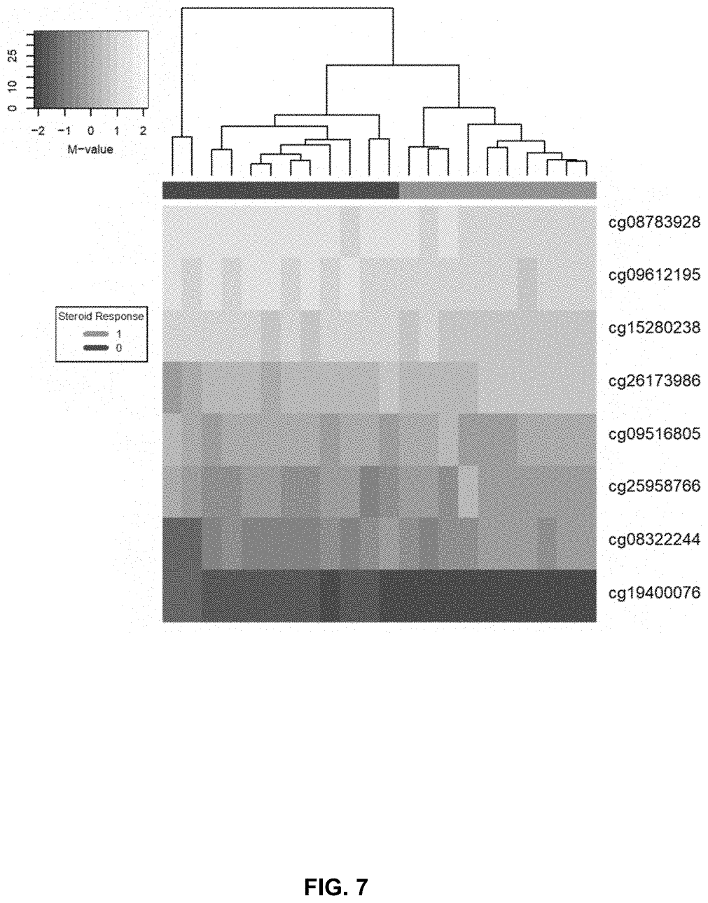

Corticosteroid response in COVID-19 patients: For the corticosteroid response comparison, we took all CpGs (1,281) residing in the DMRs (194, Table 16) found to be associated with “response to steroids” and subjected them to a recursive feature elimination algorithm to identify CpG DNA methylation levels that best distinguish steroid responders from non-responders. This approach identified 8 CpGs (cg08783928, cg25958766, cg26173986, cg09612195, cg19400076, cg09516805, cg15280238, and cg08322244) in a hierarchical cluster analysis (FIG. 7; Table 17A; Table 178).

TABLE 17A
Eight CpGs with methylation levels that best distinguish
steroid responders from non-responders.

| Illumina cg ID | Chromosome | Genomic position |
|---|---|---|
| cg19400076 | chr16 | 75681741 |
| cg08322244 | chr17 | 8066669 |
| cg25958766 | chr20 | 5485268 |
| cg09516805 | chr13 | 49107564 |
| cg26173986 | chr17 | 8067016 |
| cg15280238 | chr1 | 1.54E+08 |
| cg09612195 | chr12 | 15039253 |
| cg08783928 | chr17 | 8067323 |

TABLE 17B
DMRs encompassing the CpGs from Table 17B.

| Chromo- | DMR | DMR | # of | | Methylation |
|---|---|---|---|---|---|
| some | start | end | CpGs | P-value | direction |
| chr12 | 15037755 | 15039432 | 6 | 2.25E−42 | Hyper |
| chr20 | 5485144 | 5486007 | 10 | 3.64E−15 | Hyper |
| chr13 | 49107116 | 49108131 | 8 | 6.17E−11 | Hyper |
| chr17 | 8066669 | 8067323 | 4 | 1.99E−07 | Hyper |
| chr1 | 154127462 | 154128443 | 9 | 8.20E−07 | Hyper |
| chr16 | 75681737 | 75682004 | 5 | 3.47E−05 | Hyper |

### Discussion

In this prospective cohort study, we tested the hypothesis that COVID-19 patients demonstrate patterns of DNA methylation in blood that are different from pre-pandemic healthy individuals, and from patients with respiratory illness who did not have COVID-19. We also tested whether worse outcomes and steroid responsivity in COVID-19 patients are associated with DMRs and DMPs in blood.

DNA methylation in blood is altered in COVID-19 patients: In samples obtained within days of acute SARS-CoV-2 infection, patients exhibit 1089 (72%) hypo-methylated regions and 416 (28%) hyper-methylated regions comprising 5 or more consecutive differentially methylated CpGs in comparison with healthy control blood samples collected before the COVID-19 pandemic (FIG. 2B). While genome-wide DNA methylation data in circulating blood from a large cohort of patients with acute viral infection are not provided³², a recent report comparing patients with and without sepsis of unspecified origin indicates differential methylation at genes that participate in interferon-gamma-mediated (IFNg) signaling, MHCII antigen processing and presentation, immunoglobulin production, and cell adhesion pathways⁴⁸. In a limited study of 6 patients with SARS-CoV-2 infection, 6 DMPs (not DMRs) were observed in genes that encode proteins that participate in granulopoiesis and B-lymphocyte-to-granulocyte trans-differentiation¹⁰. Viral infections induce aberrant methylation patterns in host cells^{33,49}. For instance, H5N1 influenza and Middle Eastern respiratory syndrome coronavirus (MERS-CoV) infections down-regulate interferon-stimulated and antigen-presenting genes, which are associated with hyper-methylation of gene promoter regions in human airway epithelial cells in vitro^{28,29}. The large number of DMRs identified by the very conservative criteria and inferential comparisons that we've discovered, and the diversity of their corresponding loci and pathways, are surprising in view of the short interval from infection to hospitalization in the enrolled patients, thereby denoting the role of the methylome as a rapid responder to SARS-CoV-2 infection. Interestingly, a very recent report focused on pediatric critically illness demonstrates a rapid regulation of DNA methylation in circulating leukocytes, taking place within the first three days of hospitalizations⁵⁰.

Genes comprising DMRs between patients with COVID-19 and healthy pre-pandemic controls include IFN-stimulated genes (ISGs), with well-recognized antiviral activity such as IFI27 and OAS2. Differential methylation of type I IFN pathway genes in specific leukocyte subsets is associated with autoimmune disorders including Sjogren's syndrome, Lupus, Grave's disease, and rheumatoid arthritis⁵¹⁻⁵⁵, indicating a role for ISG methylation in the dysregulation of inflammatory processes, and autoimmunity as a contributor to COVID-19 pathogenesis^{56,57}. Less is known about the impact of ISG methylation in blood on the control of viral infections. Recently, a correlation between ISG methylation and the outcome of HIV infection has been reported, with hyper-methylation of interferon and antiviral genes correlated with improved HIV control⁵⁸. In SARS-CoV-2 infection, differential methylation and expression of antiviral ISGs may influence viral replication and spread in leukocyte subsets⁵⁹, or contribute to COVID-19 pathogenesis by altering immune cell activation or function. Multiple DMRs reported here appear in genes recently found with dysregulated expression levels in samples from the same patients¹¹. Of note, these upregulated ISGs were differentially hypo-methylated in gene promoter regions, suggesting that methylation contributes to transcriptional regulation.

DNA methylation in blood and COVID-19 severity: To determine if disease severity in COVID-19 patients is associated with DMRs in blood, we tested the association of DMRs with clinical outcomes including the GRAM risk score⁴⁰ and mortality. We found that worse GRAM scores were associated with 19 DMRs comprising 145 differentially methylated positions (DMPs) in 18 genes. Sixty-three percent of the GRAM-score-associated DMPs were hyper-methylated. Mortality was associated with 18 DMRs comprising 113 DMPs in 17 genes (Table 14). In this setting, 61% of the DMRs were hyper-methylated. Over 84% of the DMRs associated with outcomes were located in gene promoter regions; notably, promoter, hyper-methylation is associated with transcriptional repression^{15,16,18}. Previous research indicates that non-permissive (immunosuppressive) transcriptomic states are associated with worse outcomes in critical illness^{30,60-62}. Moreover, protracted COVID-19 is associated with blockade of T-cells proliferation⁶³ and suppression of the innate immune system in circulating blood¹³.

Using recursive feature elimination, we identified 77 DMPs that discriminate clinical outcomes. Such an approach predicts patients at risk for clinical decompensation, and thereby improves resource allocation and support early intervention. Analyses using whole genome methylation sequencing can supplement these data.^{11,64,65}.

General considerations: While the global RNA transcriptomic profiles in blood have been previously reported in sepsis³⁰, acute respiratory distress syndrome^{31,66} and COVID-19^{10,11,13}, and there are recently reported small cohorts describing blood DNA methylation in COVID-19⁶⁷, no prior reports compare differentially methylated regions (i.e., DMRs not just DMPs) in blood samples from COVID-19 patients to samples collected before the SARS-CoV-2 pandemic using a shared epigenotyping platform together with predictors of severity, outcomes, and therapeutic responsivity. Together, DNA methylation and RNA expression data will facilitate improved COVID-19 diagnosis, prognosis and targeted treatments⁶⁸. COVID-19-induced DMRs may persist long after acute care, contributing to the post-ICU syndrome comprising physical and cognitive dysfunction⁶⁹⁻⁷². Recent data indicate that blood DNA methylation profiles mediate worse neurocognitive development in the pediatric ICU population²⁵, which is relevant in COVID-19 as well^{73,74}.

Methodological considerations: Use of the Illumina Infinium MethylationEPIC 850,000 BeadChip facilitates comparisons of data between investigations that employ a shared platform comprising sites that span the genome. This approach, which predominantly captures circulating leukocytes DNA, has been recently used in the intensive care setting²⁵. The use of mixed cell populations in whole blood is of high relevance in infectious disease diagnosis and prognosis⁶⁴, and has supported identification of actionable subphenotypes^{34,35,68}. Whereas the nucleotide sequence of the genome is remarkably stable from conception to death^{15,16}, our data demonstrate that DNA methylation is rapidly dynamic, influences the expression of genes that regulate COVID-19 progression¹¹, and potentially modifiable by acute insults which could be reversed by targeted interventions⁷⁷.

### Materials and Methods

### Cohort Characteristics.

Human subject enrollment: Albany Medical Center: We conducted a single-center observational study of adult subjects admitted to either the medical floor or the medical intensive care unit (MICU) of Albany Medical Center in Albany, N.Y. Enrollment took place between Apr. 6, 2020 and May 1, 2020 and follow-up continued until Jun. 15, 2020. Patients were eligible for enrollment if they were older than 18 years and were admitted to the hospital for symptoms compatible with COVID-19. Exclusion criteria were imminent death or inability to provide consent, which was obtained from the patient or a legally authorized representative. Patients were assigned to the COVID-19 group only after receiving a positive test result via nasopharyngeal swab testing using the Abbott Realtime SARS-CoV-2 Assay® (Abbott, Ill.). SARS-CoV-2 test negative participants were assigned to the non-COVID-19 respiratory patient group as controls. The cause of respiratory distress in the non-COVID-19 patients is presented in Table 2. Pre-hospital co-morbidities determined using clinical history and hospital documentation were aggregated using the Charlson comorbidity index⁴³. APACHE II, SOFA, and SAPS II scores were used to assess severity of critical illness on ICU admission⁴². Sex, age, and other relevant subject data are provided in Table 1 and 2.

Blood samples were collected before 2017 from 39 healthy normal control participants. Participants were recruited from the community by advertisements and outreach events, and served as healthy normal controls in a Wisconsin Alzheimer's Disease Research Center (WADRC) investigation²¹. The healthy normal control participants complete a yearly study visit consisting of a blood draw, medical history questionnaires, psychometric testing, a physical exam, and must have no known diseases that interfere with study participation over time. Demographic details of the healthy normal control participants are provided in Table 1.

Selection of outcome measures. We analyzed the data with an outcome measure that: 1) is able to combine the severity of disease with mortality in a single metric; 2) is applicable to both ICU and medical floor populations; 3) uses a timeframe that accounts for longer hospitalizations in COVID-19 patients with respiratory failure compared with non-COVID-19 individuals^{3,78}; 4) accounts for COVID-19 linear deterioration that transitions from mild respiratory compromise to respiratory failure, followed by respiratory distress requiring mechanical ventilatory support and eventually death. Thus, we selected the composite outcome variable defined by the COVID-19 risk GRAM score⁴⁰. Characteristics contributing to the determination of the COVID-19 risk GRAM score are shown in Table 18-19. To simplify the analysis, patients were separated into two groups based on a calculated risk percentage below or above 50%. The secondary outcome measure was in-hospital mortality.

TABLE 18
Demographics and baseline characteristics of discrete Covid-GRAM risk percentage

| | Total | | Risk of severe outcome | | | | |
|---|---|---|---|---|---|---|---|
| | n = 102 | | Under 50% | | Over 50% | | |
| Variables | n = 102 | | n = 57 | | n = 45 | | P-value |
| Outcome | | | | | | | |
| Survived | 77 | (75.5%) | 54 | (94.7%) | 23 | (51.1%) | <0.001* |
| Died | 25 | (24.5%) | 3 | (5.3%) | 22 | (48.9%) | <0.001* |
| Sex - n(%) | | | | | | | |
| Male | 64 | (62.7%) | 35 | (61.4%) | 29 | (64.4%) | 0.75 |
| Female | 38 | (37.3%) | 22 | (38.6%) | 16 | (35.6%) | 0.75 |
| Age-year | | | | | | | |
| Mean (IQR) | 61.3 | (50.3-74.0) | 55.0 | (46.0-66.0) | 69.2 | (62.0-79.0) | <0.001* |
| Ethnicity - n (%) | | | | | | | |
| White | 46 | (45.1%) | 22 | (38.5%) | 24 | (53.3%) | 0.14 |
| Black | 11 | (10.8%) | 5 | (8.8%) | 6 | (13.3%) | 0.46 |
| Asian | 2 | (1.9%) | 1 | (1.8%) | 1 | (2.2%) | 0.87 |
| Hispanic | 21 | (20.6%) | 16 | (28.1%) | 5 | (11.1%) | 0.04* |
| Other | 22 | (21.6%) | 13 | (22.8%) | 9 | (20.1%) | 0.73 |
| BMI, kg/m2 Mean | 30.4 | (25.5-32.2) | 30.4 | (26.7-32.4) | 30.3 | (24.0-31.6) | 0.92 |
| (IQR) | | | | | | | |
| Severity Indexes | | | | | | | |
| (IQR) | | | | | | | |
| Charlson | 3.3 | (1.0-5.0) | 2.4 | (1.0-4.0) | 4.5 | (3.0-7.0) | <0.001* |
| comorbidity index | | | | | | | |
| Covid-GRAM | 154.3 | (118.0-181.0) | 114.9 | (105.0-130.0) | 204.1 | (169.0-218.0) | <0.001* |
| Biomarkers (IQR) | | | | | | | |
| Ferritin (ng/mL) | 938.9 | (303.3-1201.3) | 750.9 | (206.0-1116.0) | 1170.6 | (436.0-1215.5) | 0.06 |
| C-Reactive protein | 140.9 | (52.5-203.7) | 113.3 | (43.4-149.6) | 173.8 | (93.6-254.3) | 0.003* |
| (mg/L) | | | | | | | |
| D-dimer (mg/L FEU) | 11.7 | (1.1-12.5) | 5.5 | (0.7-4.7) | 18.3 | (1.4-18.9) | 0.007* |
| Procalcitonin | 3.2 | (0.2-1.7) | 1.3 | (0.1-1.28) | 5.2 | (0.3-1.8) | 0.07 |
| (ng/mL) | | | | | | | |
| Lactate (mmol/L) | 1.2 | (0.9-1.5) | 1.1 | (0.8-14) | 1.4 | (1.0-1.6) | 0.06 |
| Fibrinogen (mg/dL) | 543.6 | (414.0-659.0) | 550.1 | (410.5-703.0) | 537.3 | (438.0-654.0) | 0.77 |
| Albumin (mg/L) | 2.9 | (2.6-3.3) | 3.0 | (2.7-3.4) | 2.8 | (2.5-3.1) | 0.02* |
| Hemogram (IQR) | | | | | | | |
| White blood cells | 10.8 | (6.1-12.4) | 8.5 | (5.1-10.2) | 13.6 | (7.1-15.0) | 0.003* |
| (K/μL) | | | | | | | |
| Hemoglobin (g/dL) | 11.2 | (9.7-12.6) | 11.5 | (10.1-12.7) | 10.7 | (9.4-12.5) | 0.04* |
| Mean corpuscular | 87.1 | (84.6-93.6) | 86.8 | (84.5-93.0) | 87.5 | (86.1-94.1) | 0.78 |
| volume (fL) | | | | | | | |
| Platelet (K/μL) | 266.0 | (195.0-318.0) | 280.0 | (225.0-338.0) | 248.2 | (166.0-309.0) | 0.18 |
| Neutrophils (%) | 76.2 | (69.0-86.0) | 71.1 | (64.0-83.0) | 82.7 | (79.0-90.0) | <0.001* |
| Lymphocytes (%) | 13.8 | (5.0-18.0) | 18.1 | (8.0-24.0) | 8.4 | (4.0-10.0) | <0.001* |
| Monocytes (%) | 7.1 | (4.0-9.0) | 8.0 | (6.0-9.0) | 5.9 | (3.0-8.0) | 0.02* |
| Eosinophils (%) | 0.8 | (0.0-1.0) | 1.1 | (0.04.0) | 0.4 | (0.0-1.0) | 0.02* |
| Treatment - n (%) | | | | | | | |
| Hydroxychloroquine | 87 | (85.3%) | 50 | (87.7%) | 37 | (82.2%) | 0.44 |
| Antibiotics | 98 | (96.1%) | 54 | (94.7%) | 44 | (97.8%) | 0.43 |
| Antiviral | 1 | (0.9%) | 1 | (1.8%) | 0 | (0.0%) | 0.37 |
| IL6- Antagonist | 4 | (3.9%) | 2 | (3.5%) | 2 | (4.4%) | 0.81 |
| Convalescent Plasma | 26 | (25.5%) | 10 | (17.5%) | 16 | (35.6%) | 0.04* |
| Steroids | 46 | (45.1%) | 20 | (35.1%) | 26 | (57.8%) | 0.02* |

Sample collection and storage: At enrollment, blood samples were collected using BD EDTA Vacutainers®. Whole blood was then aliquoted and frozen at −80 C degrees for later processing and analysis.

DNA isolation and methylation microarray: DNA was isolated from 500 μL of frozen whole blood using the GeneJET whole blood kit (Thermo Fisher Scientific, K0782) following manufacturer's protocols. DNA concentration was determined using a Qubit fluorometer (Thermo Fisher Scientific) and normalized to 20 ng/μL for microarray analysis. Samples were shipped overnighted to Genuity Science Inc. (Boston, Mass.) for bisulfite conversion and methylation microarray analysis using the Illumina Infinium MethylationEPIC Beadchip array⁷⁹. The shared collection and processing of the blood DNA methylation levels from the Wisconsin healthy individuals' cohort (WADRC) was previously published²¹.

Illumina Human MethylationEPIC data preprocessing: To identify methylation changes associated with COVID-19, we compared COVID-19 patients (N=102) to methylation data from pre-pandemic participants²¹ that were enrolled 3 or years before the SARS-CoV-2 outbreak (N=39). Raw .idat files from all (N=141) were imported to the R environment. R package minfi was used to parse and preprocess methylation microarray data⁸⁰. The quality of raw data was assessed, and no samples were filtered due to high mean detection P-value (i.e., mean >0.05). Bisulfite conversion of samples was assessed for each sample by density and bean plots, and determinations, to assure that the distribution of beta-values were bimodal with the largest densities being centered on 0 or 1, and that the majority of data was either methylated or unmethylated. All samples were deemed to be successfully converted. Leukocyte proportions were estimated from methylation signatures, and cell counts were extracted for incorporation into models of differential methylation. Samples were normalized using functional normalization by background and dye correction following the normal-exponential out-of-band method⁸¹. Following normalization, sex prediction was generated using normalized values.

Two COVID-19 samples were removed due to improper sex prediction from the COVID-19 and non-COVID-19 cohorts each, suggesting unreliable methylation values from these samples. Probes were removed from remaining samples (N=139) if any of the following criteria applied: probes measured methylation on sex chromosomes; probes contained or reported methylation at SNPs; probes measured methylation at CH sites; detection P-value of a probe >0.01 for at most one sample; and probes were known to be cross-reactive. This filtering approach removed 99,905 probes through quality processing, leaving 765,954 for further analysis. Beta-values and log it M-values from the remaining probe set were generated for differential analysis. A one-way ANOVA was used to determine significant differences between mean beta-values of patients between groups.

To identify methylation changes associated specifically with COVID-19 versus non-COVID-19 respiratory patients, or other variables of interest (i.e., GRAM score, and mortality), raw .idat files from the AMC cohort (N=124) samples were imported to the R environment. R package minfi was used to parse and preprocess methylation microarray data⁸⁰. The quality of raw data and bisulfite conversion were assessed, leukocyte proportions were estimated, and samples were normalized, as above. After normalization, sex prediction was generated using normalized values. Four samples were removed due to improper sex prediction, suggesting unreliable methylation values for these samples. Probes were removed from remaining samples (N=124) using the criteria as above. Filtering removed 95,447 probes through quality processing, leaving 770,412 for further analysis. Beta-values and log it M-values were generated for differential analysis. A one-way ANOVA was used to determine significant differences between mean beta-values of patients between groups.

Model selection for differential analysis: Several potential models using available covariates were assessed to generate the best fit for the data. To compare COVID-19 (N=100) samples with pre-pandemic samples (N=39), models accounting for COVID-19 status (positive vs. negative), age, sex, and estimated leukocyte proportions (i.e., granulocytes, monocytes, natural killer cells, B lymphocytes, CD8 T lymphocytes, CD4 T lymphocytes) were generated. Model selection was based on BIC score criterion. Of the tested models, a model accounting for COVID-19 status, sex, and leukocyte proportions was preferential and used for downstream analyses. Batch effects between microarrays were adjusted using ComBat from the R package sva⁸². Batch-adjusted beta- and M-values were assessed by the R package sva to identify unknown confounders such as with other infections or complications. The surrogate variables found were adjusted for during model fitting.

To compare COVID-19 respiratory patients (N=100) with non-COVID-19 respiratory patients (N=24), the model selection was performed as above.

When assessing methylation levels associated with mortality of COVID-19 patients (N=100) and GRAM score (N=100), model selection was performed as above. Based on BIC criterion, models adjusted for surrogate variables using sva were selected for downstream analysis. Two outlier samples were removed from the GRAM score analysis because their scores were greater than 3 standard deviations from the mean.

Detection of differentially methylated regions: R package DMRcate was used for the detection of differentially methylated regions (DMRs)⁴⁴. M-value matrices were annotated to their chromosomal position, and test statistics were generated for variables of interest using models as described above. For comparisons of COVID-19 patients vs. pre-pandemic healthy participants, and COVID-19 patients vs. non-COVID respiratory patients, DMRs were identified using an FDR P-value cutoff of 0.05 and a minimum of 5 CpG sites in the region. For the comparison of methylation levels to GRAM score, and mortality, criteria for DMR identification included a P-value cutoff of 0.0001 and a minimum of 3 CpG sites in the region. Genes annotated to DMRs were extracted for downstream ontological analyses.

Ontological analyses: Genes comprising DMRs were assessed for ontological analyses of biological processes and diseases using the R package clusterProfiler⁸³. A listing of background genes was generated from all tested regions from DMRcate (N=20,899 genes). Gene symbols were converted to ENTREZIDs. Significant terms were determined using an FDR P-value cutoff of 0.05, comparing differentially methylated genes to the background gene list.

Plot generation: Manhattan plot generation used R packages qqman and ggplot2. For the pie plot, R package ChIPseeker was used to annotate regions⁸³. Bar plots of ontological terms were generated using the R package clusterProfiler. Hypergeometric tests in the R environment were used to identify enrichments of gene lists. Customized Circos plots were generated using the R package BioCircos⁸⁴. For heatmap generation of dichotomous GRAM score data, the R package caret was used for backwards feature selection, starting with a matrix of M-values from all CpGs in identified DMRs from the comparison. For model selection, cross-validation methodology and 5 iterations using subsets of 1 to 100 CpGs were used. Heatmaps were generated using the R packages gplots and heatmap.plus.

Data availability: Raw .idat files data are available at GEO accession GSE174818.

### Primer Design.

The following protocol can be employed to interrogate the abundance of 5 mC and/or 5 hmC in regions of interest. Once regions of interest are identified, genomic sequences corresponding to each locus can be obtained from a reference genome and imported to a methyl primer design software package, such as the Thermo Fisher Scientific Methyl Primer Express v1.0 software, to generate high quality PCR primers pairs for methylation mapping. Ultimately, a second pair of primers can be designed that contains the initial primer sequence flanked by an Illumina adapter sequence. Primers can be ordered through any preferred provider.

### Methyl Primer Express™ Software v1.0 (Thermo Fisher Scientific) Design Primer

1. Download Methyl Primer Express from the Thermo Fisher Scientific Applied Biosystems website. (thermofisher.com/us/en/home.html)
2. Input target sequence into the box labeled Insert Nucleotide Sequence.
   To increase the likelihood of finding primers that will create an amplicon of ˜200 base pairs (bp) or larger, be sure to input a minimum sequence of ˜500 base pairs.
   The longer the input sequence, the better chance of finding suitable primers to target the region of interest.
3. Click on Design Primers. Choose Select Target Sequence to select the specific region of interest within the input DNA sequence.
   Note that as above, the longer the target sequence selected, the more likely it is to find suitable primers.
4. Following selection of primers, press Next and a new window will open.
5. If finding CpG islands within the target sequence is of interest, click Find CpG Islands.
   Be sure to check the current default parameters that define a CpG island. If the input sequence is only 500 base pairs, consider shortening the defined minimum length of the island, as the length of a CpG island will later serve to help in choosing where to design the primers.
   It is possible that these analyses may not yield any CpG islands. If this is the case, simply move on to the next step.
6. In the bottom right corner of the new window, select bisulfite sequencing primer design by clicking Design BSP Primers.
   BSP=Bisulfite Sequencing Primers.
   MSP=Methylation-Specific Primers.
7. Review parameters used to design the BSPs by clicking Next.
   Ideally, design primers that will generate an amplicon length of 200 to 300 base pairs.
   Amplicons <200 base pairs will result in sequencing the adaptors, meaning it will not maximize the data from the final sequencing run.
   Primers should be between 20 to 30 base pairs in length. Annealing temperature of the PCR should have a small range (e.g., 58° to 60° C.).

### Support Protocol

8. Initially weight performance towards the Low Speed/High Accuracy setting (between 8 to 10). If no appropriate primers were found, move the scale towards the Hi Speed/Low Accuracy; however, this is not ideal.
9. Log on to a standard primer provider's webpage.
10. Go to the Order Menu and click Custom Synthesis. Press Order in the row beginning with “25 nmole DNA”.
11. Order Primers with the following parameters through any preferred provider:
   a. Select synthesis scale: 25 nmole DNA oligo.
   b. Select purification: Standard Desalting.
   c. Select dilutant: RNase Free Water (unless shipped desiccated).
   If applicable, design two to three primer pairs per target region, which can be tested simultaneously.

TABLE 20
SEQ ID NOs of DNA sequences used for primer design and exemplary primers.

| | SEQ ID NO | | | | |
|---|---|---|---|---|---|
| Genomic DNA | | Bisulfite | | | Primer-Targeted |
| Range for Primer | Original | Converted | Forward | Reverse | Genomic DNA |
| Design | DNA | DNA | Primer | Primer | Range |
| Chr1: 40024721- | 1 | 2 | 3 | 4 | Chr1: 40024971- |
| 40025665 | | | | | 40025415 |
| Chr1: 63248947- | 5 | 6 | 7 | 8 | Chr1: 63249197- |
| 63249463 | | | | | 63249213 |
| Chr1: 95698577- | 9 | 10 | 11 | 12 | Chr1: 95698827- |
| 95699347 | | | | | 95699097 |
| Chr1: 154127212- | 13 | 14 | 15 | 16 | Chr1: 154127462- |
| 154128693 | | | 17 | 18 | 154128443 |
| Chr1: 162466830- | 19 | 20 | 21 | 22 | Chr1: 162467080- |
| 162467613 | | | | | 162467363 |
| Chr2: 27300945- | 23 | 24 | 25 | 26 | Chr2: 27301195- |
| 27302193 | | | 27 | 28 | 27301943 |
| Chr2: 47382037- | 29 | 30 | 31 | 32 | Chr2: 47382287- |
| 47383153 | | | 33 | 34 | 47382903 |
| Chr2: 121223284- | 35 | 36 | 37 | 38 | Chr2: 121223534- |
| 121224214 | | | | | 121223964 |
| Chr5: 42756147- | 39 | 40 | 41 | 42 | Chr5: 42756397- |
| 42757421 | | | 43 | 44 | 42757171 |
| Chr5: 102898213- | 45 | 46 | 47 | 48 | Chr5: 102898463- |
| 102898983 | | | | | 102898733 |
| Chr5: 110062134- | 49 | 50 | 51 | 52 | Chr5: 110062384- |
| 110062868 | | | | | 110062618 |
| Chr5: 138210300- | 53 | 54 | 55 | 56 | Chr5: 138210550- |
| 138211434 | | | | | 138211184 |
| Chr5: 180257441- | 57 | 58 | 59 | 60 | Chr5: 180257691- |
| 180258054 | | | | | 180257804 |
| Chr6: 28057937- | 61 | 62 | 63 | 64 | Chr6: 28058187- |
| 28059458 | | | 65 | 66 | 28059208 |
| Chr6: 28829033- | 67 | 68 | 69 | 70 | Chr6: 28829283- |
| 28829924 | | | | | 28829674 |
| Chr6: 32164253- | 71 | 72 | 73 | 74 | Chr6: 32164503- |
| 32165450 | | | | | 32165200 |
| Chr6: 168393680- | 75 | 76 | 77 | 78 | Chr6: 168393930- |
| 168394410 | | | | | 168394160 |
| Chr7: 965129- | 79 | 80 | 81 | 82 | Chr7: 965379- |
| 965784 | | | | | 965534 |
| Chr7: 151432928- | 83 | 84 | 85 | 86 | Chr7: 151433178- |
| 151433811 | | | | | 151433561 |
| Chr8: 16859045- | 87 | 88 | 89 | 90 | Chr8: 16859295- |
| 16860371 | | | 91 | 92 | 16860121 |
| Chr8: 144635010- | 93 | 94 | 95 | 96 | Chr8: 144635260- |
| 144635860 | | | | | 144635610 |
| Chr10: 76803419- | 97 | 98 | 99 | 100 | Chr10: 76803669- |
| 76804175 | | | | | 76803925 |
| Chr11: 2334642- | 101 | 102 | 103 | 104 | Chr11: 2334892- |
| 2335303 | | | | | 2335053 |
| Chr11: 2890827- | 105 | 106 | 107 | 108 | Chr11: 2891077- |
| 2891368 | | | | | 2891118 |
| Chr12: 15037505- | 109 | 110 | 111 | 112 | Chr12: 15037755- |
| 15039682 | | | 113 | 114 | 15039432 |
| | | | 115 | 116 | |
| Chr13: 49106866- | 117 | 118 | 119 | 120 | Chr13: 49107116- |
| 49108381 | | | 121 | 122 | 49108131 |
| Chr13: 110438328- | 123 | 124 | 125 | 126 | Chr13: 110438578- |
| 110439484 | | | | | 110439234 |
| Chr13: 112861249- | 127 | 128 | 129 | 130 | Chr13: 112861499- |
| 112861768 | | | | | 112861518 |
| Chr14: 72052896- | 131 | 132 | 133 | 134 | Chr14: 72053146- |
| 72053611 | | | | | 72053361 |
| Chr14: 104394180- | 135 | 136 | 137 | 138 | Chr14: 104394430- |
| 104395081 | | | | | 104394831 |
| Chr15: 101389022- | 139 | 140 | 141 | 142 | Chr15: 101389272- |
| 101389644 | | | | | 101389394 |
| Chr16: 3355701- | 143 | 144 | 145 | 146 | Chr16: 3355951- |
| 3356399 | | | | | 3356149 |
| Chr16: 49563509- | 147 | 148 | 149 | 150 | Chr16: 49563759- |
| 49564712 | | | | | 49564462 |
| Chr16: 67034059- | 151 | 152 | 153 | 154 | Chr16: 67034309- |
| 67035132 | | | | | 67034882 |
| Chr16: 75681487- | 155 | 156 | 157 | 158 | Chr16: 75681737- |
| 75682254 | | | | | 75682004 |
| Chr16: 75568749- | 159 | 160 | 161 | 162 | Chr16: 75568999- |
| 75569999 | | | 163 | 164 | 75569749 |
| Chr17: 6797216- | 165 | 166 | 167 | 168 | Chr17: 6797466- |
| 6798021 | | | | | 6797771 |
| Chr17: 8066419- | 169 | 170 | 171 | 172 | Chr17: 8066669- |
| 8067573 | | | 173 | 174 | 8067323 |
| Chr19: 11783996- | 175 | 176 | 177 | 178 | Chr19: 11784246- |
| 11785438 | | | 179 | 180 | 11785188 |
| Chr19: 52390560- | 181 | 182 | 183 | 184 | Chr19: 52390810- |
| 52392039 | | | 185 | 186 | 52391789 |
| Chr20: 5484894- | 187 | 188 | 189 | 190 | Chr20: 5485144- |
| 5486257 | | | 191 | 192 | 5486007 |
| Chr20: 61583436- | 193 | 194 | 195 | 196 | Chr20: 61583686- |
| 61584498 | | | 197 | 198 | 61584248 |

### Example 2

### Summary

We recently reported the COVID-19-induced circulating leukocytes DNA methylation profile. Here, we hypothesized that some of these genes would remain differentially methylated after disease resolution. Fifteen participants previously hospitalized for SARS-CoV-2 infection were sequenced one year after discharge. Of the 1,505 acute illness induced differentially methylated regions (DMRs) previously identified, we found 71 regions that persisted differentially methylated, with an average of 7 serial CpG positions per DMR. Sixty-four DMRs remained hypermethylated, and 7 DMR remained hypomethylated. These data are the first reported evidence that DNA methylation changes in circulating leukocytes endure long after recovery from acute illness.

### Background

The COVID-19 pandemic has caused 6 million deaths worldwide. Many COVID-19 survivors fail to recover their pre-infection status, with lasting physical impairments and increased risk of cardiovascular events⁸⁵. The pathophysiology of Post-Acute Sequelae of SARS-CoV-2 Infection (PASC) is poorly understood, and instruments commonly used in clinical practice to assess organ function fail to correlate with patient-reported symptoms. Identification of biological mechanisms underpinning persistent deficits will accelerate research to better understand, predict, and manage PASC. Because an organism's cells share identical genetic information, different phenotypes are established and maintained by epigenetic mechanisms¹⁶. DNA methylation is a covalent yet dynamic epigenetic modification that influences gene expression profiles, especially when present in gene promoter regions¹⁶. Differentially methylated regions (DMRs) comprise serial cytosine-guanine dinucleotide sites (CpG) positions that are consecutively hyper- or hypo-methylated and can persist over long periods of time⁸⁶. Accordingly, DNA methylation is a plausible mechanism to maintain an abnormal cellular phenotype after resolution of acute disease. Because PASC is caused by prior SARS-CoV-2 infection and host inflammatory responses, circulating leukocytes are attractive targets to investigate differential DNA methylation induced by acute infection. We have shown that SARS-CoV-2 infection disrupts the circulating leukocyte DNA methylome⁸⁷ and transcriptome¹¹ in correlation with disease severity spanning full recovery to death. We found that SARS-CoV-2 infection is characterized by 1,505 DMRs compared to healthy control individuals, and gene ontological analysis indicates that these genes participate in immune responses, leukocyte activation, viral responses, and related processes. Thus, we reasoned that a subset of these SARS-CoV-2 DMRs could endure long after recovery from COVID-19.

### Methods

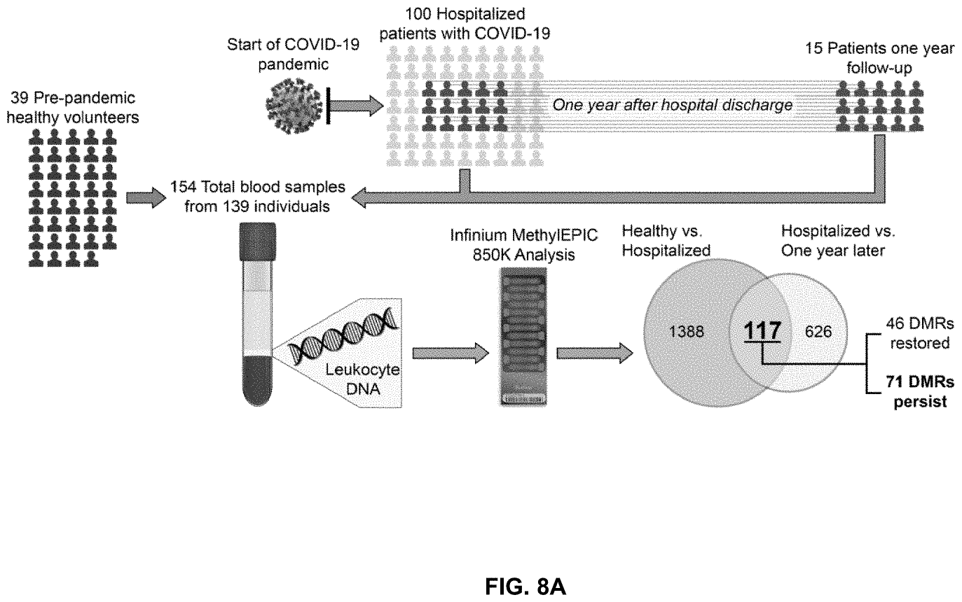

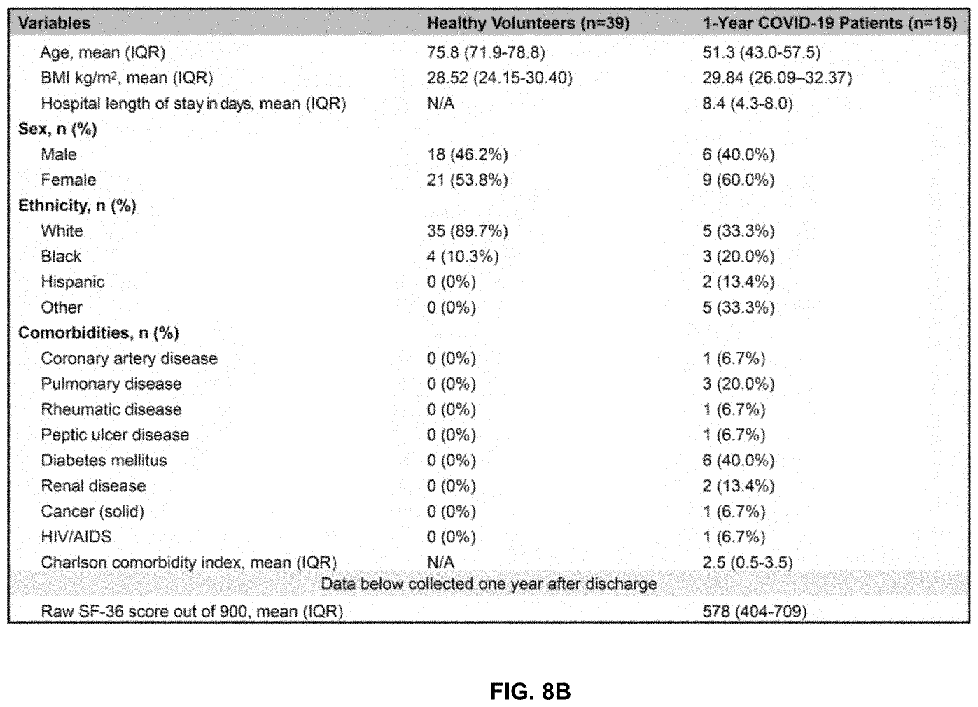

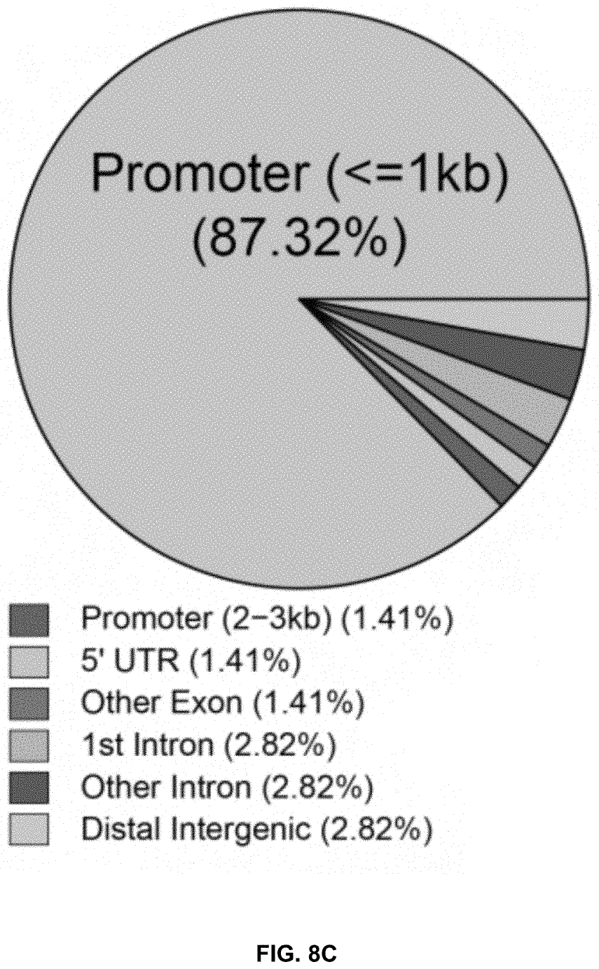

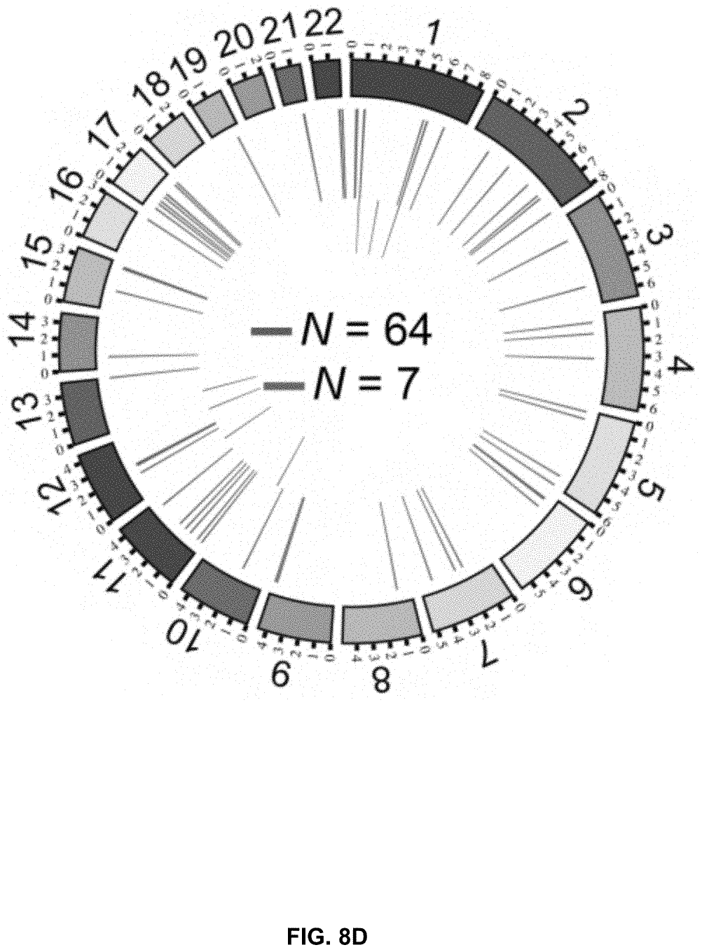

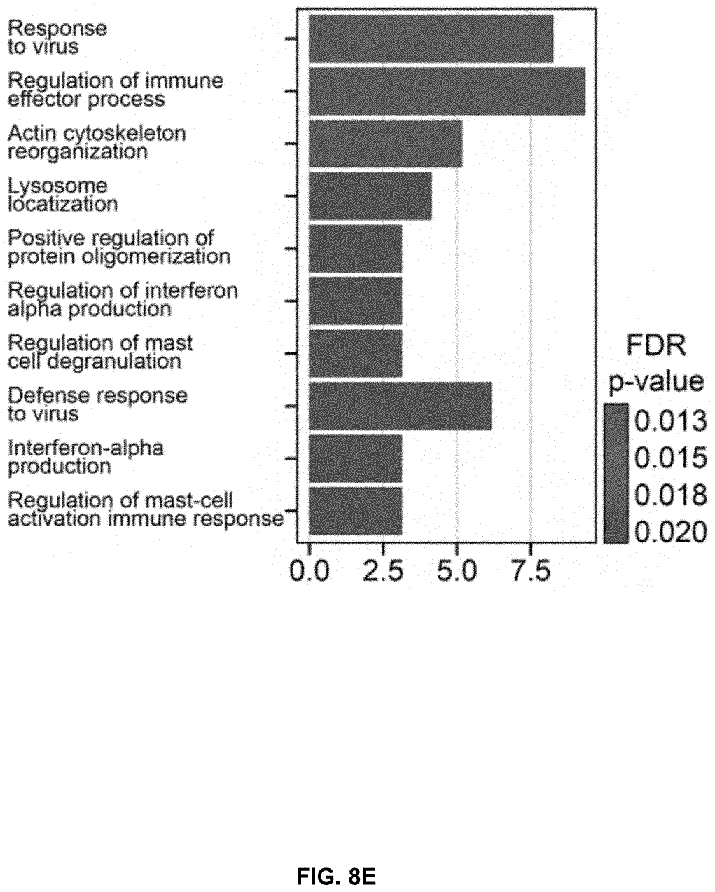

To investigate this hypothesis, all the participants from our original cohort who survived COVID-19 hospitalization between March and April 2020 were recontacted 1 year after discharge (FIGS. 8A and 8B). Specific description of the cohort can be found in previous publications^{11,87-88}. Fifteen patients out of the original 102 participants and corresponding to 30% of surviving individuals consented to a second office visit for clinical evaluation and a new blood sample for further sequencing. Upon evaluation, these patients expressed multiple PASC symptoms including fatigue, sleep disturbances, and reduced general heath scores. However, they denied dyspnea and showed normal hemoglobin oxygen saturation while breathing ambient air. As DNA methylation status is not yet annotated, we used samples from 39 healthy volunteers which were analyzed with the same platform as previously reported⁸⁷. These participants were enrolled before the current pandemic, ruling out possible differential DNA methylation caused by asymptomatic COVID-19 infection. Healthy participants were older than SARS-CoV-2 (78 versus 51 years old, respectively); other characteristics are presented in FIG. 8B. Leukocyte DNA was purified and bisulfite-converted for DNA methylation analysis using the Infinium Human MethylationEPIC 850K BeadChip on an Illumina® platform. To adjust for batch effects, and given that these were patient-matched specimens, the model used for differential methylation was adjusted for patient ID. Following model selection, R packages ComBat and SVA were employed to adjust for known batch effects and latent confounding variables, respectively, and were adjusted for in the model. Differential methylation analysis was then performed as recently reported⁸⁷.

### Results

Of the 1,505 acute illness induced DMRs we previously identified⁸⁷, 71 DMRs remained significantly differentially methylated 1 year thereafter, with an average of 7 serial CpG positions per DMR. Sixty-four DMRs remained hypermethylated, and 7 DMR remained hypomethylated (p<0.0001). Over 90% of the lasting DMRs were located near or within gene promoter regions (FIG. 8C), suggesting an effect on gene expression regulation¹⁶. DMRs were uniformly distributed along the entire genome (FIG. 8D). Because of X chromosome inactivation in females, vast differences in DNA methylation abundances may be observed between females and males. Additionally, normalization of DNA methylation data via microarray technology may introduce a technical bias to CpGs harbored on autosomes. Moreover, removal of sex chromosome related CpGs that may introduce a technical bias reduces the number of multiple comparisons to correct for during differential analysis. For these reasons, sex chromosomes were omitted from the analysis. Gene ontological (GO) enrichment analysis of the genes harboring the lasting DMRs included pathways related to viral responses and inflammation (FIG. 8E), see also accession numbers GSE174818 and GSE197152. For details regarding the specific genes that remain dysregulated one year after hospital discharge, and their corresponding chromosomal location, we provide the readers with that information in the Table 21.

### Discussion

More than 6 million deaths have been attributed to COVID-19, primarily arising from acute respiratory failure¹. Recent data indicate that disease severity predominantly depends on host factors^{5,6}, supporting the need to better differentiate individual responses at the molecular level. We and others have described outcome-specific multi-omic profiles of COVID-19 patients^{11,13,87}. However specific host mechanisms that coordinate expression of these profiles are unresolved. While an individual's nucleated cells share identical genomic sequences, distinct cellular phenotypes are established and maintained by epigenetic mechanisms^{14,15}, including DNA methylation, histone and chromatin modifications, and non-coding RNA transcription¹⁶. DNA methylation regulates gene expression and is sensitive to environmental factors^{16,19-23}. As previously described, methylation of CpGs located at promoter regions is canonically associated with transcriptional repression¹⁶. Mechanistically, methylated CpGs recruit complexes holding methyl-CpG binding domain-containing proteins and other factors that aggregate into multiprotein repressive complexes to silence transcription^{89,90}. Critically ill patients have altered circulating blood DNA methylation profiles^{24,25}, consistent with epigenetic regulation of gene expression. We have recently reported a genome-wide DNA methylation analysis of patients with COVID-19 in correlation with clinical outcomes spanning full recovery to death and multiple sources have reported that DNA methylation is relevant in the pathophysiology of acute COVID-19 infection⁹¹⁻⁹³. These findings introduce evidence of acute epigenetic regulation of genes associated with COVID-19 severity⁸⁷. Although many patients who survive COVID-19 develop long term cognitive and somatic dysfunctions⁹⁴, no pathobiological processes that account for these lingering deficits have been identified. We present here evidence that epigenetic marks can persist beyond clinical resolution of acute illness. These data are the first reported evidence that DNA methylation changes in circulating leukocytes endure at least 1 year after recovery from acute COVID-19 illness, leaving durable marks in the methylome that we predict condition patterns of gene expression that regulate PASC pathophysiology. Accordingly, DNA methylation may be a mechanism regulating leukocyte adhesion and vascular injury and contribute to the recently described higher risk of cardiovascular events after COVID-19⁸⁵.

### REFERENCES

1 Zhou, F. et al. Clinical course and risk factors for mortality of adult inpatients with COVID-19 in Wuhan, China: a retrospective cohort study. Lancet 395, 1054-1062, doi:10.1016/S0140-6736(20)30566-3 (2020).
2 Wu, C. et al. Risk Factors Associated With Acute Respiratory Distress Syndrome and Death in Patients With Coronavirus Disease 2019 Pneumonia in Wuhan, China. JAMA Intern Med, doi:10.1001/jamainternmed.2020.0994 (2020).
3 Wang, Y. et al. Clinical Course and Outcomes of 344 Intensive Care Patients with COVID-19. Am J Respir Crit Care Med 201, 1430-1434, doi:10.1164/rccm.202003-0736LE (2020).
4 Lucas, C. et al. Longitudinal analyses reveal immunological misfiring in severe COVID-19. Nature, doi:10.1038/s41586-020-2588-y (2020).
5 Zhang, X. et al. Viral and host factors related to the clinical outcome of COVID-19. Nature 583, 437-440, doi:10.1038/s41586-020-2355-0 (2020).
6 Dorward, D. A. et al. Tissue-specific Immunopathology in Fatal COVID-19. Am J Respir Crit Care Med, doi:10.1164/rccm.202008-3265OC (2020).
7 Group, R. C. et al. Dexamethasone in Hospitalized Patients with Covid-19-Preliminary Report. N Engl J Med, doi:10.1056/NEJMoa2021436 (2020).
8 Baillie, J. K. Translational genomics. Targeting the host immune response to fight infection. Science 344, 807-808, doi:10.1126/science.1255074 (2014).
9 Pairo-Castineira, E. et al. Genetic mechanisms of critical illness in Covid-19. Nature, doi:10.1038/s41586-020-03065-y (2020).
10 Bernardes, J. P. et al. Longitudinal Multi-omics Analyses Identify Responses of Megakaryocytes, Erythroid Cells, and Plasmablasts as Hallmarks of Severe COVID-19. Immunity 53, 1296-1314 e1299, doi:10.1016/j.immuni.2020.11.017 (2020).
11 Overmyer, K. A. et al. Large-Scale Multi-omic Analysis of COVID-19 Severity. Cell Syst, doi:10.1016/j.cels.2020.10.003 (2020).
12 Shen, B. et al. Proteomic and Metabolomic Characterization of COVID-19 Patient Sera. Cell, doi:10.1016/j.cell.2020.05.032 (2020).
13 Arunachalam, P. S. et al. Systems biological assessment of immunity to mild versus severe COVID-19 infection in humans. Science 369, 1210-1220, doi:10.1126/science.abc6261 (2020).
14 Waddington, C. H. The epigenotype. 1942. Int J Epidemiol 41, 10-13, doi:10.1093/ije/dyr184 (2012).
15 Goll, M. G. & Bestor, T. H. Eukaryotic cytosine methyltransferases. Annu Rev Biochem 74, 481-514, doi:10.1146/annurev.biochem.74.010904.153721 (2005).
16 Singer, B. D. A Practical Guide to the Measurement and Analysis of DNA Methylation. Am J Respir Cell Mol Biol 61, 417-428, doi:10.1165/rcmb.2019-0150TR (2019).
17 Berger, S. L., Kouzarides, T., Shiekhattar, R. & Shilatifard, A. An operational definition of epigenetics. Genes Dev 23, 781-783, doi:10.1101/gad.1787609 (2009).
18 Goldberg, A. D., Allis, C. D. & Bernstein, E. Epigenetics: a landscape takes shape. Cell 128, 635-638, doi:10.1016/j.cell.2007.02.006 (2007).
19 Helmin, K. A. et al. Maintenance DNA methylation is essential for regulatory T cell development and stability of suppressive function. J Clin Invest 130, 6571-6587, doi:10.1172/JCI137712 (2020).
20 Walter, J. M., Helmin, K. A., Abdala-Valencia, H., Wunderink, R. G. & Singer, B. D. Multidimensional assessment of alveolar T cells in critically ill patients. JCI Insight 3, doi:10.1172/jci.insight.123287 (2018).
21 Madrid, A. et al. DNA Hypomethylation in Blood Links B3GALT4 and ZADH2 to Alzheimer's Disease. J Alzheimers Dis 66, 927-934, doi:10.3233/JAD-180592 (2018).
22 Alisch, R. S. et al. Age-associated DNA methylation in pediatric populations. Genome Res 22, 623-632, doi:10.1101/gr.125187.111 (2012).
23 Patel, N. J. et al. Ancestral Folate Promotes Neuronal Regeneration in Serial Generations of Progeny. Mol Neurobiol 57, 2048-2071, doi:10.1007/s12035-019-01812-5 (2020).
24 Binnie, A. et al. Epigenetic Profiling in Severe Sepsis: A Pilot Study of DNA Methylation Profiles in Critical Illness. Crit Care Med, doi:10.1097/CCM.0000000000004097 (2019).
25 Guiza, F. et al. Effect of early parenteral nutrition during paediatric critical illness on DNA methylation as a potential mediator of impaired neurocognitive development: a pre-planned secondary analysis of the PEPaNIC international randomised controlled trial. Lancet Respir Med 8, 288-303, doi:10.1016/S2213-2600(20)30046-1 (2020).
26 Schafer, A. & Baric, R. S. Epigenetic Landscape during Coronavirus Infection. Pathogens 6, doi:10.3390/pathogens6010008 (2017).
27 Fang, T. C. et al. Histone H3 lysine 9 di-methylation as an epigenetic signature of the interferon response. J Exp Med 209, 661-669, doi: 10.1084/jem.20112343 (2012).
28 Menachery, V. D. et al. Pathogenic influenza viruses and coronaviruses utilize similar and contrasting approaches to control interferon-stimulated gene responses. mBio 5, e01174-01114, doi:10.1128/mBio.01174-14 (2014).
29 Menachery, V. D. et al. MERS-CoV and H5N1 influenza virus antagonize antigen presentation by altering the epigenetic landscape. Proc Natl Acad Sci USA 115, E1012-E1021, doi:10.1073/pnas.1706928115 (2018).
30 Davenport, E. E. et al. Genomic landscape of the individual host response and outcomes in sepsis: a prospective cohort study. Lancet Respir Med 4, 259-271, doi:10.1016/52213-2600(16)00046-1 (2016).
31 Bos, L. D. J. et al. Understanding Heterogeneity in Biologic Phenotypes of Acute Respiratory Distress Syndrome by Leukocyte Expression Profiles. Am J Respir Crit Care Med 200, 42-50, doi:10.1164/rccm.201809-1808OC (2019).
32 Crimi, E., Benincasa, G., Figueroa-Marrero, N., Galdiero, M. & Napoli, C. Epigenetic susceptibility to severe respiratory viral infections and its therapeutic implications: a narrative review. Br J Anaesth 125, 1002-1017, doi:10.1016/j.bja.2020.06.060 (2020).
33 Atlante, S. et al. The epigenetic implication in coronavirus infection and therapy. Clin Epigenetics 12, 156, doi:10.1186/s13148-020-00946-x (2020).
34 Calfee, C. S. et al. Subphenotypes in acute respiratory distress syndrome: latent class analysis of data from two randomised controlled trials. Lancet Respir Med 2, 611-620, doi:10.1016/52213-2600(14)70097-9 (2014).
35 Calfee, C. S. et al. Acute respiratory distress syndrome subphenotypes and differential response to simvastatin: secondary analysis of a randomised controlled trial. Lancet Respir Med 6, 691-698, doi:10.1016/52213-2600(18)30177-2 (2018).
36 Bates, S. E. Epigenetic Therapies for Cancer. N Engl J Med 383, 650-663, doi:10.1056/NEJMra1805035 (2020).
37 Tomazini, B. M. et al. Effect of Dexamethasone on Days Alive and Ventilator-Free in Patients With Moderate or Severe Acute Respiratory Distress Syndrome and COVID-19: The CoDEX Randomized Clinical Trial. JAMA 324, 1307-1316, doi:10.1001/jama.2020.17021 (2020).
38 Angus, D. C. et al. Effect of Hydrocortisone on Mortality and Organ Support in Patients With Severe COVID-19: The REMAP-CAP COVID-19 Corticosteroid Domain Randomized Clinical Trial. JAMA 324, 1317-1329, doi:10.1001/jama.2020.17022 (2020).
39 Prescott, H. C. & Rice, T. W. Corticosteroids in COVID-19 ARDS: Evidence and Hope During the Pandemic. JAMA, doi:10.1001/jama.2020.16747 (2020).
40 Liang, W. et al. Development and Validation of a Clinical Risk Score to Predict the Occurrence of Critical Illness in Hospitalized Patients With COVID-19. JAMA Intern Med 180, 1081-1089, doi:10.1001/jamainternmed.2020.2033 (2020).
41 Webb Hooper, M., Napoles, A. M. & Perez-Stable, E. J. COVID-19 and Racial/Ethnic Disparities. JAMA 323, 2466-2467, doi:10.1001/jama.2020.8598 (2020).
42 Ferreira, F. L., Bota, D. P., Bross, A., Melot, C. & Vincent, J. L. Serial evaluation of the SOFA score to predict outcome in critically ill patients. JAMA 286, 1754-1758 (2001).
43 Charlson, M. E., Pompei, P., Ales, K. L. & MacKenzie, C. R. A new method of classifying prognostic comorbidity in longitudinal studies: development and validation. J Chronic Dis 40, 373-383, doi:10.1016/0021-9681(87)90171-8 (1987).
44 Peters, T. J. et al. De novo identification of differentially methylated regions in the human genome. Epigenetics Chromatin 8, 6, doi:10.1186/1756-8935-8-6 (2015).
45 Bell, C. G. et al. DNA methylation aging clocks: challenges and recommendations. Genome Biol 20, 249, doi:10.1186/s13059-019-1824-y (2019).
46 Gindin, Y. et al. DNA Methylation and Immune Cell Markers Demonstrate Evidence of Accelerated Aging in Patients with Chronic HBV or HCV, with or without HIV Co-Infection. Clin Infect Dis, doi:10.1093/cid/ciaal371 (2020).
47 M, K. Building predictive models in r using the caret package. J Stat Software 28, 1-26 (2008).
48 Binnie, A. et al. Epigenetic Profiling in Severe Sepsis: A Pilot Study of DNA Methylation Profiles in Critical Illness. Crit Care Med 48, 142-150, doi:10.1097/CCM.0000000000004097 (2020).
49 Paschos, K. & Allday, M. J. Epigenetic reprogramming of host genes in viral and microbial pathogenesis. Trends Microbiol 18, 439-447, doi:10.1016/j.tim.2010.07.003 (2010).
50 Verlinden, I. et al. Time course of altered DNA methylation evoked by critical illness and by early administration of parenteral nutrition in the paediatric ICU. Clin Epigenetics 12, 155, doi:10.1186/s13148-020-00947-w (2020).
51 Imgenberg-Kreuz, J. et al. Genome-wide DNA methylation analysis in multiple tissues in primary Sjogren's syndrome reveals regulatory effects at interferon-induced genes. Ann Rheum Dis 75, 2029-2036, doi:10.1136/annrheumdis-2015-208659 (2016).
52 Coit, P. et al. Genome-wide DNA methylation study suggests epigenetic accessibility and transcriptional poising of interferon-regulated genes in naive CD4+ T cells from lupus patients. J Autoimmun 43, 78-84, doi:10.1016/j.jaut.2013.04.003 (2013).
53 Coit, P. et al. Epigenome profiling reveals significant DNA demethylation of interferon signature genes in lupus neutrophils. J Autoimmun 58, 59-66, doi:10.1016/j.jaut.2015.01.004 (2015).
54 Chen, S. et al. Genome-Wide DNA Methylation Profiles Reveal Common Epigenetic Patterns of Interferon-Related Genes in Multiple Autoimmune Diseases. Front Genet 10, 223, doi:10.3389/fgene.2019.00223 (2019).
55 Absher, D. M. et al. Genome-wide DNA methylation analysis of systemic lupus erythematosus reveals persistent hypomethylation of interferon genes and compositional changes to CD4+ T-cell populations. PLoS Genet 9, e1003678, doi:10.1371/journal.pgen.1003678 (2013).
56 Ehrenfeld, M. et al. Covid-19 and autoimmunity. Autoimmun Rev 19, 102597, doi:10.1016/j.autrev.2020.102597 (2020).
57 Canas, C. A. The triggering of post-COVID-19 autoimmunity phenomena could be associated with both transient immunosuppression and an inappropriate form of immune reconstitution in susceptible individuals. Med Hypotheses 145, 110345, doi:10.1016/j.mehy.2020.110345 (2020).
58 Oriol-Tordera, B. et al. Methylation regulation of Antiviral host factors, Interferon Stimulated Genes (ISGs) and T-cell responses associated with natural HIV control. PLoS Pathog 16, e1008678, doi:10.1371/journal.ppat.1008678 (2020).
59 Pontelli M C, C. I., Martins R B, Veras F P, La Serra L, Nascimento D C, Cardoso R S, Rosales R, Lima™, Souza J P, Caetite D B, De Lima M H F, Kawahisa J T, Giannini M C, Bonjorno L P, Lopes M I F, Batah S S, Siyuan L, Assad R L, Almeida S C L, Olivera F R, Benatti M N, Pontes L L F, Santana R C, Villar F C, Martins M A, Cunha™, Calado R T, Alves-Filho Marjorie C Pontelli, Italo A Castro, Ronaldo B Martins, Flávio P Veras, Leonardo La Serra, Daniele C Nascimento, Ricardo S Cardoso, Roberta Rosales, Thais M Lima, Juliano P Souza, Diego B Caetité, Mikhael H F de Lima, Juliana T Kawahisa, Marcela C Giannini, Leticia P Bonjorno, Maria I F Lopes, Sabrina S Batah, Li Siyuan, Rodrigo L Assad, Sergio C L Almeida, Fabiola R Oliveira, Maira N Benatti, Lorena L F Pontes, Rodrigo C Santana, Fernando C Vilar, Maria A Martins, Thiago M Cunha, Rodrigo T Calado, Jose C Alves-Filho, Dario S Zamboni, Alexandre Fabro, Paulo Louzada-Junior, Rene D R Oliveira, Fernando Q Cunha, Eurico Arruda. Infection of human lymphomononuclear cells by SARS-CoV-2. BioRxiv, doi.org/10.1101/2020.07.28.225912 (2020).
60 Tournier-Lasserve, E., Odenwald, W. F., Garbern, J., Trojanowski, J. & Lazzarini, R. A. Remarkable intron and exon sequence conservation in human and mouse homeobox Hox 1.3 genes. Mol Cell Biol 9, 2273-2278, doi:10.1128/mcb.9.5.2273 (1989).
61 Pachot, A. et al. Messenger RNA expression of major histocompatibility complex class II genes in whole blood from septic shock patients. Crit Care Med 33, 31-38; discussion 236-237, doi:10.1097/01.ccm.0000150958.20209.a3 (2005).
62 Boomer, J. S. et al. Immunosuppression in patients who die of sepsis and multiple organ failure. JAMA 306, 2594-2605, doi:10.1001/jama.2011.1829 (2011).
63 Avendano-Ortiz, J. et al. Proteins from SARS-CoV-2 reduce T cell proliferation: A mirror image of sepsis. Heliyon 6, e05635, doi:10.1016/j.heliyon.2020.e05635 (2020).
64 Sinha, P., Churpek, M. M. & Calfee, C. S. Machine Learning Classifier Models Can Identify Acute Respiratory Distress Syndrome Phenotypes Using Readily Available Clinical Data. Am J Respir Crit Care Med 202, 996-1004, doi:10.1164/rccm.202002-0347OC (2020).
65 Sinha, P., Matthay, M. A. & Calfee, C. S. Is a “Cytokine Storm” Relevant to COVID-19? JAMA Intern Med, doi:10.1001/jamainternmed.2020.3313 (2020).
66 Bos, L. D. et al. Identification and validation of distinct biological phenotypes in patients with acute respiratory distress syndrome by cluster analysis. Thorax 72, 876-883, doi:10.1136/thoraxjnl-2016-209719 (2017).
67 Corley, M. J. et al. Genome-wide DNA methylation profiling of peripheral blood reveals an epigenetic signature associated with severe COVID-19. J Leukoc Biol, doi:10.1002/JLB.5HI0720-466R (2021).
68 Antcliffe, D. B. et al. Transcriptomic Signatures in Sepsis and a Differential Response to Steroids. From the VANISH Randomized Trial. Am J Respir Crit Care Med 199, 980-986, doi:10.1164/rccm.201807-1419OC (2019).
69 Herridge, M. S. et al. One-year outcomes in survivors of the acute respiratory distress syndrome. N Engl J Med 348, 683-693, doi:10.1056/NEJMoa022450 (2003).
70 Herridge, M. S. et al. The RECOVER Program: Disability Risk Groups and 1-Year Outcome after 7 or More Days of Mechanical Ventilation. Am J Respir Crit Care Med 194, 831-844, doi:10.1164/rccm.201512-2343OC (2016).
71 Iwashyna, T. J., Ely, E. W., Smith, D. M. & Langa, K. M. Long-term cognitive impairment and functional disability among survivors of severe sepsis. JAMA 304, 1787-1794, doi:10.1001/jama.2010.1553 (2010).
72 Pandharipande, P. P. et al. Long-term cognitive impairment after critical illness. N Engl J Med 369, 1306-1316, doi:10.1056/NEJMoa1301372 (2013).
73 Helms, J. et al. Neurologic Features in Severe SARS-CoV-2 Infection. N Engl J Med 382, 2268-2270, doi:10.1056/NEJMc2008597 (2020).
74 Arnold, C. Could COVID delirium bring on dementia? Nature 588, 22-24, doi:10.1038/d41586-020-03360-8 (2020).
75 Sakr, Y. et al. The Intensive Care Global Study on Severe Acute Respiratory Infection (IC-GLOSSARI): a multicenter, multinational, 14-day inception cohort study. Intensive Care Med 42, 817-828, doi:10.1007/s00134-015-4206-2 (2016).
76 Fransquet, P. D. et al. Blood DNA methylation as a potential biomarker of dementia: A systematic review. Alzheimers Dement 14, 81-103, doi:10.1016/j.jalz.2017.10.002 (2018).
77 El Baba, R. & Herbein, G. Management of epigenomic networks entailed in coronavirus infections and COVID-19. Clin Epigenetics 12, 118, doi:10.1186/s13148-020-00912-7 (2020).
78 Wang, D. et al. Clinical Characteristics of 138 Hospitalized Patients With 2019 Novel Coronavirus-Infected Pneumonia in Wuhan, China. JAMA, doi:10.1001/jama.2020.1585 (2020).
79 Moran, S., Arribas, C. & Esteller, M. Validation of a DNA methylation microarray for 850,000 CpG sites of the human genome enriched in enhancer sequences. Epigenomics 8, 389-399, doi:10.2217/epi.15.114 (2016).
80 Aryee, M. J. et al. Minfi: a flexible and comprehensive Bioconductor package for the analysis of Infinium DNA methylation microarrays. Bioinformatics 30, 1363-1369, doi:10.1093/bioinformatics/btu049 (2014).
81 Fortin, J. P. et al. Functional normalization of 450k methylation array data improves replication in large cancer studies. Genome Biol 15, 503, doi:10.1186/s13059-014-0503-2 (2014).
82 Leek, J. T., Johnson, W. E., Parker, H. S., Jaffe, A. E. & Storey, J. D. The sva package for removing batch effects and other unwanted variation in high-throughput experiments. Bioinformatics 28, 882-883, doi:10.1093/bioinformatics/bts034 (2012).
83 Yu, G., Wang, L. G., Han, Y. & He, Q. Y. clusterProfiler: an R package for comparing biological themes among gene clusters. OMICS 16, 284-287, doi:10.1089/omi.2011.0118 (2012).
84 Cui, Y. et al. BioCircos.js: an interactive Circos JavaScript library for biological data visualization on web applications. Bioinformatics 32, 1740-1742, doi:10.1093/bioinformatics/btw041 (2016).
85. Xie, Y., Xu, E., Bowe, B. & Al-Aly, Z. Long-term cardiovascular outcomes of COVID-19. Nat Med, doi:10.1038/s41591-022-01689-3 (2022).
86. Sharif, J. et al. The SRA protein Np95 mediates epigenetic inheritance by recruiting Dnmt1 to methylated DNA. Nature 450, 908-912, doi:10.1038/nature06397 (2007).
87. Balnis, J. et al. Blood DNA methylation and COVID-19 outcomes. Clin Epigenetics 13, 118, doi:10.1186/s13148-021-01102-9 (2021).
88. Balnis, J. et al. Unique inflammatory profile is associated with higher SARS-CoV-2 acute respiratory distress syndrome (ARDS) mortality. Am J Physiol Regul Integr Comp Physiol 320, R250-R257, doi:10.1152/ajpregu.00324.2020 (2021).
89. Amabile, A. et al. Inheritable Silencing of Endogenous Genes by Hit-and-Run Targeted Epigenetic Editing. Cell 167, 219-232 e214, doi:10.1016/j.cell.2016.09.006 (2016).
90. Lyko, F. The DNA methyltransferase family: a versatile toolkit for epigenetic regulation. Nat Rev Genet 19, 81-92, doi:10.1038/nrg.2017.80 (2018).
91. Morselli, E. et al. Spermidine and resveratrol induce autophagy by distinct pathways converging on the acetylproteome. J Cell Biol 192, 615-629, doi:10.1083/jcb.201008167 (2011).
92. Castro de Moura, M. et al. Epigenome-wide association study of COVID-19 severity with respiratory failure. EBioMedicine, 103339, doi:10.1016/j.ebiom.2021.103339 (2021).
93. Saiz, M. L. et al. Epigenetic targeting of the ACE2 and NRP1 viral receptors limits SARS-CoV-2 infectivity. Clin Epigenetics 13, 187, doi:10.1186/s13148-021-01168-5 (2021).
94. Al-Aly, Z., Xie, Y. & Bowe, B. High-dimensional characterization of post-acute sequalae of COVID-19. Nature, doi:10.1038/s41586-021-03553-9 (2021).
95. Franzen, J. et al. Epigenetic Clocks Are Not Accelerated in COVID-19 Patients. Int J Mol Sci 22, doi:10.3390/ijms22179306 (2021).

1. A method comprising:
determining a methylation level of any one or more CpG sites in any one or more genomic regions of a genome of a subject, wherein the one or more genomic regions comprise one or more of:
positions 40024971-40025415 of chromosome 1;
positions 63249197-63249213 of chromosome 1;
positions 95698827-95699097 of chromosome 1;
positions 154127462-154128443 of chromosome 1;
positions 162467080-162467363 of chromosome 1;
positions 27301195-27301943 of chromosome 2;
positions 47382287-47382903 of chromosome 2;
positions 121223534-121223964 of chromosome 2;
positions 42756397-42757171 of chromosome 5;
positions 102898463-102898733 of chromosome 5;
positions 110062384-110062618 of chromosome 5;
positions 138210550-138211184 of chromosome 5;
positions 180257691-180257804 of chromosome 5;
positions 28058187-28059208 of chromosome 6;
positions 28829283-28829674 of chromosome 6;
positions 32164503-32165200 of chromosome 6;
positions 168393930-168394160 of chromosome 6;
positions 965379-965534 of chromosome 7;
positions 151433178-151433561 of chromosome 7;
positions 16859295-16860121 of chromosome 8;
positions 144635260-144635610 of chromosome 8;
positions 76803669-76803925 of chromosome 10;
positions 2334892-2335053 of chromosome 11;
positions 2891077-2891118 of chromosome 11;
positions 15037755-15039432 of chromosome 12;
positions 49107116-49108131 of chromosome 13;
positions 110438578-110439234 of chromosome 13;
positions 112861499-112861518 of chromosome 13;
positions 72053146-72053361 of chromosome 14;
positions 104394430-104394831 of chromosome 14;
positions 101389272-101389394 of chromosome 15;
positions 3355951-3356149 of chromosome 16;
positions 49563759-49564462 of chromosome 16;
positions 67034309-67034882 of chromosome 16;
positions 75681737-75682004 of chromosome 16;
positions 75568999-75569749 of chromosome 16;
positions 6797466-6797771 of chromosome 17;
positions 8066669-8067323 of chromosome 17;
positions 11784246-11785188 of chromosome 19;
positions 52390810-52391789 of chromosome 19;
positions 5485144-5486007 of chromosome 20; and
positions 61583686-61584248 of chromosome 20.

2. The method of claim 1, wherein the determining comprises determining the methylation level of each CpG site in any one or more of the genomic regions.

3. The method of claim 1, wherein the determining comprises determining the methylation level of each CpG site in any five or more of the genomic regions.

4. The method of claim 1, wherein the determining comprises determining the methylation level of any one or more CpG sites in any five or more of the genomic regions.

5. The method of claim 1, wherein the determining comprises determining the methylation level of any one or more CpG sites in each of the genomic regions.

6. The method of claim 1, the determining comprises determining the methylation level of each CpG site in each of the genomic regions.

7. The method of claim 1, wherein a methylation level is determined for no more than 800,000 CpG sites in the genome of the subject.

8. The method of claim 1, wherein the subject is a subject presenting with respiratory illness.

9. The method of claim 1, wherein the subject is a subject infected or suspected of being infected with coronavirus.

10. The method of claim 1, wherein the subject is a subject infected or suspected of being infected with SARS-CoV-2.

11. The method of claim 1, wherein the subject is a subject diagnosed with SARS-CoV-2 infection.

12. The method of claim 1, wherein the determining comprises:
treating genomic DNA from the subject with bisulfite to generate bisulfite-treated genomic DNA;
amplifying the bisulfite-treated genomic DNA using primers specific for a portion of the bisulfite-treated genomic DNA comprising the one or more genomic regions; and
measuring the methylation level of the one or more CpG sites in the one or more genomic regions.

13. The method of claim 12, wherein the portion of the bisulfite-treated genomic DNA has a length less than 1000 bases.

14. The method of claim 1, wherein the methylation level is measured by methylation-specific PCR, quantitative methylation-specific PCR, methylation-sensitive DNA restriction enzyme analysis, or bisulfite genomic sequencing PCR, or quantitative bisulfite pyrosequencing.

15. The method of claim 1, wherein the one or more genomic regions comprise one or more of:
positions 40024971-40025415 of chromosome 1;
positions 95698827-95699097 of chromosome 1;
positions 162467080-162467363 of chromosome 1;
positions 27301195-27301943 of chromosome 2;
positions 47382287-47382903 of chromosome 2;
positions 42756397-42757171 of chromosome 5;
positions 102898463-102898733 of chromosome 5;
positions 138210550-138211184 of chromosome 5;
positions 28829283-28829674 of chromosome 6;
positions 168393930-168394160 of chromosome 6;
positions 965379-965534 of chromosome 7;
positions 2334892-2335053 of chromosome 11;
positions 2891077-2891118 of chromosome 11;
positions 110438578-110439234 of chromosome 13;
positions 101389272-101389394 of chromosome 15;
positions 3355951-3356149 of chromosome 16;
positions 11784246-11785188 of chromosome 19;
positions 52390810-52391789 of chromosome 19; and
positions 61583686-61584248 of chromosome 20.

16. The method of claim 15, wherein the one or more CpG sites are located at any one or more of the genomic positions shown in Table 15.

17. The method of claim 15, further comprising determining from the methylation level of the one or more CpG sites a likelihood of developing critical illness comprising at least one of admission to an intensive care unit, invasive ventilation, and death.

18. The method of claim 1, wherein the one or more genomic regions comprise one or more of:
positions 63249197-63249213 of chromosome 1;
positions 27301195-27301943 of chromosome 2;
positions 121223534-121223964 of chromosome 2;
positions 110062384-110062618 of chromosome 5;
positions 180257691-180257804 of chromosome 5;
positions 28058187-28059208 of chromosome 6;
positions 32164503-32165200 of chromosome 6;
positions 151433178-151433561 of chromosome 7;
positions 16859295-16860121 of chromosome 8;
positions 144635260-144635610 of chromosome 8;
positions 76803669-76803925 of chromosome 10;
positions 112861499-112861518 of chromosome 13;
positions 72053146-72053361 of chromosome 14;
positions 104394430-104394831 of chromosome 14;
positions 49563759-49564462 of chromosome 16;
positions 67034309-67034882 of chromosome 16;
positions 75568999-75569749 of chromosome 16; and
positions 6797466-6797771 of chromosome 17.

19. The method of claim 18, wherein the subject is a subject infected with SARS-CoV-2 and the method further comprises determining from the methylation level of the one or more CpG sites a likelihood of dying from the SARS-CoV-2 infection.

20. The method of claim 1, wherein the one or more genomic regions comprise one or more of:
positions 154127462-154128443 of chromosome 1;
positions 15037755-15039432 of chromosome 12;
positions 49107116-49108131 of chromosome 13;
positions 75681737-75682004 of chromosome 16;
positions 8066669-8067323 of chromosome 17; and
positions 5485144-5486007 of chromosome 20.

21. The method of claim 20, wherein the one or more CpG sites are located at any one or more of the genomic positions shown in Table 17A.

22. The method of claim 20, wherein the subject is a subject infected with SARS-CoV-2, and wherein the method further comprises determining from the methylation level of the one or more CpG sites a likelihood of the subject responding to treatment.

23. The method of claim 22, further comprising treating the subject.

24. The method of claim 23, wherein the treating comprising treating with a steroid.
